(19) United States
(12) Patent Application Publication
(10) Pub. No.: US 2011/0195068 A1
(43) Pub. Date: Aug. 11, 2011

(54) PD-1 ANTAGONISTS AND METHODS OF USE THEREOF

(76) Inventors: Solomon Langermann, Baltimore, MD (US); Linda Liu, Clarksville, MD (US)

(21) Appl. No.: 13/060,998

(22) PCT Filed: Aug. 25, 2009
(86) PCT No.: PCT/US09/54971

§ 371 Date: Feb. 25, 2011

Related U.S. Application Data
(60) Provisional application No. 61/091,705, filed on Aug. 25, 2008.
(60) Provisional application No. 61/091,694, filed on Aug. 25, 2008.
(60) Provisional application No. 61/091,709, filed on Aug. 25, 2008.
(60) Provisional application No. 61/091,502, filed on Aug. 25, 2008.
(60) Provisional application No. 61/142,548, filed on Jan. 5, 2009.
(60) Provisional application No. 61/165,652, filed on Apr. 1, 2009.

(51) Int. Cl. A61K 39/395; A61K 38/17; C07K 14/16; C07K 16/18; A61P 35/00; A61P 37/04

(52) U.S. Cl. 424/133.1; 514/19.3; 530/350; 530/387.3; 514/19.5; 514/19.4

(57) ABSTRACT

Compositions and methods for enhancing and/or prolonging the activation of T cells (i.e., increasing antigen-specific proliferation of T cells, enhancing cytokine production by T cells, stimulating differentiation ad effector functions of T cells and/or promoting T cell survival) or overcoming T cell exhaustion and/or anergy are provided. Suitable compositions include PD-1 receptor antagonists that bind to and block the endogenous PD-1 receptor without triggering inhibitory signals from PD-1, or bind to and block PD-1 receptor ligands and preventing them from interacting with PD-1 receptors. Methods for using the PD-1 receptor antagonists to enhance immune responses in subjects in need thereof are provided.

### CROSS-REFERENCE TO RELATED APPLICATIONS

This application claims priority to and benefit of U.S. Provisional Application Nos. 61/091,502, 61/091,694, 61/091,709 and 61/091,705, all filed on Aug. 25, 2008, U.S. Provisional Application No. 61/142,548, filed on Jan. 5, 2009, and U.S. Provisional Application No. 61/165,652, filed on Apr. 1, 2009, and where permissible are incorporated by reference in their entireties.

### FIELD OF THE INVENTION

This invention relates to compositions and methods for modulating T-cell activation, in particular to compositions and methods for enhancing T-cell activation.

### BACKGROUND OF THE INVENTION

An antigen specific T cell response is mediated by two signals: 1) engagement of the T cell Receptor (TCR) with antigenic peptide presented in the context of MHC (signal 1), and 2) a second antigen-independent signal delivered by contact between different receptor/ligand pairs (signal 2). This “second signal” is critical in determining the type of T cell response (activation vs inhibition) as well as the strength and duration of that response, and is regulated by both positive and negative signals from costimulatory molecules, such as the B7 family of proteins. The most extensively characterized T cell costimulatory pathway is B7-CD28, in which B7-1 (CD80) and B7-2 (CD86) each can engage the activating CD28 receptor and the inhibitory CTLA-4 (CD152) receptor. In conjunction with signaling through the T cell receptor, CD28 ligation increases antigen-specific proliferation of T cells, enhances production of cytokines, activates differentiation and effector function, and promotes survival of T cells (Lenshow, et al., Annu. Rev. Immunol., 14:233-258 (1996); Chambers and Allison, Curr. Opin. Immunol., 9:396-404 (1997); and Rathmell and Thompson, Annu. Rev. Immunol., 17:781-828 (1999)). In contrast, signaling through CTLA-4 is thought to deliver a negative signal that inhibits T cell proliferation, IL-2 production, and cell cycle progression (Krummel and Allison, J. Exp. Med., 183:2533-2540 (1996); and Walunas, et al., J. Exp. Med., 183:2541-2550 (1996)). Other members of the B7 family include PD-L1 (Dong, et al., Nature Med., 5:1365-1369 (1999); and Freeman, et al., J. Exp. Med., 192:1-9 (2000)), PD-L2 (Tseng, et al., J. Exp. Med., 193:839-846 (2001); and Latchman, et al., Nature Immunol., 2:261-268 (2001)), B7-H2 (Wang, et al., Blood, 96:2808-2813 (2000); Swallow, et al., Immunity, 11:423-432 (1999); and Yoshinaga, et al., Nature, 402:827-832 (1999)), B7-H3 (Chapoval, et al., Nature Immunol., 2:269-274 (2001)) and B7-H4 (Choi, et al., J. Immunol., 171:4650-4654 (2003); Sica, et al., Immunity, 18:849-861 (2003); Prasad, et al., Immunity, 18:863-873 (2003); and Zang, et al., Proc. Natl. Acad. Sci. U.S.A., 100:10388-10392 (2003)). B7-H5 is a relatively newly discovered member of the B7 family. B7-H15 is described in PCT Publication No. WO 2006/012232. Functional studies indicate that B7-H5 is a positive regulator of T cell activity that functions to activate T cells.

PD-L1 and PD-L2 are ligands for PD-1 (programmed cell death-1), B7-H2 is a ligand for ICOS, and B7-H3, B7-H4 and B7-H5 remain orphan ligands at this time (Dong, et al., Immunol. Res., 28:39-48 (2003)).

Most B7 family molecules are expressed on the cell surface with a membrane proximal constant IgC domain and a membrane distal IgV domain. Receptors for these ligands share a common extracellular IgV-like domain. Interactions of receptor-ligand pairs are mediated predominantly through residues in the IgV domains of the ligands and receptors (Schwartz, et al., Nature Immunol., 3:427-434 (2002)). In general, IgV domains are described as having two sheets that each contain a layer of β-strands (Williams and Barclay, Annu. Rev. Immunol., 6:381-405 (1988)). The front and back sheets of CTLA-4 contain strands A′GFC′C and ABEDC,″ respectively (Ostrov, et al., Science, 290:816-819 (2000)), whereas the front and back sheets of the B7 IgV domains are composed of strands AGFCC′C″ and BED, respectively (Schwartz, et al., Nature, 410:604-608 (2001); Stamper, et al., Nature, 410:608-611 (2001); and Ikemizu, et al., Immunity, 12:51-60 (2000)). Crystallographic analysis revealed that the CTLA-4/B7 binding interface is dominated by the interaction of the CDR3-analogous loop from CTLA-4, composed of a MYPPPY motif, with a surface on B7 formed predominately by the G, F, C, C′ and C″ strands (Schwartz, et al., Nature, 410:604-608 (2001); and Stamper, et al., Nature, 410:608-611 (2001)). Data from ammo acid homologies, mutation, and computer modeling provide support for the concept that this motif also is a major B7-binding site for CD28 (Bajorath, et al., J. Mol. Graph. Model., 15:135-139 (1997)). Although the MYPPPY motif is not conserved in ICOS, studies have indicated that a related motif having the sequence FDPPPF and located at the analogous position is a major determinant for binding of ICOS to B7-H2 (Wand, et al., J. Exp. Med., 195:1033-1041 (2002)).

PD-L2 (also called B7-DC) is a relatively new member of the B7 family, and has an amino acid sequence that is about 34% identical to PD-L1 (also called B7-H1). Human and mouse PD-L2 orthologues share about 70% amino acid identity. While PD-L1 and PD-L2 transcripts are found in various tissues (Dong, et al., Nature Med., 5:1365-1369 (1999); Latchman, et al., Nature Immunol., 2:261-268 (2001); and Tamura, Blood, 97:1809-1816 (2001)), the expression profiles of the proteins are quite distinct. Expression of PD-L1 protein, although essentially not found in normal tissues other than macrophage-like cells, can be induced in a variety of tissues and cell types (Dong, et al., Nature Med., 5:1365-1369 (1999); and Ishida, et al., Immunol. Lett., 84:57-62 (2000)). In contrast, PD-L2 is expressed only in dendritic cells and monocytes.

It has been shown that both PD-L1 and PD-L2 bind to PD-1 (Freeman, et al., J. Exp. Med., 192:1027-1034 (2000)), a distant member of the CD28 family with an immunoreceptor tyrosine-based inhibitory motif (ITIM) in its cytoplasmic domain (Ishida, et al., EMBO J., 11:3887-3895 (1992)). PD-1 is expressed on a subset of thymocytes and up-regulated on T, B, and myeloid cells after activation (Agata, et al., Int. Immunol., 8:765-772 (1996)). PD-1 acts to antagonize signal transduction downstream of the TCR after it binds a peptide antigen presented by the major histocompatibility complex (MHC). PD-1 signaling is thought to require binding to a ligand in close proximity to TCR:MHC complex, which occurs at the immunological synapse between a T cell and an antigen presenting cell (Freeman, Proc. Natl. Acad. Sci. U.S.A, 105:10275-10276 (2008)). The primary result of PD-1 ligation by its ligands is to inhibit signaling downstream of the TCR. Therefore, signal transduction via PD-1 usually provides a suppressive or inhibitory signal to the T cell that results in decreased T cell proliferation or other reduction in T cell activity.

The phenotypes of PD-1^{-/-} mice provide direct evidence for PD-1 being a negative regulator of immune responses in vivo. In the absence of PD-1, mice on the C57BL/6 background slowly develop a lupus-like glomerulonephritis and progressive arthritis (Nishimura, et al., Immunity, 11:141-151 (1999)). PD-1^{-/-} mice on the BALB/c background rapidly develop a fatal autoimmune dilated cardiomyopathy (Nishimura, et al., Science. 291:319-322 (2001)). However, substantial evidence indicates that PD-L2 can function to activate T cell responses. In the presence of suboptimal TCR signals, PD-L2 stimulates increased proliferation and production of cytokines in vitro (Tseng, et al., J. Exp. Med. 193:839-846 (2001)). On the other hand, in vitro studies indicate a negative regulatory role for PD-L2 in T cell responses. These seemingly contradictory data are best interpreted by expression of additional receptors for PD-L2 on T cells other than PD-1.

PD-L1 is the predominant PD-1 ligand causing inhibitory signal transduction in T cells. As PD-1 signaling is thought to require binding to a PD-1 ligand (typically PD-L1) in close proximity to the TCR:MHC complex, proteins, antibodies or small molecules that block the PD-1 receptor from interacting with its endogenous ligands, either by blocking the receptor or inhibiting its ligands, and thus prevent co-ligation of PD-1 and TCR on the T cell membrane are useful PD-1 antagonists that are contemplated.

It is therefore an object of the present invention to provide compositions and methods for inhibiting signal transduction through PD-1 on T cells.

It is another object of the invention to provide PD-1 antagonists that bind PD-1 but do not activate PD-1 signal transduction.

It is another object of the invention to provide PD-1 antagonists that bind to PD-1 ligands and inhibit or reduce the interaction of the ligand with PD-1.

It is another object of the invention to provide PD-1 antagonists that bind to the PD-1 receptor without engaging in signal transduction through the PD-1 receptor, bind to ligands of PD-1 and inhibit or reduce the interaction of the ligand with PD-1 receptors, and optionally activate T cells through a separate receptor pathway.

It is another object of the present invention to provide cells containing vectors that express nucleic acid molecules encoding antagonist polypeptides of PD-1.

It is a still further an object of the present invention to provide methods for enhancing and/or prolonging activation of T cells or overcoming T cell exhaustion and/or T cell anergy by contacting them with polypeptides that bind PD-1 without activating PD-1, or bind to ligands of PD-1 and inhibit or reduce the interaction of the ligand with PD-1 receptors.

It is still a further object of the invention to provide methods for administering antagonist polypeptides of PD-1, nucleic acids encoding the same, or cells transfected or transduced with nucleic acids encoding antagonist polypeptides of PD-1 to a mammal in need thereof.

It is still a further object of the invention to provide methods for potentiating an immune response to an antigen or a vaccine by administering antagonist polypeptides of PD-1 in combination with the antigen or vaccine.

### SUMMARY OF THE INVENTION

Compositions and methods for enhancing and/or prolonging the activation of T cells (i.e., increasing antigen-specific proliferation of T cells, enhancing cytokine production by T cells, stimulating differentiation ad effector functions of T cells and/or promoting T cell survival) or overcoming T cell exhaustion and/or anergy are provided. Representative compositions include PD-1 antagonists that bind to and block endogenous PD-1 on immune cells without triggering inhibitory signals from PD-1. In other embodiments, the compositions include PD-1 antagonists that bind to and block PD-1 ligands and thereby prevent them from interacting with PD-1. Methods for using the PD-1 antagonists to enhance immune responses in subjects in need thereof are provided.

PD-1 antagonists that bind to and block endogenous PD-1 on immune cells, preferably T cells, include PD-L1 and PD-L2 polypeptides, PD-1-binding fragments thereof, PD-1 antibodies, fusion proteins, and variants thereof. These PD-1 antagonist bind to PD-1 under physiological conditions and block T cell inhibition.

PD-1 antagonists that bind to native PD-1 ligands include PD-1 and B7.1 polypeptides, fragments thereof, antibodies, and fusion proteins. These PD-1 antagonists bind to B7-H1 and B7-DC and prevent them from triggering inhibitory signal transduction through PD-1 on immune cells.

In a preferred embodiment, B7-DC and B7-H1 polypeptides, or variants thereof are coupled to other polypeptides to form fusion proteins that antagonize the PD-1 receptor by binding to the PD-1 receptor without causing signal transduction through PD-1. Typically, the fusion polypeptides have a first fusion partner having all or a part of B7-DC or B7-H1, or variants thereof fused (i) directly to a second polypeptide or, (ii) optionally, fused to a linker peptide sequence that is fused to the second polypeptide. The presence of the fusion partner can alter the solubility, affinity and/or valency of the polypeptide. In certain embodiments, B7-DC, B7-H1 or variants thereof are fused to one or more domains of an Ig heavy chain constant region, preferably having an amino acid sequence corresponding to the hinge, C_{H2} and C_{H3} regions of a human immunoglobulin Cγ1 chain. Similar fusion proteins using B7.1 and PD-1 are provided.

Nucleic acids encoding PD-1 receptor antagonist polypeptides and fusion proteins and host cells containing such nucleic acids in vectors are also provided.

Immunogenic compositions containing the disclosed PD-1 receptor antagonists are also provided. Immunogenic compositions include antigens, a source of PD-1 receptor antagonist and optionally adjuvants. Suitable antigens include viral, bacterial, parasite, environmental and tumor antigens.

Methods for using PD-1 receptor antagonists to reduce T cell inhibition and/or prolong activation of T cells or overcome T cell exhaustion and/or anergy are provided. Therapeutic uses of PD-1 receptor antagonists and nucleic acids encoding the same are provided. PD-1 receptor antagonist compositions can be used to enhance immune responses to cancer. PD-1 receptor antagonist compositions can also be used to stimulate the immune response of immunosuppressed subjects. In certain embodiments, PD-1 receptor antagonist compositions are administered in conjunction with vaccines.

### BRIEF DESCRIPTION OF THE DRAWINGS

FIGS. 1A-B are graphs showing B7-DC-Ig binding to PD-1 in a PD-1 binding ELISA as described in Example 1.

FIG. 2 is a graph showing that B7-DC-Ig binds to PD-1 expressing CHO cells.

FIG. 3 is a graph showing that B7-DC-Ig competes with PD-L1 for binding to PD-1.

FIG. 4 is a diagram of an exemplary dosing regimen for the P815 tumor model.

FIGS. 5A-C are line graphs of tumor volumes plotted as a function of time and treatment: A) vehicle control, B) mouse IgG control, and C) murine B7-DC-Ig.

FIGS. 6A-C are line graphs showing that the combination of cyclophosphamide (CTX) and B7-DC-Ig resulted in eradication of established CT26 tumors (colon carcinoma) in mice.

FIG. 7 shows that the combination of CTX and B7-DC-Ig eradicated established CT26 tumors (colon carcinoma) in mice and protected against re-challenge with CT26.

FIG. 8 shows that CTX and B7-DC-Ig treatment resulted in generation of tumor specific memory CTLs.

### DETAILED DESCRIPTION OF THE INVENTION

### I. Definitions

As used herein the term “isolated” is meant to describe a compound of interest (e.g., either a polynucleotide or a polypeptide) that is in an environment different from that in which the compound naturally occurs e.g. separated from its natural milieu such as by concentrating a peptide to a concentration at which it is not found in nature. “Isolated” is meant to include compounds that are within samples that are substantially enriched for the compound of interest and/or in which the compound of interest is partially or substantially purified.

As used herein, the term “polypeptide” refers to a chain of amino acids of any length, regardless of modification (e.g., phosphorylation or glycosylation).

As used herein, a “variant” polypeptide contains at least one amino acid sequence alteration as compared to the amino acid sequence of the corresponding wild-type polypeptide.

As used herein, an “amino acid sequence alteration” can be, for example, a substitution, a deletion, or an insertion of one or more amino acids.

As used herein, a “vector” is a replicon, such as a plasmid, phage, or cosmid, into which another DNA segment may be inserted so as to bring about the replication of the inserted segment. The vectors described herein can be expression vectors.

As used herein, an “expression vector” is a vector that includes one or more expression control sequences

As used herein, an “expression control sequence” is a DNA sequence that controls and regulates the transcription and/or translation of another DNA sequence.

As used herein, “operably linked” means incorporated into a genetic construct so that expression control sequences effectively control expression of a coding sequence of interest.

As used herein, a “fragment” of a polypeptide refers to any subset of the polypeptide that is a shorter polypeptide of the full length protein. Generally, fragments will be five or more amino acids in length.

As used herein, “valency” refers to the number of binding sites available per molecule.

As used herein, “conservative” amino acid substitutions are substitutions wherein the substituted amino acid has similar structural or chemical properties.

As used herein, “non-conservative” amino acid substitutions are those in which the charge, hydrophobicity, or bulk of the substituted amino acid is significantly altered.

As used herein, “isolated nucleic acid” refers to a nucleic acid that is separated from other nucleic acid molecules that are present in a mammalian genome, including nucleic acids that normally flank one or both sides of the nucleic acid in a mammalian genome.

As used herein with respect to nucleic acids, the term “isolated” includes any non-naturally-occurring nucleic acid sequence, since such non-naturally-occurring sequences are not found in nature and do not have immediately contiguous sequences in a naturally-occurring genome.

As used herein, the term “host cell” refers to prokaryotic and eukaryotic cells into which a recombinant expression vector can be introduced.

As used herein, “transformed” and “transfected” encompass the introduction of a nucleic acid (e.g., a vector) into a cell by a number of techniques known in the art.

As used herein, the term “antibody” is meant to include both intact molecules as well as fragments thereof that include the antigen-binding site. These include Fab and F(ab′)₂ fragments which lack the Fc fragment of an intact antibody.

The terms “individual”, “host”, “subject”, and “patient” are used interchangeably herein, and refer to a mammal, including, but not limited to, humans, rodents, such as mice and rats, and other laboratory animals.

As used herein the term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being administered.

### II. PD-1 Antagonists

A preferred PD-1 antagonist compound for interfering with the interaction between PD-1 and PD-L1 is PD-L2 (also known as B7-DC), the extracellular domain of PD-L2, fusion proteins of PD-L2, and variants thereof which bind to and block PD-1 without triggering inhibitory signal transduction through PD-1, and prevent binding of PD-L1 to PD-1. Additional PD-1 antagonists include fragments of PD-L1 that bind to PD-1 without triggering inhibitory signal transduction through PD-1, PD-1 or soluble fragments thereof that bind to ligands of PD-1 and prevent binding to the endogenous PD-1 receptor on T cells, and B7.1 or soluble fragments thereof that can bind to PD-L1 and prevent binding of PD-L1 to PD-1. In certain embodiments, PD-1 antagonists increase T cell cytotoxicity in a subject. The multiple functionality PD-1 antagonists helps to induce a robust immune response in subjects and overcome T cell exhaustion and T cell anergy.

PD-1 antagonists bind to ligands of PD-1 and interfere with or inhibit the binding of the ligands to the PD-1 receptor, or bind directly to the PD-1 receptor without engaging in signal transduction through the PD-1 receptor. In preferred embodiments, the PD-1 antagonists bind directly to PD-1 and block PD-1 inhibitory signal transduction. In other embodiments the PD-1 antagonists bind to ligands of PD-1 and reduce or inhibit the ligands from triggering inhibitory signal transduction through the PD-1. In still another embodiment, the PD-1 antagonists can activate T cells by binding to a receptor other than the PD-1 receptor.

The PD-1 antagonists can be small molecule antagonists. The term “small molecule” refers to small organic compounds having a molecular weight of more than 100 and less than about 2,500 daltons, preferably between 100 and 2000, more preferably between about 100 and about 1250, more preferably between about 100 and about 1000, more preferably between about 100 and about 750, more preferably between about 200 and about 500 daltons. The small molecules often include cyclical carbon or heterocyclic structures and/or aromatic or polyaromatic structures substituted with one or more functional groups. The small molecule antagonists reduce or interfere with PD-1 receptor signal transduction by binding to ligands of PD-1 such as PD-L1 and PD-L2 and preventing the ligand from interacting with PD-1 or by binding directly to the PD-1 receptor without triggering signal transduction through the PD-1 receptor.

Exemplary PD-1 antagonists include, but are not limited to, PD-L2, PD-L1, PD-1 or B7-1 polypeptides, and variants, fragments or fusion proteins thereof. Additional embodiments include antibodies that bind to any of these proteins.

A. PD-L2 Based PD-1 Antagonists

1. PD-L2 Based PD-1 Antagonists that Bind to PD-1

PD-1 antagonists bind to PD-1 on immune cells and block inhibitory PD-1 signaling. PD-1 signal transduction is thought to require binding to PD-1 by a PD-1 ligand (PD-L2 or PD-L1; typically PD-L1) in close proximity to the TCR:MHC complex within the immune synapse. Therefore, proteins, antibodies or small molecules that block inhibitory signal transduction through PD-1 and optionally prevent co-ligation of PD-1 and TCR on the T cell membrane are useful PD-1 antagonists.

Representative polypeptide antagonists include, but are not limited to, PD-L2 polypeptides, fragments thereof, fusion proteins thereof, and variants thereof. PD-L2 polypeptides that bind to PD-1 and block inhibitory signal transduction through PD-1 are one of the preferred embodiments. Other embodiments include PD-1 antagonists that prevent native ligands of PD-1 from binding and triggering signal transduction. In certain embodiments, it is believed that the disclosed PD-L2 polypeptides have reduced or no ability to trigger signal transduction through the PD-1 receptor because there is no co-ligation of the TCR by the peptide-MHC complex in the context of the immune synapse. Because signal transduction through the PD-1 receptor transmits a negative signal that attenuates T-cell activation and T-cell proliferation, inhibiting the PD-1 signal transduction pathway allows cells to be activated that would otherwise be attenuated.

2. Exemplary PD-L2 Polypeptide Antagonists

Murine PD-L2 polypeptides can have at least 80%, 85%, 90%, 95%, 99% or 100% sequence identity to:

| (SEQ ID NO: 1) | |
|---|---|
| MLLLLPILNL SLQLHPVAAL FTVTAPKEVY TVDVGSSVSL ECDFDRRECT ELEGIRASLQ | 60 |
| KVENDTSLQS ERATLLEEQL PLGKALFHIP SVQVRDSGQY RCLVICGAAW DYKYLTVKVK | 120 |
| ASYMRIDTRI LEVPGTGEVQ LTCQARGYPL AEVSWQNVSV PANTSHIRTP EGLYQVTSVL | 180 |
| RLKPQPSRNF SCMFWNAHMK ELTSAIIDPL SRMEPKVPRT WPLHVFIPAC TIALIFLAIV | 240 |
| IIQRKRI | 247 |
| or | |
| (SEQ ID NO: 2) | |
| LFTVTAPKEV YTVDVGSSVS LECDFDRREC TELEGIRASL QKVENDTSLQ SERATLLEEQ | 60 |
| LPLGKALFHI PSVQVRDSGQ YRCLVICGAA WDYKYLTVKV KASYMRIDTR ILEVPGTGEV | 120 |
| QLTCQARGYP LAEVSWQNVS VPANTSHIRT PEGLYQVTSV LRLKPQPSRN FSCMFWNAHM | 180 |
| KELTSAIIDP LSRMEPKVPR TWPLHVFIPA CTIALIFLAI VIIQRKRI. | 228 |

Human PD-L2 polypeptides can have at least 80%, 85%, 90%, 95%, 99% or 100% sequence identity to:

| (SEQ ID NO: 3) | |
|---|---|
| MIFLLLMLSL ELQLHQIAAL FTVTVPKELY IIEHGSNVTL ECNFDTGSHV NLGAITASLQ | 60 |
| KVENDTSPHR ERATLLEEQL PLGKASFHIP QVQVRDEGQY QCIIIYGVAW DYKYLTLKVK | 120 |
| ASYRKINTHI LKVPETDEVE LTCQATGYPL AEVSWPNVSV PANTSHSRTP EGLYQVTSVL | 180 |
| RLKPPPGRNF SCVFWNTHVR ELTLASIDLQ SQMEPRTHPT WLLHIFIPFC IIAFIFIATV | 240 |
| IALRKQLCQK LYSSKDTTKR PVTTTKREVN SAI | 273 |
| or | |
| (SEQ ID NO: 4) | |
| LFTVTVPKEL YIIEHGSNVT LECNFDTGSH VNLGAITASL QKVENDTSPH RERATLLEEQ | 60 |
| LPLGKASFHI PQVQVRDEGQ YQCIIIYGVA WDYKYLTLKV KASYRKINTH ILKVPETDEV | 120 |
| ELTCQATGYP LAEVSWPNVS VPANTSHSRT PEGLYQVTSV LRLKPPPGRN FSCVEWNTHV | 180 |
| RELTLASIDL QSQMEPRTHP TWLLHIFIPF CIIAFIFIAT VIALRKQLCQ KLYSSKDTTK | 240 |
| RPVTTTKREV NSAI | 254. |

Non-human primate (Cynomolgus) PD-L2 polypeptides can have at least 80%, 85%, 90%, 95%, 99% or 100% sequence identity to:

| (SEQ ID NO: 5) | |
|---|---|
| MIFLLLMLSL ELQLHQIAAL FTVTVPKELY IIEHGSNVTL ECNFDTGSHV NLGAITASLQ | 60 |
| KVENDTSPHR ERATLLEEQL PLGKASFHIP QVQVRDEGQY QCIIIYGVAW DYKYLTLKVK | 120 |
| ASYRKINTHI LKVPETDEVE LTCQATGYPL AEVSWPNVSV PANTSHSRTP EGLYQVTSVL | 180 |
| RLKPPPGRNF SCVFWNTHVR ELTLASIDLQ SQMEPRTHPT WLLHIFIPSC IIAFIFIATV | 240 |
| IALRKQLCQK LYSSKDATKR PVTTTKREVN SAI | 273 |
| or | |
| (SEQ ID NO: 6) | |
| LFTVTVPKEL YIIEHGSNVT LECNFDTGSH VNLGAITASL QKVENDTSPH RERATLLEEQ | 60 |
| LPLGKASFHI PQVQVRDEGQ YQCIIIYGVA WDYKYLTLKV KASYRKINTH ILKVPETDEV | 120 |
| ELTCQATGYP LAEVSWPNVS VPANTSHSRT PEGLYQVTSV LRLKPPPGRN FSCVFWNTHV | 180 |
| RELTLASIDL QSQMEPRTHP TWLLHIFIPS CIIAFIFIAT VIALRKQLCQ KLYSSKDATK | 240 |
| RPVTTTKREV NSAI | 254 |

SEQ ID NOs: 1, 3 and 5 each contain a signal peptide.

B. PD-L1 Based PD-1 Antagonists

1. PD-L1 Based PD-1 Antagonists that Bind to PD-1 Receptors

Other PD-1 antagonists that bind to the PD-1 receptor include, but are not limited to, PD-L1 polypeptides, fragments thereof, fusion proteins thereof, and variants thereof. These PD-1 polypeptide antagonists bind to and block the PD-1 receptor and have reduced or no ability to trigger inhibitory signal transduction through the PD-1 receptor. In one embodiment, it is believed that the PD-L1 polypeptides have reduced or no ability to trigger signal transduction through the PD-1 receptor because there is no co-ligation of the TCR by the peptide-MHC complex in the context of the immune synapse. Because signal transduction through the PD-1 receptor transmits a negative signal that attenuates T-cell activation and T-cell proliferation, inhibiting the PD-1 signal transduction using PD-L1 polypeptides allows cells to be activated that would otherwise be attenuated.

2. Exemplary PD-L1 Polypeptide PD-1 Antagonists

Murine polypeptides can have at least 80%, 85%, 90%, 95%, 99% or 100% sequence identity to:

| (SEQ ID NO: 7) | |
|---|---|
| MRIFAGIIFT ACCHLLRAFT ITAPKDLYVV EYGSNVTMEC RFPVERELDL LALVVYWEKE | 60 |
| DEQVIQFVAG EEDLKPQHSN FRGRASLPKD QLLKGNAALQ ITDVKLQDAG VYCCIISYGG | 120 |
| ADYKRITLKV NAPYRKINQR ISVDPATSEH ELICQAEGYP EAEVIWTNSD HQPVSGKRSV | 180 |
| TTSRTEGMLL NVTSSLRVNA TANDVFYCTF WRSQPGQNHT AELIIPELPA THPPQNRTHW | 240 |
| VLLGSILLFL IVVSTVLLFL RKQVRMLDVE KCGVEDTSSK NRNDTQEEET | 290 |
| or | |
| (SEQ ID NO: 8) | |
| FTITAPKDLY VVEYGSNVTM ECRFPVEREL DLLALVVYWE KEDEQVIQFV AGEEDLKPQH | 60 |
| SNFRGRASLP KDQLLKGNAA LQITDVKLQD AGVYCCIISY GGADYKRITL KVNAPYRKIN | 120 |
| QRISVDPATS EHELICQAEG YPEAEVIWTN SDHQPVSGKR SVTTSRTEGM LLNVTSSLRV | 180 |
| NATANDVFYC TFWRSQPGQN HTAELIIPEL PATHPPQNRT HWVLLGSILL FLIVVSTVLL | 240 |
| FLRKQVRMLD VEKCGVEDTS SKNRNDTQFE ET | 272. |

Human PD-L1 polypeptides can have at least 80%, 85%, 90%, 95%, 99% or 100% sequence identity to:

| (SEQ ID NO: 9) | |
|---|---|
| MRIFAVFIFM TYWHLLNAFT VTVPKDLYVV EYGSNMTIEC KFPVEKQLDL AALIVYWEME | 60 |
| DKNIIQFVHG EEDLKVQHSS YRQRARLLKD QLSLGNAALQ ITDVKLQDAG VYRCMISYGG | 120 |
| ADYKRITVKV NAPYNKINQR ILVVDPVTSE HELTCQAEGY PKAEVIWTSS DHQVLSGKTT | 180 |
| TTNSKREEKL FNVTSTLRIN TTTNEIFYCT FRRLDPEENH TAELVIPELP LAHPPNERTH | 240 |
| LVILGAILLC LGVALTFIFR LRKGRMMDVK KCGIQDTNSK KQSDTHLEET | 290 |
| or | |
| (SEQ ID NO: 10) | |
| FTVTVPKDLY VVEYGSNMTI ECKFPVEKQL DLAALIVYWE MEDKNIIQFV HGEEDLKVQH | 60 |
| SSYRQRARLL KDQISLGNAA LQITDVKLQD AGVYRCMISY GGADYKRITV KVNAPYNKIN | 120 |
| QRILVVDPVT SEHELTCQAE GYPKAEVIWT SSDHQVLSGK TTTTNSKREE KLFNVTSTLR | 180 |
| INTTTNEIFY CTFRRLDPEE NHTAELVIPE LPLAHPPNER THLVILGAIL LCLGVALTFI | 240 |
| FRLRKGRMMD VKKCGIQDTN SKKQSDTHLE ET | 272. |

SEQ ID NOs: 7 and 9 each contain a signal peptide.

C. B7.1 and PD-1 Based PD-1 Antagonists

1. B7.1 and PD-1 Based PD-1 Antagonists that Bind to PD-L1 and PD-L2

Other useful polypeptides include the PD-1 receptor protein, or soluble fragments thereof, which can bind to the PD-1 ligands, such as PD-L1 or PD-L2, and prevent binding to the endogenous PD-1 receptor, thereby preventing inhibitory signal transduction. Such fragments also include the soluble ECD portion of the PD-1 protein that optionally includes mutations, such as the A99L mutation, that increases binding to the natural ligands. PD-L1 has also been shown to bind the protein B7.1 (Butte, et al., Immunity, 27(1): 111-122 (2007)). Therefore, B7.1 or soluble fragments thereof, which can bind to the PD-L1 ligand and prevent binding to the endogenous PD-1 receptor, thereby preventing inhibitory signal transduction, are also useful.

2. Exemplary B7.1 Polypeptide PD-1 Antagonists

Murine B7.1 polypeptides can have at least 80%, 85%, 90%, 95%, 99% or 100% sequence identity to:

| (SEQ ID NO: 11) | |
|---|---|
| MACNCQLMQD TPLLKFPCPR LILLFVLLIR LSQVSSDVDE QLSKSVKDKV LLPCRYNSPH | 60 |
| EDESEDRIYW QKHDKVVLSV IAGKLKVWPE YKNRTLYDNT TYSLIILGLV LSDRGTYSCV | 120 |
| VQKKERGTYE VKHLALVKLS IKADFSTPNI TESGNPSADT KRITCFASGG FPKPRFSWLE | 180 |
| NGRELPGINT TISQDPESEL YTISSQLDFN TTRNHTIKCL IKYGDAHVSE DFTWEKPPED | 240 |
| PPDSKNTLVL FGAGFGAVIT VVVIVVIIKC FCKHRSCFRR NEASRETNNS LTFGPEEALA | 300 |
| EQTVFL | 306 |
| or | |
| (SEQ ID NO: 12) | |
| VDEQLSKSVK DKVLLPCRYN SPHEDESEDR IYWQKHDKVV LSVIAGKLKV WPEYKNRTLY | 60 |
| DNTTYSLIIL GLVLSDRGTY SCVVQKKERG TYEVKHLALV KLSIKADFST PNITESGNPS | 120 |
| ADTKRITCFA SGGFPKPRFS WLENGRELPG INTTISQDPE SELYTISSQL DFNTTRNHTI | 180 |
| KCLIKYGDAH VSEDFTWEKP PEDPPDSKNT LVLFGAGFGA VITVVVIVVI IKCFCKHRSC | 240 |
| FRRNEASRET NNSLTFGPEE ALAEQTVFL | 269. |

Human B7.1 polypeptides can have at least 80%, 85%, 90%, 95%, 99% or 100% sequence identity to:

| (SEQ ID NO: 13) | |
|---|---|
| MGHTRRQGTS PSKCPYLNFF QLLVLAGLSH FCSGVIHVTK EVKEVATLSC GHNVSVEELA | 60 |
| QTRIYWQKEK KMVLTMMSGD MNIWPEYKNR TIFDITNNLS IVILALRPSD EGTYECVVLK | 120 |
| YEKDAFKREH LAEVTLSVKA DFPTPSISDF EIPTSNIRRI ICSTSGGFPE PHLSWLENGE | 180 |
| ELNAINTTVS QDPETELYAV SSKLDFNMTT NHSFMCLIKY GHLRVNQTFN WNTTKQEHFP | 240 |
| DNLLPSWAIT LISVNGIFVI CCLTYCFAPR CRERRRNERL RRESVRPV | 288 |
| or | |
| (SEQ ID NO: 14) | |
| VIHVTKEVKE VATLSCGHNV SVEELAQTRI YWQKEKKMVL TMMSGDMNIW PEYKNRTIFD | 60 |
| ITNNLSIVIL ALRPSDEGTY ECVVLKYEKD AFKREHLAEV TLSVKADFPT PSISDFEIPT | 120 |
| SNIRRIICST SGGFPEPHLS WLENGEELNA INTTVSQDPE TELYAVSSKL DFNMTTNHSF | 180 |
| MCLIKYGHLR VNQTFNWNTT KQEHFPDNLL PSWAITLISV NGIFVICCLT YCFAPRCRER | 240 |
| RRNERLRRES VRPV | 254. |

SEQ ID NOs: 11 and 13 each contain a signal peptide.

3. Exemplary PD-1 Polypeptide PD-1 Antagonists

Human PD-1 polypeptides can have at least 80%, 85%, 90%, 95%, 99% or 100% sequence identity to:

| (SEQ ID NO: 15) | |
|---|---|
| MQIPQAPWPV VWAVLQLGWR PGWFLDSPDR PWNPPTFFPA LLVVTEGDNA TFTCSFSNTS | 60 |
| ESFVLNWYRM SPSNQTDKLA AFPEDRSQPG QDCRFRVTQL PNGRDFHMSV VRARRNDSGT | 120 |
| YLCGAISLAP KAQIKESLRA ELRVTERRAE VPTAHPSPSP RPAGQFQTLV VGVVGGLLGS | 180 |
| LVLLVWVLAV ICSRAARGTI GARRTGQPLK EDPSAVPVFS VDYGELDFQW REKTPEPPVP | 240 |
| CVPEQTEYAT IVFPSGMGTS SPARRGSADG PRSAQPLRPE DGHCSWPL | 288 |

Non-human primate (Cynomolgus) PD-1 polypeptides can have at least 80%, 85%, 90%, 95%, 99% or 100% sequence identity to:

| (SEQ ID NO: 16) | |
|---|---|
| MQIPQAPWPV VWAVLQLGWR PGWFLESPDR PWNAPTFSPA LLLVTEGDNA TFTCSFSNAS | 60 |
| ESFVLNWYRM SPSNQTDKLA AFPEDRSQPG QDCRFRVTRL PNGRDFHMSV VRARRNDSGT | 120 |
| YLCGAISLAP KAQIKESLRA ELRVTERRAE VPTAHPSPSP RPAGQFQTLV VGVVGGLLGS | 180 |
| LVLLVWVLAV ICSRAARGTI GARRTGQPLK EDPSAVPVFS VDYGELDFQW REKTPEPPVP | 240 |
| CVPEQTEYAT IVFPSGMGTS SPARRGSADG PRSAQPLRPE DGHCSWPL | 288 |

SEQ ID NOs: 15 and 16 each contain a signal peptide.

D. Fragments of PD-1 Antagonist Polypeptides

The PD-1 antagonist polypeptides can be full-length polypeptides, or can be a fragment of a full length polypeptide. As used herein, a fragment of a PD-1 antagonist polypeptide refers to any subset of the polypeptide that is a shorter polypeptide of the full length protein.

Useful fragments are those that retain the ability to bind to their natural ligands. A PD-1 antagonist polypeptide that is a fragment of full-length PD-1 antagonist polypeptide typically has at least 20 percent, 30 percent, 40 percent, 50 percent, 60 percent, 70 percent, 80 percent, 90 percent, 95 percent, 98 percent, 99 percent, 100 percent, or even more than 100 percent of the ability to bind its natural ligand(s) as compared to the full-length PD-1 antagonist polypeptide.

For example, useful fragments of PD-L2 and PD-L1 are those that retain the ability to bind to PD-1. PD-L2 and PD-L1 fragments typically have at least 20 percent, 30 percent, 40 percent, 50 percent, 60 percent, 70 percent, 80 percent, 90 percent, 95 percent, 98 percent, 99 percent, 100 percent, or even more than 100 percent of the ability to bind to PD-1 as compared to full length PD-L2 and PD-L1.

Fragments of PD-1 antagonist polypeptides include soluble fragments. Soluble PD-1 antagonist polypeptide fragments are fragments of PD-1 antagonist polypeptides that may be shed, secreted or otherwise extracted from the producing cells. Soluble fragments of PD-1 antagonist polypeptides include some or all of the extracellular domain of the polypeptide, and lack some or all of the intracellular and/or transmembrane domains. In one embodiment, PD-1 antagonist polypeptide fragments include the entire extracellular domain of the PD-1 antagonist polypeptide. It will be appreciated that the extracellular domain can include 1, 2, 3, 4, or 5 amino acids from the transmembrane domain. Alternatively, the extracellular domain can have 1, 2, 3, 4, or 5 amino acids removed from the C-terminus, N-terminus, or both.

Generally, the PD-1 antagonist polypeptides or fragments thereof are expressed from nucleic acids that include sequences that encode a signal sequence. The signal sequence is generally cleaved from the immature polypeptide to produce the mature polypeptide lacking the signal sequence. The signal sequence of PD-1 antagonist polypeptides can be replaced by the signal sequence of another polypeptide using standard molecule biology techniques to affect the expression levels, secretion, solubility, or other property of the polypeptide. The signal sequence that is used to replace the PD-1 antagonist polypeptide signal sequence can be any known in the art.

1. PD-L2 Extracellular Domains

a. Human PD-L2 Extracellular Domains

In one embodiment, the PD-1 antagonist polypeptide includes the extracellular domain of human PD-L2 or a fragment thereof. The PD-1 antagonist polypeptide can be encoded by a nucleotide sequence having at least 80%, 85%, 90%, 95%, 99%, or 100% sequence identity to:

| (SEQ ID NO: 17) | |
|---|---|
| atgatctttc ttctcttgat gctgtctttg gaattgcaac ttcaccaaat cgcggccctc | 60 |
| tttactgtga ccgtgccaaa agaactgtat atcattgagc acgggtccaa tgtgaccctc | 120 |
| gaatgtaact ttgacaccgg cagccacgtt aacctggggg ccatcactgc cagcttgcaa | 180 |
| aaagttgaaa acgacacttc acctcaccgg gagagggcaa ccctcttgga ggagcaactg | 240 |
| ccattgggga aggcctcctt tcatatccct caggtgcagg ttcgggatga gggacagtac | 300 |
| cagtgcatta ttatctacgg cgtggcttgg gattacaagt atctgaccct gaaggtgaaa | 360 |
| gcgtcctatc ggaaaattaa cactcacatt cttaaggtgc cagagacgga cgaggtggaa | 420 |
| ctgacatgcc aagccaccgg ctacccgttg gcagaggtca gctggcccaa cgtgagcgta | 480 |
| cctgctaaca cttctcattc taggacaccc gagggcctct accaggttac atccgtgctc | 540 |
| cgcctcaaac cgcccccagg ccggaatttt agttgcgtgt tttggaatac ccacgtgcga | 600 |
| gagctgactc ttgcatctat tgatctgcag tcccagatgg agccacggac tcatccaact | 660 |
| tgg | 663. |

In another embodiment, the PD-1 antagonist polypeptide can have at least 80%, 85%, 90%, 95%, 99%, or 100% sequence identity to the human amino acid sequence:

| (SEQ ID NO: 18) | |
|---|---|
| MIFLLLMLSL ELQLHQIAAL FTVTVPKELY IIEHGSNVTL | |
| MIFLLLMLSL ELQLHQIAAL FTVTVPKELY IIEHGSNVTL ECNFDTGSHV NLGAITASLQ | 60 |
| KVENDTSPHR ERATLLEEQL PLGKASFHIP QVQVRDEGQY QCIIIYGVAW DYKYLTLKVK | 120 |
| ASYRKINTHI LKVPETDEVE LTCQATGYPL AEVSWPNVSV PANTSHSRTP EGLYQVTSVL | 180 |
| RLKPPPGRNF SCVFWNTHVR ELTLASIDLQ SQMEPRTHPT W | 221. |

It will be appreciated that the signal sequence will be removed in the mature protein. Additionally, it will be appreciated that signal peptides from other organisms can be used to enhance the secretion of the protein from a host during manufacture. SEQ ID NO:19 provides the human amino acid sequence of SEQ ID NO:18 without the signal sequence:

| (SEQ ID NO: 19) | |
|---|---|
| LFTVTVPKEL YIIEHGSNVT LECNFDTGSH VNLGAITASL QKVENDTSPH RERATLLEEQ | 60 |
| LPLGKASFHI PQVQVRDEGQ YQCIIIYGVA WDYKYLTLKV KASYRKINTH ILKVPETDEV | 120 |
| ELTCQATGYP LAEVSWPNVS VPANTSHSRT PEGLYQVTSV LRLKPPPGRN FSCVFWNTHV | 180 |
| RELTLASIDL QSQMEPRTHP TW | 202. |

In another embodiment, the PD-1 antagonist polypeptide includes the IgV domain of human PD-L2. The first fusion partner can be encoded by a nucleotide sequence having at least 80%, 85%, 90%, 95%, 99%, or 100% sequence identity to:

| (SEQ ID NO: 20) | |
|---|---|
| tttactgtga ccgtgccaaa agaactgtat atcattgagc acgggtccaa tgtgaccctc | 60 |
| gaatgtaact ttgacaccgg cagccacgtt aacctggggg ccatcactgc cagcttgcaa | 120 |
| aaagttgaaa acgacacttc acctcaccgg gagagggcaa ccctcttgga ggagcaactg | 180 |
| ccattgggga aggcctcctt tcatatccct caggtgcagg ttcgggatga gggacagtac | 240 |
| cagtgcatta ttatctacgg cgtggcttgg gattacaagt atctgaccct gaag | 294. |

The PD-1 antagonist polypeptide can have at least 80%, 85%, 90%, 95%, 99%, or 100% sequence identity to the human amino acid sequence:

| (SEQ ID NO: 21), also referred to as PD-L2V. | |
|---|---|
| FTVTVPKELY IIEHGSNVTL ECNFDTGSHV NLGAITASLQ KVENDTSPHR ERATLLEEQL | 60 |
| PLGKASFHIP QVQVRDEGQY QCIIIYGVAW DYKYLTLK | 98 |

b. Non-Human Primate PD-L2 Extracellular Domains

In one embodiment, the PD-1 antagonist polypeptide includes the extracellular domain of non-human primate (Cynomolgus) PD-L2 or a fragment thereof. The PD-1 antagonist polypeptide can be encoded by a nucleotide sequence having at least 80%, 85%, 90%, 95%, 99%, or 100% sequence identity to:

| (SEQ ID NO: 22) | |
|---|---|
| atgatcttcc tcctgctaat gttgagcctg gaattgcagc ttcaccagat agcagcttta | 60 |
| ttcacagtga cagtccctaa ggaactgtac ataatagagc atggcagcaa tgtgaccctg | 120 |
| gaatgcaact ttgacactgg aagtcatgtg aaccttggag caataacagc cagtttgcaa | 180 |
| aaggtggaaa atgatacatc cccacaccgt gaaagagcca ctttgctgga ggagcagctg | 240 |
| cccctaggga aggcctcgtt ccacatacct caagtccaag tgagggacga aggacagtac | 300 |
| caatgcataa tcatctatgg ggtcgcctgg gactacaagt acctgactct gaaagtcaaa | 360 |
| gcttcctaca ggaaaataaa cactcacatc ctaaaggttc cagaaacaga tgaggtagag | 420 |
| ctcacctgcc aggctacagg ttatcctctg gcagaagtat cctggccaaa cgtcagcgtt | 480 |
| cctgccaaca ccagccactc caggacccct gaaggcctct accaggtcac cagtgttctg | 540 |
| cgcctaaagc caccccctgg cagaaacttc agctgtgtgt tctggaatac tcacgtgagg | 600 |
| gaacttactt tggccagcat tgaccttcaa agtcagatgg aacccaggac ccatccaact | 660 |
| tgg | 663. |

In another embodiment, the PD-1 antagonist polypeptide can have at least 80%, 85%, 90%, 95%, 99%, or 100% sequence identity to the non-human primate amino acid sequence:

| (SEQ ID NO: 23) | |
|---|---|
| MIFLLLMLSL ELQLHQIAAL FTVTVPKELY IIEHGSNVTL ECNFDTGSHV NLGAITASLQ | 60 |
| KVENDTSPHR ERATLLEEQL PLGKASFHIP QVQVRDEGQY QCIIIYGVAW DYKYLTLKVK | 120 |
| ASYRKINTHI LKVPETDEVE LTCQATGYPL AEVSWPNVSV PANTSHSRTP EGLYQVTSVL | 180 |
| RLKPPPGRNF SCVFWNTHVR ELTLASIDLQ SQMEPRTHPT W | 221. |

The signal sequence will be removed in the mature protein. Additionally, signal peptides from other organisms can be used to enhance the secretion of the fusion protein from a host during manufacture. SEQ ID NO:24 provides the non-human primate amino acid sequence of SEQ ID NO:23 without the signal sequence:

(SEQ ID NO: 24)

| | |
|---|---|
| LFTVTVPKEL YIIEHGSNVT LECNFDTGSH VNLGAITASL QKVENDTSPH RERATLLEEQ | 60 |

| | |
|---|---|
| LPLGKASFHI PQVQVRDEGQ YQCIIIYGVA WDYKYLTLKV KASYRKINTH ILKVPETDEV | 120 |
| ELTCQATGYP LAEVSWPNVS VPANTSHSRT PEGLYQVTSV LRLKPPPGRN FSCVFWNTHV | 180 |
| RELTLASIDL QSQMEPRTHP TW. | 202 |

In another embodiment, the PD-1 antagonist polypeptide includes the IgV domain of non-human primate PD-L2. The first fusion partner can be encoded by a nucleotide sequence having at least 80%, 85%, 90%, 95%, 99%, or 100% sequence identity to:

(SEQ ID NO: 25)

| | |
|---|---|
| ttcacagtga cagtccctaa ggaactgtac ataatagagc atggcagcaa tgtgaccctg | 60 |

| | |
|---|---|
| gaatgcaact ttgacactgg aagtcatgtg aaccttggag caataacagc cagtttgcaa | 120 |
| aaggtggaaa atgatacatc cccacaccgt gaaagagcca ctttgctgga ggagcagctg | 180 |
| cccctaggga aggcctcgtt ccacatacct caagtccaag tgagggacga aggacagtac | 240 |
| caatgcataa tcatctatgg ggtcgcctgg gactacaagt acctgactct gaaa. | 294 |

The PD-1 antagonist polypeptide can have at least 80%, 85%, 90%, 95%, 99%, or 100% sequence identity to the non-human primate amino acid sequence:

| (SEQ ID NO: 26) | |
|---|---|
| FTVTVPKELY IIEHGSNVTL ECNFDTGSHV NLGAITASLQ KVENDTSPHR ERATLLEEQL | 60 |
| PLGKASFHIP QVQVRDEGQY QCIIIYGVAW DYKYLTLK, | 98 |

also referred to as PD-L2V.

d. Murine PD-L2 Extracellular Domains

In one embodiment, the PD-1 antagonist polypeptide includes the extracellular domain of murine PD-L2 or a fragment thereof. The PD-1 antagonist polypeptide can be encoded by a nucleotide sequence having at least 80%, 85%, 90%, 95%, 99%, or 100% sequence identity to:

| (SEQ ID NO: 27) | |
|---|---|
| atgctgctcc tgctgccgat actgaacctg agcttacaac ttcatcctgt agcagcttta | 60 |
| ttcaccgtga cagcccctaa agaagtgtac accgtagacg tcggcagcag tgtgagcctg | 120 |
| gagtgcgatt ttgaccgcag agaatgcact gaactggaag ggataagagc cagtttgcag | 180 |
| aaggtagaaa atgatacgtc tctgcaaagt gaaagagcca ccctgctgga ggagcagctg | 240 |
| cccctgggaa aggctttgtt ccacatccct agtgtccaag tgagagattc cgggcagtac | 300 |
| cgttgcctgg tcatctgcgg ggccgcctgg gactacaagt acctgacggt gaaagtcaaa | 360 |
| gcttcttaca tgaggataga cactaggatc ctggaggttc caggtacagg ggaggtgcag | 420 |
| cttacctgcc aggctagagg ttatccccta gcagaagtgt cctggcaaaa tgtcagtgtt | 480 |
| cctgccaaca ccagccacat caggaccccc gaaggcctct accaggtcac cagtgttctg | 540 |
| cgcctcaagc ctcagcctag cagaaacttc agctgcatgt tctggaatgc tcacatgaag | 600 |
| gagctgactt cagccatcat tgaccctctg agtcggatgg aacccaaagt ccccagaacg | 660 |
| tgg. | 663 |

In another embodiment, the PD-1 antagonist polypeptide can have at least 80%, 85%, 90%, 95%, 99%, or 100% sequence identity to the murine amino acid sequence:

| (SEQ ID NO: 28) | |
|---|---|
| MLLLLPILNL SLQLHPVAAL FTVTAPKEVY TVDVGSSVSL ECDFDRRECT ELEGIRASLQ | 60 |
| KVENDTSLQS ERATLLEEQL PLGKALFHIP SVQVRDSGQY RCLVICGAAW DYKYLTVKVK | 120 |
| ASYMRIDTRI LEVPGTGEVQ LTCQARGYPL AEVSWQNVSV PANTSHIRTP EGLYQVTSVL | 180 |
| RLKPQPSRNF SCMFWNAHMK ELTSAIIDPL SRMEPKVPRT W. | 221 |

The signal sequence will be removed in the mature protein. Additionally, signal peptides from other organisms can be used to enhance the secretion of the protein from a host during manufacture. SEQ ID NO:29 provides the murine amino acid sequence of SEQ ID NO:28 without the signal sequence:

| (SEQ ID NO: 29) | |
|---|---|
| LFTVTAPKEV YTVDVGSSVS LECDFDRREC TELEGIRASL QKVENDTSLQ SERATLLEEQ | 60 |
| LPLGKALFHI PSVQVRDSGQ YRCLVICGAA WDYKYLTVKV KASYMRIDTR ILEVPGTGEV | 120 |
| QLTCQARGYP LAEVSWQNVS VPANTSHIRT PEGLYQVTSV LRLKPQPSRN FSCMFWNAHM | 180 |
| KELTSAIIDP LSRMEPKVPR TW. | 202 |

In another embodiment, the PD-1 antagonist polypeptide includes the IgV domain of murine PD-L2. The first fusion partner can be encoded by a nucleotide sequence having at least 80%, 85%, 90%, 95%, 99%, or 100% sequence identity to:

| (SEQ ID NO: 30) | |
|---|---|
| ttcaccgtga cagcccctaa agaagtgtac accgtagacg tcggcagcag tgtgagcctg | 60 |
| gagtgcgatt ttgaccgcag agaatgcact gaactggaag ggataagagc cagtttgcag | 120 |
| aaggtagaaa atgatacgtc tctgcaaagt gaaagagcca ccctgctgga ggagcagctg | 180 |
| cccctgggaa aggctttgtt ccacatccct agtgtccaag tgagagattc cgggcagtac | 240 |
| cgttgcctgg tcatctgcgg ggccgcctgg gactacaagt acctgacggt gaaa. | 294 |

The PD-1 antagonist polypeptide can have at least 80%, 85%, 90%, 95%, 99%, or 100% sequence identity to the murine amino acid sequence:

| (SEQ ID NO: 31) | |
|---|---|
| FTVTAPKEVY TVDVGSSVSL ECDFDRRECT ELEGTRASLQ KVENDTSLQS ERATLLEEQL | 60 |
| PLGKALFHIP SVQVRDSGQY RCLVICGAAW DYKYLTVK, | 98 |

also referred to as PD-L2V.

d. PD-L2 Extracellular Domain Fragments

The PD-L2 extracellular domain can contain one or more amino acids from the signal peptide or the putative transmembrane domain of PD-L2. During secretion, the number of amino acids of the signal peptide that are cleaved can vary depending on the expression system and the host. Additionally, fragments of PD-L2 extracellular domain missing one or more amino acids from the C-terminus or the N-terminus that retain the ability to bind to PD-1 can be used.

Exemplary suitable fragments of murine PD-L2 that can be used as a first fusion partner include, but are not limited to, the following:

24-221, 24-220, 24-219, 24-218, 24-217, 24-216, 24-215,

23-221, 23-220, 23-219, 23-218, 23-217, 23-216, 23-215,

22-221, 22-220, 22-219, 22-218, 22-217, 22-216, 22-215,

21-221, 21-220, 21-219, 21-218, 21-217, 21-216, 21-215,

20-221, 20-220, 20-219, 20-218, 20-217, 20-216, 20-215,

19-221, 19-220, 19-219, 19-218, 19-217, 19-216, 19-215,

18-221, 18-220, 18-219, 18-218, 18-217, 18-216, 18-215,

17-221, 17-220, 17-219, 17-218, 17-217, 17-216, 17-215,

16-221, 16-220, 16-219, 16-218, 16-217, 16-216, 16-215,

### of SEQ ID NO:53.

Additional suitable fragments of murine PD-L2 include, but are not limited to, the following:

20-221, 33-222, 33-223, 33-224, 33-225, 33-226, 33-227,

21-221, 21-222, 21-223, 21-224, 21-225, 21-226, 21-227,

22-221, 22-222, 22-223, 22-224, 22-225, 22-226, 22-227,

23-221, 23-222, 23-223, 23-224, 23-225, 23-226, 23-227,

24-221, 24-222, 24-223, 24-224, 24-225, 24-226, 24-227,

of SEQ ID NO:1, optionally with one to five amino acids of a signal peptide attached to the N-terminal end. The signal peptide may be any disclosed herein, including the signal peptide contained within SEQ ID NO:1, or may be any signal peptide known in the art.

Exemplary suitable fragments of human PD-L2 that can be used as a first fusion partner include, but are not limited to, the following:

24-221, 24-220, 24-219, 24-218, 24-217, 24-216, 24-215,

23-221, 23-220, 23-219, 23-218, 23-217, 23-216, 23-215,

22-221, 22-220, 22-219, 22-218, 22-217, 22-216, 22-215,

21-221, 21-220, 21-219, 21-218, 21-217, 21-216, 21-215,

20-221, 20-220, 20-219, 20-218, 20-217, 20-216, 20-215,

19-221, 19-220, 19-219, 19-218, 19-217, 19-216, 19-215,

18-221, 18-220, 18-219, 18-218, 18-217, 18-216, 18-215,

17-221, 17-220, 17-219, 17-218, 17-217, 17-216, 17-215,

16-221, 16-220, 16-219, 16-218, 16-217, 16-216, 16-215,

### of SEQ ID NO:56.

Additional suitable fragments of human PD-L2 include, but are not limited to, the following:

20-221, 33-222, 33-223, 33-224, 33-225, 33-226, 33-227,

21-221, 21-222, 21-223, 21-224, 21-225, 21-226, 21-227,

22-221, 22-222, 22-223, 22-224, 22-225, 22-226, 22-227,

23-221, 23-222, 23-223, 23-224, 23-225, 23-226, 23-227,

24-221, 24-222, 24-223, 24-224, 24-225, 24-226, 24-227,

of SEQ ID NO:3, optionally with one to five amino acids of a signal peptide attached to the N-terminal end. The signal peptide may be any disclosed herein, including the signal peptide contained within SEQ ID NO:3, or may be any signal peptide known in the art.

Exemplary suitable fragments of non-human primate PD-L2 that can be used as a first fusion partner include, but are not limited to, the following:

24-221, 24-220, 24-219, 24-218, 24-217, 24-216, 24-215,

23-221, 23-220, 23-219, 23-218, 23-217, 23-216, 23-215,

22-221, 22-220, 22-219, 22-218, 22-217, 22-216, 22-215,

21-221, 21-220, 21-219, 21-218, 21-217, 21-216, 21-215,

20-221, 20-220, 20-219, 20-218, 20-217, 20-216, 20-215,

19-221, 19-220, 19-219, 19-218, 19-217, 19-216, 19-215,

18-221, 18-220, 18-219, 18-218, 18-217, 18-216, 18-215,

17-221, 17-220, 17-219, 17-218, 17-217, 17-216, 17-215,

16-221, 16-220, 16-219, 16-218, 16-217, 16-216, 16-215,

### of SEQ ID NO:5.

Additional suitable fragments of non-human primate PD-L2 include, but are not limited to, the following:

20-221, 33-222, 33-223, 33-224, 33-225, 33-226, 33-227,

21-221, 21-222, 21-223, 21-224, 21-225, 21-226, 21-227,

22-221, 22-222, 22-223, 22-224, 22-225, 22-226, 22-227,

23-221, 23-222, 23-223, 23-224, 23-225, 23-226, 23-227,

24-221, 24-222, 24-223, 24-224, 24-225, 24-226, 24-227,

of SEQ ID NO:5, optionally with one to five amino acids of a signal peptide attached to the N-terminal end. The signal peptide may be any disclosed herein, including the signal peptide contained within SEQ ID NO:5, or may be any signal peptide known in the art.

PD-L2 proteins also include a PD-1 binding fragment of amino acids 20-121 of SEQ ID NO:3 (human full length), or amino acids 1-102 of SEQ ID NO:23 (extracellular domain or ECD). In specific embodiments thereof, the PD-L2 polypeptide or PD-1 binding fragment also incorporates amino acids WDYKY at residues 110-114 of SEQ ID NO:3 or WDYKY at residues 91-95 of SEQ ID NO:23. By way of non-limiting examples, such a PD-1 binding fragment comprises at least 10, at least 20, at least 30, at least 40, at least 50, at least 60, at least 70, at least 75, at least 80, at least 85, at least 90, at least 95, or at least 100 contiguous amino acids of the sequence of amino acids 20-121 of SEQ ID NO:3, wherein a preferred embodiment of each such PD-1 binding fragment would comprise as a sub-fragment the amino acids WDYKY found at residues 110-114 of SEQ ID NO:3 or WDYKY at residues 91-95 of SEQ ID NO:23

2. PD-L1 Extracellular Domains

In one embodiment, the variant PD-L1 polypeptide includes all or part of the extracellular domain. The amino acid sequence of a representative extracellular domain of PD-L1 can have 80%, 85%, 90%, 95%, or 99% sequence identity to

| (SEQ ID NO: 32) | |
|---|---|
| FTVTVPKDLY VVEYGSNMTI ECKFPVEKQL DLAALIVYWE MEDKNIIQFV HGEEDLKVQH | 60 |
| SSYRQRARLL KDQLSLGNAA LQITDVKLQD AGVYRCMISY GGADYKRITV KVNAPYNKIN | 120 |
| QRILVVDPVT SEHELTCQAE GYPKAEVIWT SSDHQVLSGK TTTTNSKREE KLFNVTSTLR | 180 |
| INTTTNEIFY CTFRRLDPEE NHTAELVTPE LPLAHPPNER. | 220 |

The transmembrane domain of PD-L1 begins at amino acid position 239 of SEQ ID NO:9. It will be appreciated that the suitable fragments of PD-L1 can include 1, 2, 3, 4, 5, 6, 7, 8, 9, or 10 contiguous amino acids of a signal peptide sequence, for example SEQ ID NO:9 or variants thereof, 1, 2, 3, 4, 5, 6, 7, 8, 9, or 10 amino acids of the transmembrane domain, or combinations thereof.

The extracellular domain of murine PD-L1 has the following amino acid sequence

| (SEQ ID NO: 33) | |
|---|---|
| FTITAPKDLY VVEYGSNVTM ECRFPVEREL DLLALVVYWE KEDEQVIQFV AGEEDLKPQH | 60 |
| SNFRGRASLP KDQLLKGNAA LQITDVKLQD AGVYCCIISY GGADYKRITL KVNAPYRKIN | 120 |
| QRISVDPATS EHELICQAEG YPEAEVIWTN SDHQPVSGKR SVTTSRTEGM LLNVTSSLRV | 180 |
| NATANDVFYC TEWRSQPGQN HTAELIIPEL PATHPPQNRT HWVLLGSILL FLIVVSTVL. | 239 |

The transmembrane domain of the murine PD-L1 begins at amino acid position 240 of SEQ ID NO:7. In certain embodiments the PD-L1 polypeptide includes the extracellular domain of murine PD-L1 with 1, 2, 3, 4, 5, 6, 7, 8, 9, or 10 contiguous amino acids of a signal peptide, 1, 2, 3, 4, 5, 6, 7, 8, 9, or 10 contiguous amino acids of the transmembrane domain, or combinations thereof.

3. B7.1 Extracellular Domains

a. Murine B7.1 Extracellular Domains

In one embodiment, the PD-1 antagonist polypeptide includes the extracellular domain of murine B7.1 or a fragment thereof. The PD-1 antagonist polypeptide can be encoded by a nucleotide sequence having at least 80%, 85%, 90%, 95%, 99%, or 100% sequence identity to:

(SEQ ID NO: 34)

| | |
|---|---|
| atggcttgca attgtcagtt gatgcaggat acaccactcc tcaagtttcc atgtccaagg | 60 |

| | |
|---|---|
| ctcattcttc tctttgtgct gctgattcgt ctttcacaag tgtcttcaga tgttgatgaa | 120 |
| caactgtcca agtcagtgaa agataaggta ttgctgcctt gccgttacaa ctctcctcat | 180 |
| gaagatgagt ctgaagaccg aatctactgg caaaaacatg acaaagtggt gctgtctgtc | 240 |
| attgctggga aactaaaagt gtggcccgag tataagaacc ggactttata tgacaacact | 300 |
| acctactctc ttatcatcct gggcctggtc ctttcagacc ggggcacata cagctgtgtc | 360 |
| gttcaaaaga aggaaagagg aacgtatgaa gttaaacact tggctttagt aaagttgtcc | 420 |
| atcaaagctg acttctctac ccccaacata actgagtctg gaaacccatc tgcagacact | 480 |
| aaaaggatta cctgctttgc ttccgggggt ttcccaaagc ctcgcttctc ttggttggaa | 540 |
| aatggaagag aattacctgg catcaatacg acaatttccc aggatcctga atctgaattg | 600 |
| tacaccatta gtagccaact agatttcaat acgactcgca accacaccat taagtgtctc | 660 |
| attaaatatg gagatgctca cgtgtcagag gacttcacct gggaaaaacc cccagaagac | 720 |
| cctcctgata gcaagaac. | 738 |

In another embodiment, the PD-1 antagonist polypeptide can have at least 80%, 85%, 90%, 95%, 99%, or 100% sequence identity to the murine amino acid sequence:

| (SEQ ID NO: 35) | |
|---|---|
| MACNCQLMQD TPLLKFPCPR LILLFVLLIR LSQVSSDVDE QLSKSVKDKV LLPCRYNSPH | 60 |
| EDESEDRIYW QKHDKVVLSV IAGKLKVWPE YKNRTLYDNT TYSLIILGLV LSDRGTYSCV | 120 |
| VQKKERGTYE VKHLALVKLS IKADFSTPNI TESGNPSADT KRITCFASGG FPKPRFSWLE | 180 |
| NGRELPGINT TISQDPESEL YTISSQLDFN TTRNHTIKCL IKYGDAHVSE DFTWEKPPED | 240 |
| PPDSKN. | 246 |

The signal sequence will be removed in the mature protein. Additionally, signal peptides from other organisms can be used to enhance the secretion of the protein from a host during manufacture. SEQ ID NO:36 provides the murine amino acid sequence of SEQ ID NO:35 without the signal sequence:

(SEQ ID NO: 36)

| | |
|---|---|
| VDEQLSKSVK DKVLLPCRYN SPHEDESEDR IYWQKHDKVV LSVIAGKLKV WPEYKNRTLY | 60 |

| | |
|---|---|
| DNTTYSLIIL GLVLSDRGTY SCVVQKKERG TYEVKHLALV KLSIKADFST PNITESGNPS | 120 |
| ADTKRITCFA SGGFPKPRFS WLENGRELPG INTTISQDPE SELYTISSQL DFNTTRNHTI | 180 |
| KCLIKYGDAH VSEDFTWEKP PEDPPDSKN. | 209 |

In another embodiment, the PD-1 antagonist polypeptide includes the IgV domain of murine B7.1. The first fusion partner can be encoded by a nucleotide sequence having at least 80%, 85%, 90%, 95%, 99%, or 100% sequence identity to:

| (SEQ ID NO: 37) | |
|---|---|
| gttgatgaac aactgtccaa gtcagtgaaa gataaggtat tgctgccttg ccgttacaac | 60 |
| tctcctcatg aagatgagtc tgaagaccga atctactggc aaaaacatga caaagtggtg | 120 |
| ctgtctgtca ttgctgggaa actaaaagtg tggcccgagt ataagaaccg gactttatat | 180 |
| gacaacacta cctactctct tatcatcctg ggcctggtcc tttcagaccg gggcacatac | 240 |
| agctgtgtcg ttcaaaagaa ggaaagagga acgtatgaag ttaaacactt g. | 291 |

The PD-1 antagonist polypeptide can have at least 80%, 85%, 90%, 95%, 99%, or 100% sequence identity to the murine amino acid sequence:

| (SEQ ID NO: 38) | |
|---|---|
| VDEQLSKSVK DKVLLPCRYN SPHEDESEDR IYWQKHDKVV LSVIAGKLKV WPEYKNRTLY | 60 |
| DNTTYSLIIL GLVLSDRGTY SCVVQKKERG TYEVKHL, | 97 |

also referred to as B7.1V.

b. Human B7.1 Extracellular Domains

In one embodiment, the PD-1 antagonist polypeptide includes the extracellular domain of human B7.1 or a fragment thereof. The PD-1 antagonist polypeptide can be encoded by a nucleotide sequence having at least 80%, 85%, 90%, 95%, 99%, or 100% sequence identity to:

| (SEQ ID NO: 39) | |
|---|---|
| atgggccaca cacggaggca gggaacatca ccatccaagt gtccatacct caatttcttt | 60 |
| cagctcttgg tgctggctgg tctttctcac ttctgttcag gtgttatcca cgtgaccaag | 120 |
| gaagtgaaag aagtggcaac gctgtcctgt ggtcacaatg tttctgttga agagctggca | 180 |
| caaactcgca tctactggca aaaggagaag aaaatggtgc tgactatgat gtctggggac | 240 |
| atgaatatat ggcccgagta caagaaccgg accatctttg atatcactaa taacctctcc | 300 |
| attgtgatcc tggctctgcg cccatctgac gagggcacat acgagtgtgt tgttctgaag | 360 |
| tatgaaaaag acgctttcaa gcgggaacac ctggctgaag tgacgttatc agtcaaagct | 420 |
| gacttcccta cacctagtat atctgacttt gaaattccaa cttctaatat tagaaggata | 480 |
| atttgctcaa cctctggagg ttttccagag cctcacctct cctggttgga aaatggagaa | 540 |
| gaattaaatg ccatcaacac aacagtttcc caagatcctg aaactgagct ctatgctgtt | 600 |
| agcagcaaac tggatttcaa tatgacaacc aaccacagct tcatgtgtct catcaagtat | 660 |
| ggacatttaa gagtgaatca gaccttcaac tggaatacaa ccaagcaaga gcattttcct | 720 |
| gataacctgc tc. | 732 |

In another embodiment, the PD-1 antagonist polypeptide can have at least 80%, 85%, 90%, 95%, 99%, or 100% sequence identity to the human amino acid sequence:

| (SEQ ID NO: 40) | |
|---|---|
| MIFLLLMLSL ELQLHQIAAL FTVTVPKELYIIEHGSNVTL | |
| MGHTRRQGTS PSKGPYLNFF QLLVLAGLSH FCSGVIHVTK EVKEVATLSC GHNVSVEELA | 60 |
| QTRIYWQKEK KMVLTMMSGD MNIWPEYKNR TIFDITNNLS IVILALRPSD EGTYECVVLK | 120 |
| YEKDAFKREH LAEVTLSVKA DFPTPSISDF EIPTSNIRRI ICSTSGGFPE PHLSWLENGE | 180 |
| ELNAINTTVS QDPETELYAV SSKLDFNMTT NHSFMCLIKY GHLRVNQTFN WNTTKQEHFP | 240 |
| DNL. | 243 |

The signal sequence will be removed in the mature protein. Additionally, signal peptides from other organisms can be used to enhance the secretion of the protein from a host during manufacture. SEQ ID NO:41 provides the human amino acid sequence of SEQ ID NO:40 without the signal sequence:

| (SEQ ID NO: 41) | |
|---|---|
| VIHVTKEVKE VATLSCGHNV SVEELAQTRI YWQKEKKMVL TMMSGDMNIW PEYKNRTIFD | 60 |
| ITNNLSIVIL ALRPSDEGTY ECVVLKYEKD AFKREHLAEV TLSVKADFPT PSISDFEIPT | 120 |
| SNIRRIICST SGGFPEPHLS WLENGEELNA INTTVSQDPE TELYAVSSKL DFNMTTNHSF | 180 |
| MCLIKYGHLR VNQTFNWNTT KQEHFPDNL. | 209 |

In another embodiment, the PD-1 antagonist polypeptide includes the IgV domain of human 87.1. The first fusion partner can be encoded by a nucleotide sequence having at least 80%, 85%, 90%, 95%, 99%, or 100% sequence identity to:

| (SEQ ID NO: 42) | |
|---|---|
| gttatccacg tgaccaagga agtgaaagaa gtggcaacgc tgtcctgtgg tcacaatgtt | 60 |
| tctgttgaag agctggcaca aactcgcatc tactggcaaa aggagaagaa aatggtgctg | 120 |
| actatgatgt ctggggacat gaatatatgg cccgagtaca agaaccggac catctttgat | 180 |
| atcactaata acctctccat tgtgatcctg gctctgcgcc catctgacga gggcacatac | 240 |
| gagtgtgttg ttctgaagta tgaaaaagac gctttcaagc gggaacacct ggctgaagtg | 300 |
| acg. | 303 |

The PD-1 antagonist polypeptide can have at least 80%, 85%, 90%, 95%, 99%, or 100% sequence identity to the human amino acid sequence:

| (SEQ ID NO: 43) | |
|---|---|
| VIHVTKEVKE VATLSCGHNV SVEELAQTRI YWQKEKKMVL TMMSGDMNIW PEYKNRTIFD | 60 |
| ITNNLSIVIL ALRPSDEGTY ECVVLKYEKD AFKREHLAEV T, | 101 |
| also referred to as B7.1V. | |

3. B7.1 Extracellular Domain Fragments

Exemplary suitable fragments of murine B7.1 that can be used as a costimulatory polypeptide domain include, but are not limited to, the following:

42-246, 42-245, 42-244, 42-243, 42-242, 42-241, 42-240,

41-246, 41-245, 41-244, 41-243, 41-242, 41-241, 41-240,

40-246, 40-245, 40-244, 40-243, 40-242, 40-241, 40-240,

39-246, 39-245, 39-244, 39-243, 39-242, 39-241, 39-240,

38-246, 38-245, 38-244, 38-243, 38-242, 38-241, 38-240,

37-246, 37-245, 37-244, 37-243, 37-242, 37-241, 37-240,

36-246, 36-245, 36-244, 36-243, 36-242, 36-241, 36-240,

35-246, 35-245, 35-244, 35-243, 35-242, 35-241, 35-240,

34-246, 34-245, 34-244, 34-243, 34-242, 34-241, 34-240,

### of SEQ ID NO:11.

Additional suitable fragments of murine B7.1 include, but are not limited to, the following:

38-246, 38-247, 38-248, 38-249, 38-250, 38-251, 38-252,

39-246, 39-247, 39-248, 39-249, 39-250, 39-251, 39-252,

40-246, 40-247, 40-248, 40-249, 40-250, 40-251, 40-252,

41-246, 41-247, 41-248, 41-249, 41-250, 41-251, 41-252,

42-246, 42-247, 42-248, 42-249, 42-250, 42-251, 42-252,

of SEQ ID NO:11, optionally with one to five amino acids of a signal peptide attached to the N-terminal end. The signal peptide may be any disclosed herein, including the signal peptide contained within SEQ ID NO:11, or may be any signal peptide known in the art.

Exemplary suitable fragments of human B7.1 that can be used as a costimulatory polypeptide domain include, but are not limited to, the following:

39-243, 39-242, 39-241, 39-240, 39-239, 39-238, 39-237,

38-243, 38-242, 38-241, 38-240, 38-239, 38-238, 38-237,

37-243, 37-242, 37-241, 37-240, 37-239, 37-238, 37-237,

36-243, 36-242, 36-241, 36-240, 36-239, 36-238, 36-237,

35-243, 35-242, 35-241, 35-190, 35-239, 35-238, 35-237,

34-243, 34-242, 34-241, 34-240, 34-239, 34-238, 34-237,

33-243, 33-242, 33-241, 33-240, 33-239, 33-238, 33-237,

32-243, 32-242, 32-241, 32-240, 32-239, 32-238, 32-237,

31-243, 31-242, 31-241, 31-240, 31-239, 31-238, 31-237,

### of SEQ ID NO:13.

Additional suitable fragments of human B7.1 include, but are not limited to, the following:

35-243, 35-244, 35-245, 35-246, 35-247, 35-248, 35-249,

36-243, 36-244, 36-245, 36-246, 36-247, 36-248, 36-249,

37-243, 37-244, 37-245, 37-246, 37-247, 37-248, 37-249,

38-243, 38-244, 38-245, 38-246, 38-247, 38-248, 38-249,

39-243, 39-244, 39-245, 39-246, 39-247, 39-248, 39-249,

of SEQ ID NO:13, optionally with one to five amino acids of a signal peptide attached to the N-terminal end. The signal peptide may be any disclosed herein, including the signal peptide contained within SEQ ID NO:13, or may be any signal peptide known in the art.

E. Variants

1. Variant PD-L2 and PD-L1 PD-1 Antagonists

Additional PD-1 antagonists include PD-L2 and PD-L1, polypeptides and fragments thereof that are mutated so that they retain the ability to bind to PD-1 under physiological conditions, have increased binding to PD-1, or have decreased binding to PD-1 compared to non-mutated PD-1 but are not able to promote signal transduction through the PD-1 receptor. One embodiment provides isolated PD-L2 and PD-L1 polypeptides that contain one or more amino acid substitutions, deletions, or insertions that inhibit or reduce the ability of the polypeptide to activate PD-1 and transmit an inhibitory signal to a T cell compared to non-mutated PD-L2 or PD-L1. The PD-L2 and PD-L1 polypeptides may be of any species of origin. In one embodiment, the PD-L2 or PD-L1 polypeptide is from a mammalian species. In a preferred embodiment, the PD-L2 or PD-L1 polypeptide is of human or non-human primate origin.

In another embodiment the variant PD-L2 or PD-L1 polypeptide has the same binding activity to PD-1 as wildtype or non-variant PD-L2 or PD-L1 but does not have or has less than 10% ability to stimulate signal transduction through the PD-1 receptor relative to a non-mutated PD-L2 or PD-L1 polypeptide. In other embodiments, the variant PD-L2 or PD-L1 polypeptide has 10%, 20%, 30%, 40%, 50%, 60%, 70%, 80%, 90%, 100% or more binding activity to PD-1 than wildtype PD-L2 or PD-L1 and has less than 50%, 40%, 30%, 20%, or 10% of the ability to stimulate signal transduction through the PD-1 receptor relative to a non-mutated PD-L2 or PD-L1 polypeptide.

A variant PD-L2 or PD-L1 polypeptide can have any combination of amino acid substitutions, deletions or insertions. In one embodiment, isolated PD-L2 or PD-L1 variant polypeptides have an integer number of amino acid alterations such that their amino acid sequence shares at least 60, 70, 80, 85, 90, 95, 97, 98, 99, 99.5 or 100% identity with an amino acid sequence of a wild type PD-L2 or PD-L1 polypeptide. In a preferred embodiment, B7-H1 variant polypeptides have an amino acid sequence sharing at least 60, 70, 80, 85, 90, 95, 97, 98, 99, 99.5 or 100% identity with the amino acid sequence of a wild type murine, non-human primate or human PD-L2 or PD-L1 polypeptide.

Percent sequence identity can be calculated using computer programs or direct sequence comparison. Preferred computer program methods to determine identity between two sequences include, but are not limited to, the GCG program package, FASTA, BLASTP, and TBLASTN (see, e.g., D. W. Mount, 2001, Bioinformatics: Sequence and Genome Analysis, Cold Spring Harbor Laboratory Press, Cold Spring Harbor, N.Y.). The BLASTP and TBLASTN programs are publicly available from NCBI and other sources. The well-known Smith Waterman algorithm may also be used to determine identity.

Exemplary parameters for amino acid sequence comparison include the following: 1) algorithm from Needleman and Wunsch (J. Mol. Biol., 48:443-453 (1970)); 2) BLOSSUM62 comparison matrix from Hentikoff and Hentikoff (Proc. Natl. Acad. Sci. U.S.A., 89:10915-10919 (1992)) 3) gap penalty=12; and 4) gap length penalty=4. A program useful with these parameters is publicly available as the “gap” program (Genetics Computer Group, Madison, Wis.). The aforementioned parameters are the default parameters for polypeptide comparisons (with no penalty for end gaps).

Alternatively, polypeptide sequence identity can be calculated using the following equation: % identity=(the number of identical residues)/(alignment length in amino acid residues)*100. For this calculation, alignment length includes internal gaps but does not include terminal gaps.

Amino acid substitutions in PD-L2 or PD-L1 polypeptides may be “conservative” or “non-conservative”. As used herein, “conservative” amino acid substitutions are substitutions wherein the substituted amino acid has similar structural or chemical properties, and “non-conservative” amino acid substitutions are those in which the charge, hydrophobicity, or bulk of the substituted amino acid is significantly altered. Non-conservative substitutions will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain.

Examples of conservative amino acid substitutions include those in which the substitution is within one of the five following groups: 1) small aliphatic, nonpolar or slightly polar residues (Ala, Ser, Thr, Pro, Gly); 2) polar, negatively charged residues and their amides (Asp, Asn, Glu, Gln); polar, positively charged residues (His, Arg, Lys); large aliphatic, nonpolar residues (Met, Leu, Ile, Val, Cys); and large aromatic resides (Phe, Tyr, Trp). Examples of non-conservative amino acid substitutions are those where 1) a hydrophilic residue, e.g., seryl or threonyl, is substituted for (or by) a hydrophobic residue, e.g., leucyl, isoleucyl, phenylalanyl, valyl, or alanyl; 2) a cysteine or proline is substituted for (or by) any other residue; 3) a residue having an electropositive side chain, e.g., lysyl, arginyl, or histidyl, is substituted for (or by) an electronegative residue, e.g., glutamyl or aspartyl; or 4) a residue having a bulky side chain, e.g., phenylalanine, is substituted for (or by) a residue that does not have a side chain, e.g., glycine.

It is understood, however, that substitutions at the recited amino acid positions can be made using any amino acid or amino acid analog. For example, the substitutions at the recited positions can be made with any of the naturally-occurring amino acids (e.g., alanine, aspartic acid, asparagine, arginine, cysteine, glycine, glutamic acid, glutamine, histidine, leucine, valine, isoleucine, lysine, methionine, proline, threonine, serine, phenylalanine, tryptophan, or tyrosine).

While the substitutions described herein are with respect to mouse, non-human primate and human PD-L2 or PD-L1, it is noted that one of ordinary skill in the art could readily make equivalent alterations in the corresponding polypeptides from other species (e.g., rat, hamster, guinea pig, gerbil, rabbit, dog, cat, horse, pig, sheep or cow). However, since binding has a species-specific component, it is preferable to use human when administering PD-1 antagonists to humans.

In one embodiment, the disclosed isolated variant PD-L2 or PD-L1 polypeptides are antagonists of PD-1 and bind to and block PD-1 without triggering signal transduction through PD-1. By preventing the attenuation of T cells by PD-1 signal transduction, more T cells are available to be activated. Preventing T cell inhibition enhances T cell responses, enhances proliferation of T cells, enhances production and/or secretion of cytokines by T cells, stimulates differentiation and effector functions of T cells or promotes survival of T cells relative to T cells not contacted with a PD-1 antagonist. The T cell response that results from the interaction typically is greater than the response in the absence of the PD-1 antagonist polypeptide. The response of the T cell in the absence of the PD-1 antagonist polypeptide can be no response or can be a response significantly lower than in the presence of the PD-1 antagonist polypeptide. The response of the T cell can be an effector (e.g., CTL or antibody-producing B cell) response, a helper response providing help for one or more effector (e.g., CTL or antibody-producing B cell) responses, or a suppressive response.

Methods for measuring the binding affinity between two molecules are well known in the art. Methods for measuring the binding affinity of variant PD-L2 or PD-L1 polypeptides for PD-1 include, but are not limited to, fluorescence activated cell sorting (FACS), surface plasmon resonance, fluorescence anisotropy, affinity chromatography and affinity selection-mass spectrometry.

The variant polypeptides disclosed herein can be full-length polypeptides, or can be a fragment of a full length polypeptide. Preferred fragments include all or part of the extracellular domain of effective to bind to PD-1. As used herein, a fragment refers to any subset of the polypeptide that is a shorter polypeptide of the full length protein.

2. Variant B7.1 and PD-1 Antagonists

Additional PD-1 antagonists include B7.1 and PD-1 polypeptides and fragments thereof that are modified so that they retain the ability to bind to PD-L2 and/or PD-L1 under physiological conditions, have increased binding, or have decreased binding to PD-L2 and/or PD-L1. The B7.1 and PD-1 polypeptides may be of any species of origin. In one embodiment, the B7.1 or PD-1 polypeptide is from a mammalian species. In a preferred embodiment, the B7.1 or PD-1 polypeptide is of human or non-human primate origin.

A variant B7.1 or PD-1 polypeptide can have any combination of amino acid substitutions, deletions or insertions. In one embodiment, isolated B7.1 or PD-1 variant polypeptides have an integer number of amino acid alterations such that their amino acid sequence shares at least 60, 70, 80, 85, 90, 95, 97, 98, 99, 99.5 or 100% identity with an amino acid sequence of a wild type B7.1 or PD-1 polypeptide. In a preferred embodiment, B7.1 or PD-1 variant polypeptides have an amino acid sequence sharing at least 60, 70, 80, 85, 90, 95, 97, 98, 99, 99.5 or 100% identity with the amino acid sequence of a wild type murine, non-human primate or human B7.1 or PD-1 polypeptide.

Amino acid substitutions in B7.1 or PD-1 polypeptides may be “conservative” or “non-conservative”. Conservative and non-conservative substitutions are described above.

In one embodiment, the disclosed isolated variant B7.1 or PD-1 polypeptides are antagonists of PD-1 and bind to PD-L2 and/or PD-L1, thereby blocking their binding to endogenous PD-1. By preventing the attenuation of T cells by PD-1 signal transduction, more T cells are available to be activated. Preventing T cell inhibition enhances T cell responses, enhances proliferation of T cells, enhances production and/or secretion of cytokines by T cells, stimulates differentiation and effector functions of T cells or promotes survival of T cells relative to T cells not contacted with a PD-1 antagonist. The T cell response that results from the interaction typically is greater than the response in the absence of the PD-1 antagonist polypeptide. The response of the T cell in the absence of the PD-1 antagonist polypeptide can be no response or can be a response significantly lower than in the presence of the PD-1 antagonist polypeptide. The response of the T cell can be an effector (e.g., CTL or antibody-producing B cell) response, a helper response providing help for one or more effector (e.g., CTL or antibody-producing B cell) responses, or a suppressive response.

The variant polypeptides can be full-length polypeptides, or can be a fragment of a full length polypeptide. Preferred fragments include all or part of the extracellular domain of effective to bind to PD-L2 and/or PD-L1. As used herein, a fragment refers to any subset of the polypeptide that is a shorter polypeptide of the full length protein.

F. Fusion Proteins

In some embodiments, the PD-1 antagonists are fusion proteins that contain a first polypeptide domain and a second targeting domain that is an antigen-binding domain that targets the fusion protein to tumor cells or tumor cell-associated neovasculature. The fusion protein can either bind to a T cell receptor and enhance a T cell response or preferably the fusion protein can bind to and block inhibitory signal transduction into the T cell, for example by competitively binding to PD-1. By interfering with natural inhibitory ligands binding PD-1, the disclosed compositions effectively block signal transduction through PD-1. Suitable costimulatory polypeptides include variant polypeptides and/or fragments thereof that have increased or decreased binding affinity to inhibitory T cell signal transduction receptors such as PD-1.

The fusion proteins also optionally contain a peptide or polypeptide linker domain that separates the first polypeptide domain from the antigen-binding domain.

Fusion proteins disclosed herein are of formula I:

N—R₁—R₂—R₃—C

wherein “N” represents the N-terminus of the fusion protein, “C” represents the C-terminus of the fusion protein, “R₁” is a PD-L2, PD-L1, B7.1, or PD-1 polypeptide or a antigen-binding targeting domain, “R₂” is a peptide/polypeptide linker domain, and “R₃” is a targeting domain or a antigen-binding targeting domain, wherein “R₃” is a polypeptide domain when “R₁” is a antigen-binding targeting domain, and “R₃” is a antigen-binding targeting domain when “R₁” is a PD-L2, PD-L1, B7.1, or PD-1 polypeptide domain. In a preferred embodiment, “R₁” is a PD-L2, PD-L1, B7.1, or PD-1 polypeptide domain and “R₃” is a antigen-binding targeting domain.

Optionally, the fusion proteins additionally contain a domain that functions to dimerize or multimerize two or more fusion proteins. The domain that functions to dimerize or multimerize the fusion proteins can either be a separate domain, or alternatively can be contained within one of one of the other domains (PD-L2, PD-L1, B7.1, or PD-1 polypeptide domain, antigen-binding targeting domain, or peptide/polypeptide linker domain) of the fusion protein.

The fusion proteins can be dimerized or multimerized. Dimerization or multimerization can occur between or among two or more fusion proteins through dimerization or multimerization domains. Alternatively, dimerization or multimerization of fusion proteins can occur by chemical crosslinking. The dimers or multimers that are formed can be homodimeric/homomultimeric or heterodimeric/heteromultimeric.

The modular nature of the fusion proteins and their ability to dimerize or multimerize in different combinations provides a wealth of options for targeting molecules that function to enhance an immune response to the tumor cell microenvironment.

1. Antigen-Binding Targeting Domain

The fusion proteins also contain antigen-binding targeting domains. In some embodiments, the targeting domains bind to antigens, ligands or receptors that are specific to tumor cells or tumor-associated neovasculature, or are upregulated in tumor cells or tumor-associated neovasculature compared to normal tissue. In some embodiments, the targeting domains bind to antigens, ligands or receptors that are specific to immune tissue involved in the regulation of T cell activation in response to infectious disease causing agents.

Tumor/Tumor-Associated Vasculature Targeting Domains

Antigens, Ligands and Receptors to Target

Tumor-Specific and Tumor-Associated Antigens

In one embodiment the fusion proteins contain a domain that specifically binds to an antigen that is expressed by tumor cells. The antigen expressed by the tumor may be specific to the tumor, or may be expressed at a higher level on the tumor cells as compared to non-tumor cells. Antigenic markers such as serologically defined markers known as tumor associated antigens, which are either uniquely expressed by cancer cells or are present at markedly higher levels (e.g., elevated in a statistically significant manner) in subjects having a malignant condition relative to appropriate controls, are contemplated for use in certain embodiments.

Tumor-associated antigens may include, for example, cellular oncogene-encoded products or aberrantly expressed proto-oncogene-encoded products (e.g., products encoded by the neu, ras, trk, and kit genes), or mutated forms of growth factor receptor or receptor-like cell surface molecules (e.g., surface receptor encoded by the c-erbB gene). Other tumor-associated antigens include molecules that may be directly involved in transformation events, or molecules that may not be directly involved in oncogenic transformation events but are expressed by tumor cells (e.g., carcinoembryonic antigen, CA-125, melonoma associated antigens, etc.) (see, e.g., U.S. Pat. No. 6,699,475; Jager, et al., Int. J. Cancer, 106:817-20 (2003); Kennedy, et al., Int. Rev. Immunol., 22:141-72 (2003); Scanlan, et al. Cancer Immun., 4:1 (2004)).

Genes that encode cellular tumor associated antigens include cellular oncogenes and proto-oncogenes that are aberrantly expressed. In general, cellular oncogenes encode products that are directly relevant to the transformation of the cell, and because of this, these antigens are particularly preferred targets for immunotherapy. An example is the tumorigenic neu gene that encodes a cell surface molecule involved in oncogenic transformation. Other examples include the ras, kit, and trk genes. The products of proto-oncogenes (the normal genes which are mutated to form oncogenes) may be aberrantly expressed (e.g., overexpressed), and this aberrant expression can be related to cellular transformation. Thus, the product encoded by proto-oncogenes can be targeted. Some oncogenes encode growth factor receptor molecules or growth factor receptor-like molecules that are expressed on the tumor cell surface. An example is the cell surface receptor encoded by the c-erbB gene. Other tumor-associated antigens may or may not be directly involved in malignant transformation. These antigens, however, are expressed by certain tumor cells and may therefore provide effective targets. Some examples are carcinoembryonic antigen (CEA), CA 125 (associated with ovarian carcinoma), and melanoma specific antigens.

In ovarian and other carcinomas, for example, tumor associated antigens are detectable in samples of readily obtained biological fluids such as serum or mucosal secretions. One such marker is CA125, a carcinoma associated antigen that is also shed into the bloodstream, where it is detectable in serum (e.g., Bast, et al., N. Eng. J. Med., 309:883 (1983); Lloyd, et al., Int. J. Canc., 71:842 (1997). CA125 levels in serum and other biological fluids have been measured along with levels of other markers, for example, carcinoembryonic antigen (CEA), squamous cell carcinoma antigen (SCC), tissue polypeptide specific antigen (TPS), sialyl TN mucin (STN), and placental alkaline phosphatase (PLAP), in efforts to provide diagnostic and/or prognostic profiles of ovarian and other carcinomas (e.g., Sarandakou, et al., Acta Oncol., 36:755 (1997); Sarandakou, et al., Eur. J. Gynaecol. Oncol., 19:73 (1998); Meier, et al., Anticancer Res., 17(4B):2945 (1997); Kudoh, et al., Gynecol. Obstet. Invest., 47:52 (1999)). Elevated serum CA125 may also accompany neuroblastoma (e.g., Hirokawa, et al., Surg. Today, 28:349 (1998), while elevated CEA and SCC, among others, may accompany colorectal cancer (Gebauer, et al., Anticancer Res., 17(4B):2939 (1997)).

The tumor associated antigen, mesothelin, defined by reactivity with monoclonal antibody K-1, is present on a majority of squamous cell carcinomas including epithelial ovarian, cervical, and esophageal tumors, and on mesotheliomas (Chang, et al., Cancer Res., 52:181 (1992); Chang, et al., Int. J. Cancer, 50:373 (1992); Chang, et al., Int. J. Cancer, 51:548 (1992); Chang, et al., Proc. Natl. Acad. Sci. USA, 93:136 (1996); Chowdhury, et al., Proc. Natl. Acad. Sci. USA, 95:669 (1998)). Using MAb K-1, mesothelin is detectable only as a cell-associated tumor marker and has not been found in soluble form in serum from ovarian cancer patients, or in medium conditioned by OVCAR-3 cells (Chang, et al., Int. J. Cancer, 50:373 (1992)). Structurally related human mesothelin polypeptides, however, also include tumor-associated antigen polypeptides such as the distinct mesothelin related antigen (MRA) polypeptide, which is detectable as a naturally occurring soluble antigen in biological fluids from patients having malignancies (see WO 00/50900).

A tumor antigen may include a cell surface molecule. Tumor antigens of known structure and having a known or described function, include the following cell surface receptors: HER1 (GenBank Accession No. U48722), HER2 (Yoshino, et al., J. Immunol., 152:2393 (1994); Disis, et al., Canc. Res., 54:16 (1994); GenBank Acc. Nos. X03363 and M17730), HER3 (GenBank Acc. Nos. U29339 and M34309), HER4 (Plowman, et al., Nature, 366:473 (1993); GenBank Acc. Nos. L07868 and T64105), epidermal growth factor receptor (EGFR) (GenBank Acc. Nos. U48722, and KO3193), vascular endothelial cell growth factor (GenBank No. M32977), vascular endothelial cell growth factor receptor (GenBank Acc. Nos. AF022375, 1680143, U48801 and X62568), insulin-like growth factor-I (GenBank Acc. Nos. X00173, X56774, X56773, X06043, European Patent No. GB 2241703), insulin-like growth factor-II (GenBank Acc. Nos. X03562, X00910, M17863 and M17862), transferrin receptor (Trowbridge and Omary, Proc. Nat. Acad. USA, 78:3039 (1981); GenBank Acc. Nos. X01060 and M11507), estrogen receptor (GenBank Acc. Nos. M38651, X03635, X99101, U47678 and M12674), progesterone receptor (GenBank Acc. Nos. X51730, X69068 and M15716), follicle stimulating hormone receptor (FSH-R) (GenBank Acc. Nos. Z34260 and M65085), retinoic acid receptor (GenBank Acc. Nos. L12060, M60909, X77664, X57280, X07282 and X06538), MUC-1 (Barnes, et al., Proc. Nat. Acad. Sci. USA, 86:7159 (1989); GenBank Acc. Nos. M65132 and M64928) NY-ESO-1 (GenBank Acc. Nos. AJ003149 and U87459), NA 17-A (PCT Publication No. WO 96/40039), Melan-A/MART-1 (Kawakami, et al., Proc. Nat. Acad. Sci. USA, 91:3515 (1994); GenBank Acc. Nos. U06654 and U06452), tyrosinase (Topalian, et al., Proc. Nat. Acad. Sci. USA, 91:9461 (1994); GenBank Acc. No. M26729; Weber, et al., J. Clin. Invest, 102:1258 (1998)), Gp-100 (Kawakami, et al., Proc. Nat. Acad. Sci. USA, 91:3515 (1994); GenBank Acc. No. S73003, Adema, et al., J. Biol. Chem., 269:20126 (1994)), MAGE (van den Bruggen, et al., Science, 254:1643 (1991)); GenBank Acc. Nos. U93163, AF064589, U66083, D32077, D32076, D32075, U10694, U10693, U10691, U10690, U10689, U10688, U10687, U10686, U10685, L18877, U10340, U10339, L18920, U03735 and M77481), BAGE (GenBank Acc. No. U19180; U.S. Pat. Nos. 5,683,886 and 5,571,711), GAGE (GenBank Acc. Nos. AF055475, AF055474, AF055473, U19147, U19146, U19145, U19144, U19143 and U19142), any of the CTA class of receptors including in particular HOM-MEL-40 antigen encoded by the SSX2 gene (GenBank Acc. Nos. X86175, U90842, U90841 and X86174), carcinoembryonic antigen (CEA, Gold and Freedman, J. Exp. Med., 121:439 (1985); GenBank Acc. Nos. M59710, M59255 and M29540), and PyLT (GenBank Acc. Nos. J02289 and J02038); p97 (melanotransferrin) (Brown, et al., J. Immunol., 127:539-46 (1981); Rose, et al., Proc. Natl. Acad. Sci. USA, 83:1261-61 (1986)).

Additional tumor associated antigens include prostate surface antigen (PSA) (U.S. Pat. Nos. 6,677,157; 6,673,545); β-human chorionic gonadotropin β-HCG) (McManus, et al., Cancer Res., 36:3476-81 (1976); Yoshimura, et al., Cancer, 73:2745-52 (1994); Yamaguchi, et al., Br. J. Cancer, 60:382-84 (1989): Alfthan, et al., Cancer Res., 52:4628-33 (1992)); glycosyltransferase β-1,4-N-acetylgalactosaminyltransferases (GalNAc) (Hoon, et al., Int. J. Cancer, 43:857-62 (1989); Ando, et al., Int. J. Cancer, 40:12-17 (1987); Tsuchida, et al., J. Natl. Cancer, 78:45-54 (1987); Tsuchida, et al., J. Natl. Cancer, 78:55-60 (1987)); NUC18 (Lehmann, et al., Proc. Natl. Acad. Sci. USA, 86:9891-95 (1989); Lehmann, et al., Cancer Res., 47:841-45 (1987)); melanoma antigen gp75 (Vijayasardahi, et al., J. Exp. Med., 171:1375-80 (1990); GenBank Accession No. X51455); human cytokeratin 8; high molecular weight melanoma antigen (Natali, et al., Cancer, 59:55-63 (1987); keratin 19 (Datta, et al., J. Clin. Oncol., 12:475-82 (1994)).

Tumor antigens of interest include antigens regarded in the art as “cancer/testis” (CT) antigens that are immunogenic in subjects having a malignant condition (Scanlan, et al., Cancer Immun., 4:1 (2004)). CT antigens include at least 19 different families of antigens that contain one or more members and that are capable of inducing an immune response, including but not limited to MAGEA (CT1); BAGE (CT2); MAGEB (CT3); GAGE (CT4); SSX (CT5); NY-ESO-1 (CT6); MAGEC (CT7); SYCP1 (C8); SPANXB1 (CT11.2); NA88 (CT18); CTAGE (CT21); SPA17 (CT22); OY-TES-1 (CT23); CAGE (CT26); HOM-TES-85 (CT28); HCA661 (CT30); NY-SAR-35 (CT38); FATE (CT43); and TPTE (CT44).

Additional tumor antigens that can be targeted, including a tumor-associated or tumor-specific antigen, include, but not limited to, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pml-RARα fusion protein, PTPRK, K-ras, N-ras, Triosephosphate isomeras, Bage-1, Gage 3, 4, 5, 6, 7, GnTV, Herv-K-mel, Lage-1, Mage-A1, 2, 3, 4, 6, 10, 12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGS), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, α-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, and TPS. Other tumor-associated and tumor-specific antigens are known to those of skill in the art and are suitable for targeting by the disclosed fusion proteins.

Antigens Associated with Tumor Neovasculature

Protein therapeutics can be ineffective in treating tumors because they are inefficient at tumor penetration. Tumor-associated neovasculature provides a readily accessible route through which protein therapeutics can access the tumor. In another embodiment the fusion proteins contain a domain that specifically binds to an antigen that is expressed by neovasculature associated with a tumor.

The antigen may be specific to tumor neovasculature or may be expressed at a higher level in tumor neovasculature when compared to normal vasculature. Exemplary antigens that are over-expressed by tumor-associated neovasculature as compared to normal vasculature include, but are not limited to, VEGF/KDR, Tie2, vascular cell adhesion molecule (VCAM), endoglin and α₅β₃ integrin/vitronectin. Other antigens that are over-expressed by tumor-associated neovasculature as compared to normal vasculature are known to those of skill in the art and are suitable for targeting by the disclosed fusion proteins.

Chemokines/Chemokine Receptors

In another embodiment, the fusion proteins contain a domain that specifically binds to a chemokine or a chemokine receptor. Chemokines are soluble, small molecular weight (8-14 kDa) proteins that bind to their cognate G-protein coupled receptors (GPCRs) to elicit a cellular response, usually directional migration or chemotaxis. Tumor cells secrete and respond to chemokines, which facilitate growth that is achieved by increased endothelial cell recruitment and angiogenesis, subversion of immunological surveillance and maneuvering of the tumoral leukocyte profile to skew it such that the chemokine release enables the tumor growth and metastasis to distant sites. Thus, chemokines are vital for tumor progression.

Based on the positioning of the conserved two N-terminal cysteine residues of the chemokines, they are classified into four groups namely CXC, CC, CX3C and C chemokines. The CXC chemokines can be further classified into ELR+ and ELR− chemokines based on the presence or absence of the motif ‘glu-leu-arg (ELR motif)’ preceding the CXC sequence. The CXC chemokines bind to and activate their cognate chemokine receptors on neutrophils, lymphocytes, endothelial and epithelial cells. The CC chemokines act on several subsets of dendritic cells, lymphocytes, macrophages, eosinophils, natural killer cells but do not stimulate neutrophils as they lack CC chemokine receptors except murine neutrophils. There are approximately 50 chemokines and only 20 chemokine receptors, thus there is considerable redundancy in this system of ligand/receptor interaction.

Chemokines elaborated from the tumor and the stromal cells bind to the chemokine receptors present on the tumor and the stromal cells. The autocrine loop of the tumor cells and the paracrine stimulatory loop between the tumor and the stromal cells facilitate the progression of the tumor. Notably, CXCR2, CXCR4, CCR2 and CCR7 play major roles in tumorigenesis and metastasis. CXCR2 plays a vital role in angiogenesis and CCR2 plays a role in the recruitment of macrophages into the tumor microenvironment. CCR7 is involved in metastasis of the tumor cells into the sentinel lymph nodes as the lymph nodes have the ligand for CCR7, CCL21. CXCR4 is mainly involved in the metastatic spread of a wide variety of tumors.

Molecular Classes of Targeting Domains

Ligands and Receptors

In one embodiment, tumor or tumor-associated neovasculature targeting domains are ligands that bind to cell surface antigens or receptors that are specifically expressed on tumor cells or tumor-associated neovasculature or are overexpressed on tumor cells or tumor-associated neovasculature as compared to normal tissue. Tumors also secrete a large number of ligands into the tumor microenvironment that affect tumor growth and development. Receptors that bind to ligands secreted by tumors, including, but not limited to growth factors, cytokines and chemokines, including the chemokines provided above, are suitable for use in the disclosed fusion proteins. Ligands secreted by tumors can be targeted using soluble fragments of receptors that bind to the secreted ligands. Soluble receptor fragments are fragments polypeptides that may be shed, secreted or otherwise extracted from the producing cells and include the entire extracellular domain, or fragments thereof.

Single Polypeptide Antibodies

In another embodiment, tumor or tumor-associated neovasculature targeting domains are single polypeptide antibodies that bind to cell surface antigens or receptors that are specifically expressed on tumor cells or tumor-associated neovasculature or are overexpressed on tumor cells or tumor-associated neovasculature as compared to normal tissue. Single domain antibodies are described above with respect to coinhibitory receptor antagonist domains.

Fc Domains

In another embodiment, tumor or tumor-associated neovasculature targeting domains are Fc domains of immunoglobulin heavy chains that bind to Fc receptors expressed on tumor cells or on tumor-associated neovasculature. The Fc region as used herein includes the polypeptides containing the constant region of an antibody excluding the first constant region immunoglobulin domain. Thus Fc refers to the last two constant region immunoglobulin domains of IgA, IgD, and IgG, and the last three constant region immunoglobulin domains of IgE and IgM. In a preferred embodiment, the Fc domain is derived from a human or murine immunoglobulin. In a more preferred embodiment, the Fc domain is derived from human IgG1 or murine IgG2a including the C_{H}2 and C_{H}3 regions.

In one embodiment, the hinge, C_{H}2 and C_{H}3 regions of a human immunoglobulin Cγ1 chain are encoded by a nucleic acid having at least 80%, 85%, 90%, 95%, 99% or 100% sequence identity to:

| (SEQ ID NO: 44) | |
|---|---|
| gagcctaagt catgtgacaa gacccatacg tgcccaccct gtcccgctcc agaactgctg | 60 |
| gggggaccta gcgttttctt gttcccccca aagcccaagg acaccctcat gatctcacgg | 120 |
| actcccgaag taacatgcgt agtagtcgac gtgagccacg aggatcctga agtgaagttt | 180 |
| aattggtacg tggacggagt cgaggtgcat aatgccaaaa ctaaacctcg ggaggagcag | 240 |
| tataacagta cctaccgcgt ggtatccgtc ttgacagtgc tccaccagga ctggctgaat | 300 |
| ggtaaggagt ataaatgcaa ggtcagcaac aaagctcttc ccgccccaat tgaaaagact | 360 |
| atcagcaagg ccaagggaca accccgcgag ccccaggttt acacccttcc accttcacga | 420 |
| gacgagctga ccaagaacca ggtgtctctg acttgtctgg tcaaaggttt ctatccttcc | 480 |
| gacatcgcag tggagtggga gtcaaacggg cagcctgaga ataactacaa gaccacaccc | 540 |
| ccagtgcttg atagcgatgg gagctttttc ctctacagta agctgactgt ggacaaatcc | 600 |
| cgctggcagc agggaaacgt tttctcttgt agcgtcatgc atgaggccct ccacaaccat | 660 |
| tatactcaga aaagcctgag tctgagtccc ggcaaa | 696 |

The hinge, C_{H}2 and C_{H}3 regions of a human immunoglobulin Cγ1 chain encoded by SEQ ID NO:44 has the following amino acid sequence:

| (SEQ ID NO: 45) | |
|---|---|
| EPKSCDKTHT CPPCPAPELL GGPSVFLFPP KPKDTLMISR TPEVTCVVVD VSHEDPEVKF | 60 |
| NWYVDGVEVH NAKTKPREEQ INSTYRVVSV LTVLHQDWLN GKEYKCKVSN KALPAPIEKT | 120 |
| ISKAKGQPRE PQVYTLPPSR DELTKQVSL TCLVKGFYPS DIAVEWESNG QPENNYKTTP | 190 |
| PVLDSDGSFF LYSKLTVDKS RWQQGNVESC SVMHEALHNH YTQKSLSLSP GK | 232 |

In another embodiment, the hinge, C₁₋₁₂ and C_{H}3 regions of a murine immunoglobulin Cγ2a chain are encoded by a nucleic acid having at least 80%, 85%, 90%, 95%, 99% or 100% sequence identity to:

| (SEQ ID NO: 46) | |
|---|---|
| gagccaagag gtcctacgat caagccctgc ccgccttgta aatgccoagc tccaaatttg | 60 |
| ctgggtggac cgtcagtctt tatcttcccg ccaaagataa aggacgtctt gatgattagt | 120 |
| ctgagcccca tcgtgacatg cgttgtggtg gatgtttcag aggatgaccc cgacgtgcaa | 180 |
| atcagttggt tcgttaacaa cgtggaggtg cataccgctc aaacccagac ccacagagag | 240 |
| gattataaca gcaccctgcg ggtagtgtcc gccctgccga tccagcatca ggattggatg | 300 |
| agcgggaaag agttcaagtg taaggtaaac aacaaagatc tgccagcgcc gattgaacga | 360 |
| accattagca agccgaaagg gagcgtgcgc gcacctcagg tttacgtcct tcctccacca | 420 |
| gaagaggaga tgacgaaaaa gcaggtgacc ctgacatgca tggtaactga ctttatgcca | 480 |
| gaagatattt acgtggaatg gactaataac ggaaagacag agctcaatta caagaacact | 540 |
| gagcctgttc tggattctga tggcagctac tttatgtact ccaaattgag ggtcgagaag | 600 |
| aagaattggg tcgagagaaa cagttatagt tgctcagtgg tgcatgaggg cctccataat | 660 |
| catcacacca caaagtcctt cagccgaacg cccgggaaa | 699 |

The hinge, C_{H}2 and C_{H}3 regions of a murine immunoglobulin Cγ2a chain encoded by SEQ ID NO:46 has the following amino acid sequence:

| (SEQ ID NO: 47) | |
|---|---|
| EPRGPTIKPC PPCKCPAPNL LGGPSVFIFP PKIKDVLMIS LSPIVTCVVV DVSEDDPDVQ | 60 |
| ISWFVNNVEV HTAQTQTHRE DYNSTLRVVS ALPIQHQDWM SGKEFKCKVN NKDLPAPIER | 120 |
| TISKPKGSVR APQVYVLPPP EEEMTKKQVT LTCMVTDFMP EDIYVEWTNN GKTELNYKNT | 180 |
| EPVLDSDGSY FMYSKLRVEK KNWVERNSYS CSVVHEGLHN HHTTKSFSRT PGK | 233 |

In one embodiment, the Fc domain may contain one or more amino acid insertions, deletions or substitutions that enhance binding to specific Fc receptors that specifically expressed on tumors or tumor-associated neovasculature or are overexpressed on tumors or tumor-associated neovasculature relative to normal tissue. Suitable amino acid substitutions include conservative and non-conservative substitutions, as described above.

The therapeutic outcome in patients treated with rituximab (a chimeric mouse/human IgG1 monoclonal antibody against CD20) for non-Hodgkin's lymphoma or Waldenstrom's macroglobulinemia correlated with the individual's expression of allelic variants of Fcγ receptors with distinct intrinsic affinities for the Fc domain of human IgG1. In particular, patients with high affinity alleles of the low affinity activating Fc receptor CD16A (FcγRIIIA) showed higher response rates and, in the cases of non-Hodgkin's lymphoma, improved progression-free survival. In another embodiment, the Fc domain may contain one or more amino acid insertions, deletions or substitutions that reduce binding to the low affinity inhibitory Fc receptor CD32B (FcγRIIB) and retain wild-type levels of binding to or enhance binding to the low affinity activating Fc receptor CD16A (FcγRIIIA). In a preferred embodiment, the Fc domain contains amino acid insertions, deletions or substitutions that enhance binding to CD16A. A large number of substitutions in the Fc domain of human IgG1 that increase binding to CD16A and reduce binding to CD32B are known in the art and are described in Stavenhagen, et al., Cancer Res., 57(18):8882-90 (2007). Exemplary variants of human IgG1 Fc domains with reduced binding to CD32B and/or increased binding to CD16A contain F243L, R929P, Y300L, V305I or P296L substitutions. These amino acid substitutions may be present in a human IgG1 Fc domain in any combination. In one embodiment, the human IgG1 Fc domain variant contains a F243L, R929P and Y300L substitution. In another embodiment, the human IgG1 Fc domain variant contains a F243L, R929P, Y300L, V305I and P296L substitution.

Glycophosphatidylinositol Anchor Domain

In another embodiment, tumor or tumor-associated neovaseulature targeting domains are polypeptides that provide a signal for the posttranslational addition of a glycosylphosphatidylinositol (GPI) anchor. GPI anchors are glycolipid structures that are added posttranslationally to the C-terminus of many eukaryotic proteins. This modification anchors the attached protein in the outer leaflet of cell membranes. GPI anchors can be used to attach T cell receptor binding domains to the surface of cells for presentation to T cells. In this embodiment, the GPI anchor domain is C-terminal to the T cell receptor binding domain.

In one embodiment, the GPI anchor domain is a polypeptide that signals for the posttranslational addition of a GPI anchor when the polypeptide is expressed in a eukaryotic system. Anchor addition is determined by the GPI anchor signal sequence, which consists of a set of small amino acids at the site of anchor addition (the ω site) followed by a hydrophilic spacer and ending in a hydrophobic stretch (Low, FASEB J., 3:1600-1608 (1989)). Cleavage of this signal sequence occurs in the ER before the addition of an anchor with conserved central components (Low, FASEB J., 3:1600-1608 (1989)) but with variable peripheral moieties (Homans et al., Nature, 333:269-272 (1988)). The C-terminus of a GPI-anchored protein is linked through a phosphoethanolamine bridge to the highly conserved core glycan, mannose(α1-2)mannose(α1-6)mannose(α1-4)glucosamine(α1-6)myo-inositol. A phospholipid tail attaches the GPI anchor to the cell membrane. The glycan core can be variously modified with side chains, such as a phosphoethanolamine group, mannose, galactose, sialic acid, or other sugars. The most common side chain attached to the first mannose residue is another mannose. Complex side chains, such as the N-acetylgalactosamine-containing polysaccharides attached to the third mannose of the glycan core, are found in mammalian anchor structures. The core glucosamine is rarely modified. Depending on the protein and species of origin, the lipid anchor of the phosphoinositol ring is a diacylglycerol, an alkylacylglycerol, or a ceramide. The lipid species vary in length, ranging from 14 to 28 carbons, and can be either saturated or unsaturated. Many GPI anchors also contain an additional fatty acid, such as palmitic acid, on the 2-hydroxyl of the inositol ring. This extra fatty acid renders the GPI anchor resistant to cleavage by PI-PLC.

GPI anchor attachment can be achieved by expression of a fusion protein containing a GPI anchor domain in a eukaryotic system capable of carrying out GPI posttranslational modifications. GPI anchor domains can be used as the tumor or tumor vasculature targeting domain, or can be additionally added to fusion proteins already containing separate tumor or tumor vasculature targeting domains.

In another embodiment, GPI anchor moieties are added directly to isolated T cell receptor binding domains through an in vitro enzymatic or chemical process. In this embodiment, GPI anchors can be added to polypeptides without the requirement for a GPI anchor domain. Thus, GPI anchor moieties can be added to fusion proteins described herein having a T cell receptor binding domain and a tumor or tumor vasculature targeting domain. Alternatively, GPI anchors can be added directly to T cell receptor binding domain polypeptides without the requirement for fusion partners encoding tumor or tumor vasculature targeting domains.

2. Peptide or Polypeptide Linker Domain

Fusion proteins disclosed herein optionally contain a peptide or polypeptide linker domain that separates the costimulatory polypeptide domain from the antigen-binding targeting domain.

Hinge Region of Antibodies

In one embodiment, the linker domain contains the hinge region of an immunoglobulin. In a preferred embodiment, the hinge region is derived from a human immunoglobulin. Suitable human immunoglobulins that the hinge can be derived from include IgG, IgD and IgA. In a preferred embodiment, the hinge region is derived from human IgG.

In another embodiment, the linker domain contains a hinge region of an immunoglobulin as described above, and further includes one or more additional immunoglobulin domains. In one embodiment, the additional domain includes the Fc domain of an immunoglobulin. The Fc region as used herein includes the polypeptides containing the constant region of an antibody excluding the first constant region immunoglobulin domain. Thus Fc refers to the last two constant region immunoglobulin domains of IgA, IgD, and IgG, and the last three constant region immunoglobulin domains of IgE and IgM. In a preferred embodiment, the Fc domain is derived from a human immunoglobulin. In a more preferred embodiment, the Fc domain is derived from human IgG including the C_{H}2 and C_{H}3 regions.

In another embodiment, the linker domain contains a hinge region of an immunoglobulin and either the C_{H}1 domain of an immunoglobulin heavy chain or the C_{L} domain of an immunoglobulin light chain. In a preferred embodiment, the C_{H}1 or C_{L} domain is derived from a human immunoglobulin. The C_{L} domain may be derived from either a κ light chain or a λ light chain. In a more preferred embodiment, the C_{H}1 or C_{L} domain is derived from human IgG.

Amino acid sequences of immunoglobulin hinge regions and other domains are well known in the art.

Other Peptide/Polypeptide Linker Domains

Other suitable peptide/polypeptide linker domains include naturally occurring or non-naturally occurring peptides or polypeptides. Peptide linker sequences are at least 2 amino acids in length. Preferably the peptide or polypeptide domains are flexible peptides or polypeptides. A “flexible linker” herein refers to a peptide or polypeptide containing two or more amino acid residues joined by peptide bond(s) that provides increased rotational freedom for two polypeptides linked thereby than the two linked polypeptides would have in the absence of the flexible linker. Such rotational freedom allows two or more antigen binding sites joined by the flexible linker to each access target antigen(s) more efficiently. Exemplary flexible peptides/polypeptides include, but are not limited to, the amino acid sequences Gly-Ser, Gly-Ser-Gly-Ser (SEQ ID NO:48), Ala-Ser, Gly-Gly-Gly-Ser (SEQ ID NO:49), (Gly₄-Ser)₃ (SEQ ID NO:50), and (Gly₄-Ser)₄ (SEQ ID NO:51). Additional flexible peptide/polypeptide sequences are well known in the art.

3. Dimerization and Multimerization Domains

The fusion proteins disclosed herein optionally contain a dimerization or multimerization domain that functions to dimerize or multimerize two or more fusion proteins. The domain that functions to dimerize or multimerize the fusion proteins can either be a separate domain, or alternatively can be contained within one of the other domains (T cell costimulatory/coinhibitory receptor binding domain, tumor/tumor neovasculature antigen-binding domain, or peptide/polypeptide linker domain) of the fusion protein.

Dimerization Domains

A “dimerization domain” is formed by the association of at least two amino acid residues or of at least two peptides or polypeptides (which may have the same, or different, amino acid sequences). The peptides or polypeptides may interact with each other through covalent and/or non-covalent association(s). Preferred dimerization domains contain at least one cysteine that is capable of forming an intermolecular disulfide bond with a cysteine on the partner fusion protein. The dimerization domain can contain one or more cysteine residues such that disulfide bond(s) can form between the partner fusion proteins. In one embodiment, dimerization domains contain one, two or three to about ten cysteine residues. In a preferred embodiment, the dimerization domain is the hinge region of an immunoglobulin. In this particular embodiment, the dimerization domain is contained within the linker peptide/polypeptide of the fusion protein.

Additional exemplary dimerization domain can be any known in the art and include, but not limited to, coiled coils, acid patches, zinc fingers, calcium hands, a C_{H}1-C_{L} pair, an “interface” with an engineered “knob” and/or “protruberance” as described in U.S. Pat. No. 5,821,333, leucine zippers (e.g., from jun and/or fos) (U.S. Pat. No. 5,932,448), SH2 (src homology 2), SH3 (src Homology 3) (Vidal, et al., Biochemistry, 43, 7336-44 ((2004)), phosphotyrosine binding (PTB) (Zhou, et al., Nature, 378:584-592 (1995)), WW (Sudol, Prog. Biochys. Mol. Bio., 65:113-132 (1996)), PDZ (Kim, et al., Nature, 378: 85-88 (1995); Komau, et al., Science, 269:1737-1740 (1995)) 14-3-3, WD40 (Hu, et al., J Biol Chem., 273, 33489-33494 (1998)) EH, Lim, an isoleucine zipper, a receptor dimer pair (e.g., interleukin-8 receptor (IL-8R); and integrin heterodimers such as LFA-1 and GPIIIb/IIIa), or the dimerization region(s) thereof, dimeric ligand polypeptides (e.g. nerve growth factor (NGF), neurotrophin-3 (NT-3), interleukin-8 (IL-8), vascular endothelial growth factor (VEGF), VEGF-C, VEGF-D, PDGF members, and brain-derived neurotrophic factor (BDNF) (Arakawa, et al., J. Biol. Chem., 269(45): 27833-27839 (1994) and Radziejewski, et al., Biochem., 32(48): 1350 (1993)) and can also be variants of these domains in which the affinity is altered. The polypeptide pairs can be identified by methods known in the art, including yeast two hybrid screens. Yeast two hybrid screens are described in U.S. Pat. Nos. 5,283,173 and 6,562,576, both of which are herein incorporated by reference in their entireties. Affinities between a pair of interacting domains can be determined using methods known in the art, including as described in Katahira, et al., J. Biol. Chem., 277, 9242-9246 (2002)). Alternatively, a library of peptide sequences can be screened for heterodimerization, for example, using the methods described in WO 01/00814. Useful methods for protein-protein interactions are also described in U.S. Pat. No. 6,790,624.

Multimerization Domains

A “multimerization domain” is a domain that causes three or more peptides or polypeptides to interact with each other through covalent and/or non-covalent association(s). Suitable multimerization domains include, but are not limited to, coiled-coil domains. A coiled-coil is a peptide sequence with a contiguous pattern of mainly hydrophobic residues spaced 3 and 4 residues apart, usually in a sequence of seven amino acids (heptad repeat) or eleven amino acids (undecad repeat), which assembles (folds) to form a multimeric bundle of helices. Coiled-coils with sequences including some irregular distribution of the 3 and 4 residues spacing are also contemplated. Hydrophobic residues are in particular the hydrophobic amino acids Val, Ile, Leu, Met, Tyr, Phe and Trp. Mainly hydrophobic means that at least 50% of the residues must be selected from the mentioned hydrophobic amino acids.

The coiled coil domain may be derived from laminin. In the extracellular space, the heterotrimeric coiled coil protein laminin plays an important role in the formation of basement membranes. Apparently, the multifunctional oligomeric structure is required for laminin function. Coiled coil domains may also be derived from the thrombospondins in which three (TSP-1 and TSP-2) or five (TSP-3, TSP-4 and TSP-5) chains are connected, or from COMP (COMPcc) (Guo, et at., EMBO J., 1998, 17: 5265-5272) which folds into a parallel five-stranded coiled coil (Malashkevich, et al., Science, 274: 761-765 (1996)).

Additional coiled-coil domains derived from other proteins, and other domains that mediate polypeptide multimerization are known in the art and are suitable for use in the disclosed fusion proteins.

4. Exemplary Fusion Proteins

B7-DC

A representative murine PD-L2 fusion protein is encoded by a nucleic acid having at least 80%, 85%, 90%, 95%, 99% or 100% sequence identity to:

| (SEQ ID NO: 52) | |
|---|---|
| atgctgctcc tgctgccgat actgaacctg agcttacaac ttcatcctgt agcagcttta | 60 |
| ttcaccgtga cagcccctaa agaagtgtac accgtagacg tcggcagcag tgtgagcctg | 120 |
| gagtgcgatt ttgaccgcag agaatgcact gaactggaag ggataagagc cagtttgcag | 180 |
| aaggtagaaa atgatacgtc tctgcaaagt gaaagagcca ccctgctgga ggagcagctg | 240 |
| cccctgggaa aggctttgtt ccacatccct agtgtccaag tgagagattc ogggcagtac | 300 |
| cgttgcctgg tcatctgcgg ggccgcctgg gactacaagt acctgacggt gaaagtcaaa | 360 |
| gcttcttaca tgaggataga cactaggatc ctggaggttc caggtacagg ggaggtgcag | 420 |
| cttacctgcc aggctagagg ttatccccta gcagaagtgt cctggcaaaa tgtcagtgtt | 480 |
| cctgccaaca ccagccacat caggaccccc gaaggcctct accaggtcac cagtgttctg | 540 |
| cgcctcaagc ctcagcctag cagaaacttc agctgcatgt tctggaatgc tcacatgaag | 600 |
| gagctgactt cagccatcat tgaccctctg agtcggatgg aacccaaagt ccccagaacg | 660 |
| tgggagccaa gaggtcctac gatcaagccc tgcccgcctt gtaaatgccc agctccaaat | 720 |
| ttgctgggtg gaccgtcagt ctttatcttc ccgccaaaga taaaggacgt cttgatgatt | 780 |
| agtctgagcc ccatcgtgac atgcgttgtg gtggatgttt cagaggatga ccccgacgtg | 840 |
| caaatcagtt ggttcgttaa caacgtggag gtgcataccg ctcaaaccca gacccacaga | 900 |
| gaggattata acagcaccct gcgggtagtg tccgccctgc cgatccagca tcaggattgg | 960 |
| atgagcggga aagagttcaa gtgtaaggta aacaacaaag atctgccagc gccgattgaa | 1020 |
| cgaaccatta gcaagccgaa agggagcgtg cgcgcacctc aggtttacgt ccttcctcca | 1080 |
| ccagaagagg agatgacgaa aaagcaggtg accctgacat gcatggtaac tgactttatg | 1140 |
| ccagaagata tttacgtgga atggactaat aacggaaaga cagagctcaa ttacaagaac | 1200 |
| actgagcctg ttctggattc tgatggcagc tactttatgt actccaaatt gagggtcgag | 1260 |
| aagaagaatt gggtcgagag aaacagttat agttgctcag tggtgcatga gggcctccat | 1320 |
| aatcatcaca ccacaaagtc cttcagccga acgcccggga aatga | 1365 |

The murine PD-L2 fusion protein encoded by SEQ ID NO:52 has the following amino acid sequence:

| (SEQ ID NO: 53) | |
|---|---|
| MLLLLPILNL SLQLHPVAAL FTVTAPKEVY TVDVGSSVSL ECDFDRRECT ELEGIRASLQ | 60 |
| KVENDTSLQS ERATLLEEQL PLGKALFHIP SVQVRDSGQY RCLVICGAAW DYKYLTVKVK | 120 |
| ASYMRIDTRI LEVPGTGEVQ LTCQARGYPL AEVSWQNVSV PANTSHIRTP EGLYQVTSVL | 160 |
| RLKPQPSRNF SCMFWNAHMK ELTSAIIDPL SRMEPKVPRT WEPRGPTIKP CPPCKCPAPN | 240 |
| LLGGPSVFIF PPKIKDVLMI SLSPIVTCVV VDVSEDDPDV QISWFVNNVE VHTAQTQTHR | 300 |
| EDYNSTLRVV SALPIQHQDW MSGKEFKCKV NNKDLPAPIE RTISKPKGSV RAPQVYVLPP | 360 |
| PEEEMTKKQV TLTCMVTDFM PEDIYVEWTN NGKTELNYKN TEPVLDSDGS YFMYSKLRVE | 420 |
| KKNWVERNSY SCSVVHEGLH NHHTTKSFSR TPGK | 454 |

The amino acid sequence of the murine PD-L2 fusion protein of SEQ ID NO:53 without the signal sequence is:

| (SEQ ID NO: 54) | |
|---|---|
| LFTVTAPKEV YTVDVGSSVS LECDFDRREC TELEGIRASL QKVENDTSLQ SERATLLEEQ | 60 |
| LPLGKALFHI PSVQVRDSGQ YRCLVICGAA WDYKYLTVKV KASYMRIDTR ILEVPGTGEV | 120 |
| QLTCQARGYP LAEVSWQNVS VPANTSHIRT PEGLYQVTSV LRLKPQPSRN FSCMFWNAHM | 180 |
| KELTSAIIDP LSRMEPKVPR TWEPRGPTIK PCPPCKCPAP NLLGGPSVFI FPPKIKDVLM | 240 |
| ISLSPIVTCV VVDVSEDDPD VQISWFVNNV EVHTAQTQTH REDYNSTLRV VSALPIQHQD | 300 |
| WMSGKEFKCK VNNKDLPAPI ERTISKPKGS VRAPQVYVLP PPEEEMTKKQ VTLTCMVTDF | 360 |
| MPEDIYVEWT NNGKTELNYK NTEPVLDSDG SYFMYSKLRV EKKNWVERNS YSCSVVHEGL | 420 |
| HNHHTTKSFS RTPGK. | 435 |

A representative human PD-L2 fusion protein is encoded by a nucleic acid having at least 80%, 85%, 90%, 95%, 99% or 100% sequence identity to:

| (SEQ ID NO: 55) | |
|---|---|
| atgatctttc ttctcttgat gctgtctttg gaattgcaac ttcaccaaat cgcggccctc | 60 |
| tttactgtga ccgtgccaaa agaactgtat atcattgagc acgggtccaa tgtgaccctc | 120 |
| gaatgtaact ttgacaccgg cagccacgtt aacctggggg ccatcactgc cagcttgcaa | 180 |
| aaagttgaaa acgacacttc acctcaccgg gagagggcaa ccctcttgga ggagcaactg | 240 |
| ccattgggga aggcctcctt tcatatccct caggtgcagg ttcgggatga gggacagtac | 300 |
| cagtgcatta ttatctacgg cgtggcttgg gattacaagt atctgaccct gaaggtgaaa | 360 |
| gcgtcctatc ggaaaattaa cactcacatt cttaaggtgc cagagacgga cgaggtggaa | 420 |
| ctgacatgco aagccaccgg ctacccgttg gcagaggtca gctggcccaa cgtgagcgta | 480 |
| cctgctaaca cttctcattc taggacaccc gagggcctct accaggttac atccgtgctc | 540 |
| cgcctcaaac cgcccccagg ccggaatttt agttgcgtgt tttggaatac ccacgtgcga | 600 |
| gagctgactc ttgcatctat tgatctgcag tcccagatgg agccacggac tcatccaact | 660 |
| tgggaaccta aatcttgcga taaaactcat acctgtcccc cttgcccagc ccccgagctt | 720 |
| ctgggaggtc ccagtgtgtt tctgtttccc ccaaaaccta aggacacact tatgatatcc | 780 |
| cgaacgccgg aagtgacatg cgtggttgtg gacgtctcac acgaagaccc ggaggtgaaa | 840 |
| ttcaactggt acgttgacgg agttgaggtt cataacgcta agaccaagcc cagagaggag | 900 |
| caatacaatt ccacctatcg agtggttagt gtactgaccg ttttgcacca agactggctg | 960 |
| aatggaaaag aatacaagtg caaagtatca aacaaggctt tgcctgcacc catcgagaag | 1020 |
| acaatttcta aagccaaagg gcagcccagg gaaccgcagg tgtacacact cccaccatcc | 1080 |
| cgcgacgagc tgacaaagaa tcaagtatcc ctgacctgcc tggtgaaagg cttttaccca | 1140 |
| tctgacattg ccgtggaatg ggaatcaaat ggacaacctg agaacaacta caaaaccact | 1200 |
| ccacctgtgc ttgacagcga cgggtccttt ttcctgtaca gtaagctcac tgtcgataag | 1260 |
| tctcgctggc agcagggcaa cgtcttttca tgtagtgtga tgcacgaagc tctgcacaac | 1320 |
| cattacaccc agaagtctct gtcactgagc ccaggtaaat ga | 1362 |

The human PD-L2 fusion protein encoded by SEQ ID NO:55 has the following amino acid sequence:

| (SEQ ID NO: 56) | |
|---|---|
| MIFLLLMLSL ELQLHQIAAL FTVTVPKELY IIEHGSNVTL ECNFDTGSHV NLGAITASLQ | 60 |
| KVENDTSPHR ERATLLEEQL PLGKASFHIP QVQVRDEGQY QCIIIYGVAW DYKYLTLKVK | 120 |
| ASYRKINTHI LKVPETDEVE LTCQATGYPL AEVSWPNVSV PANTSHSRTP EGLYQVTSVL | 180 |
| RLKPPPGRNF SCVFWNTHVR ELTLASIDLQ SQMEPRTHPT WEPKSCDKTH TCPPCPAPEL | 240 |
| LGGPSVFLFP PKPKDTLMIS RTPEVTCVVV DVSHEDPEVK FNWYVDGVEV HNAKTKPREE | 300 |
| QYNSTYRVVS VLTVLHQDWL NGKEYKCKVS NKALPAPIEK TISKAKGQPR EPQVYTLPPS | 360 |
| RDELTKNQVS LTCLVKGFYP SDIAVEWESN GQPENNYKTT PPVLDSDGSF FLYSKLTVDK | 420 |
| SRWQQGNVFS CSVMHEALHN HYTQKSLSLS PGK | 453 |

The amino acid sequence of the human PD-L2 fusion protein of SEQ ID NO:56 without the signal sequence is:

| (SEQ ID NO: 57) | |
|---|---|
| LFTVTVPKEL YIIEHGSNVT LECNFDTGSH VNLGAITASL QKVENDTSPH RERATLLEEQ | 60 |
| LPLGKASFHI PQVQVRDEGQ YQCIIIYGVA WDYKYLTLKV KASYRKINTH ILKVPETDEV | 120 |
| ELTCQATGYP LAEVSWPNVS VPANTSHSRT PEGLYQVTSV LRLKPPPGRN FSCVFWNTHV | 180 |
| RELTLASIDL QSQMEPRTHP TWEPKSCDKT HTCPPCPAPE LLGGPSVFLF PPKPKDTLMI | 240 |
| SRTPEVTCVV VDVSHEDPEV KFNWYVDGVE VHNAKTKPRE EQYNSTYRVV SVLTVLHQDW | 300 |
| LNGKEYKCKV SNKALPAPIE KTISKAKGQP REPQVYTLPP SRDELTKNQV SLTCLVKGFY | 360 |
| PSDIAVEWES NGQPENNYKT TPPVLDSDGS FFLYSKLTVD KSRWQQGNVF SCSVMHEALH | 420 |
| NHYTQKSLSL SPGK. | 434 |

G. Isolated Nucleic Acid Molecules Encoding PD-1 Receptor Antagonists

Isolated nucleic acid sequences encoding PD-1 antagonist polypeptides, variants thereof and fusion proteins thereof are disclosed. As used herein, “isolated nucleic acid” refers to a nucleic acid that is separated from other nucleic acid molecules that are present in a mammalian genome, including nucleic acids that normally flank one or both sides of the nucleic acid in a mammalian genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule independent of other sequences (e.g., a chemically synthesized nucleic acid, or a cDNA or genomic DNA fragment produced by PCR or restriction endonuclease treatment), as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, a cDNA library or a genomic library, or a gel slice containing a genomic DNA restriction digest, is not to be considered an isolated nucleic acid.

Nucleic acids can be in sense or antisense orientation, or can be complementary to a reference sequence encoding a B7-DC, PD-L1, PD-1 or B7.1 polypeptide or variant thereof. Reference sequences include, for example, the nucleotide sequence of human B7-DC, human PD-L1 or murine PD-L2 and murine PD-L1 which are known in the art and discussed above.

Nucleic acids can be DNA, RNA, or nucleic acid analogs. Nucleic acid analogs can be modified at the base moiety, sugar moiety, or phosphate backbone. Such modification can improve, for example, stability, hybridization, or solubility of the nucleic acid. Modifications at the base moiety can include deoxyuridine for deoxythymidine, and 5-methyl-2′-deoxycytidine or 5-bromo-2′-deoxycytidine for deoxycytidine. Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev. 7:187-195; and Hyrup et al. (1996) Bioorgan. Med. Chem. 4:5-23. In addition, the deoxyphosphate backbone can be replaced with, for example, a phosphorothioate or phosphorodithioate backbone, a phosphoroamidite, or an alkyl phosphotriester backbone.

H. Vectors and Host Cells Expressing PD-1 Receptor Antagonists

Nucleic acids, such as those described above, can be inserted into vectors for expression in cells. As used herein, a “vector” is a replicon, such as a plasmid, phage, or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Nucleic acids in vectors can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen Life Technologies (Carlsbad, Calif.).

An expression vector can include a tag sequence. Tag sequences, are typically expressed as a fusion with the encoded polypeptide. Such tags can be inserted anywhere within the polypeptide including at either the carboxyl or amino terminus. Examples of useful tags include, but are not limited to, green fluorescent protein (GFP), glutathione S-transferase (GST), polyhistidine, c-myc, hemagglutinin, Flag™ tag (Kodak, New Haven, Conn.), maltose E binding protein and protein A. In one embodiment, the variant PD-L2 fusion protein is present in a vector containing nucleic acids that encode one or more domains of an Ig heavy chain constant region, preferably having an amino acid sequence corresponding to the hinge, C_{H2} and C_{H3} regions of a human immunoglobulin Cγ1 chain.

Vectors containing nucleic acids to be expressed can be transferred into host cells. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell such as a CHO cell) can be used to, for example, produce the PD-1 antagonist polypeptides described herein.

I. Antibody PD-1 Antagonists

Monoclonal and polyclonal antibodies that are reactive with epitopes of the PD-1 antagonists, or PD-1, are disclosed. Monoclonal antibodies (mAbs) and methods for their production and use are described in Kohler and Milstein, Nature 256:495-497 (1975); U.S. Pat. No. 4,376,110; Hartlow, E. et al., Antibodies: A Laboratory Manual, Cold Spring Harbor Laboratory Press, Cold Spring Harbor, N.Y., 1988); Monoclonal Antibodies and Hybridomas: A New Dimension in Biological Analyses, Plenum Press, New York, N.Y. (1980); H. Zola et al., in Monoclonal Hybridoma Antibodies: Techniques and Applications, CRC Press, 1982)).

Antibodies that bind to PD-1 and block signal transduction through PD-1, and which have a lower affinity than those currently in use, allowing the antibody to dissociated in a period of less than three months, two months, one month, three weeks, two weeks, one week, or a few days after administration, are preferred for enhancement, augmentation or stimulation of an immune response.

Immunoassay methods are described in Coligan, J. E. et al., eds., Current Protocols in Immunology, Wiley-Interscience, New York 1991 (or current edition); Butt, W. R. (ed.) Practical Immunoassay: The State of the Art, Dekker, N.Y., 1984; Bizollon, Ch. A., ed., Monoclonal Antibodies and New Trends in Immunoassays, Elsevier, N.Y., 1984; Butler, J. E., ELISA (Chapter 29), In: van Oss, C. J. et al., (eds), Immunochemistry, Marcel Dekker, Inc., New York, 1994, pp. 759-803; Butler, J. E. (ed.), Immunochemistry of Solid-Phase Immunoassay, CRC Press, Boca Raton, 1991; Weintraub, B., Principles of Radioimmunoassays, Seventh Training Course on Radioligand Assay Techniques, The Endocrine Society, March, 1986; Work, T. S. et al., Laboratory Techniques and Biochemistry in Molecular Biology, North Holland Publishing Company, NY, (1978) (Chapter by Chard, T., “An Introduction to Radioimmune Assay and Related Techniques”).

Anti-idiotypic antibodies are described, for example, in Idiotypy in Biology and Medicine, Academic Press, New York, 1984; Immunological Reviews Volume 79, 1984; Immunological Reviews Volume 90, 1986; Curr. Top. Microbial., Immunol. Volume 119, 1985; Bona, C. et al., CRC Crit. Rev. Immunol., pp. 33-81 (1981); Jerme, N K, Ann. Immunol. 125C:373-389 (1974); Jerne, N K, In: Idiotypes—Antigens on the Inside, Westen-Schnurr, I., ed., Editiones Roche, Basel, 1982, Urbain, J. et al., Ann. Immunol. 133D:179-(1982); Rajewsky, K. et al., Ann. Rev. Immunol. 1:569-607 (1983).

The antibodies may be xenogeneic, allogeneic, syngeneic, or modified forms thereof, such as humanized or chimeric antibodies. Antiidiotypic antibodies specific for the idiotype of a specific antibody, for example an anti-PD-L2 antibody, are also included. The term “antibody” is meant to include both intact molecules as well as fragments thereof that include the antigen-binding site and are capable of binding to a PD-1 antagonist epitope. These include, Fab and F(ab′)₂ fragments which lack the Fc fragment of an intact antibody, clear more rapidly from the circulation, and may have less non-specific tissue binding than an intact antibody (Wahl et al., J. Nuc. Med. 24:316-325 (1983)). Also included are Fv fragments (Hochman, J. et al. (1973) Biochemistry 12:1130-1135; Sharon, J. et al. (1976) Biochemistry 15:1591-1594). These various fragments are produced using conventional techniques such as protease cleavage or chemical cleavage (see, e.g., Rousseaux et al., Meth. Enzymol., 121:663-69 (1986)).

Polyclonal antibodies are obtained as sera from immunized animals such as rabbits, goats, rodents, etc. and may be used directly without further treatment or may be subjected to conventional enrichment or purification methods such as ammonium sulfate precipitation, ion exchange chromatography, and affinity chromatography.

The immunogen may include the complete PD-1 antagonist, PD-1, or fragments or derivatives thereof. Preferred immunogens include all or a part of the extracellular domain (ECD) of PD-1 antagonist or PD-1, where these residues contain the post-translation modifications, such as glycosylation. Immunogens including the extracellular domain are produced in a variety of ways known in the art, e.g., expression of cloned genes using conventional recombinant methods or isolation from cells of origin.

Monoclonal antibodies may be produced using conventional hybridoma technology, such as the procedures introduced by Kohler and Milstein, Nature, 256:495-97 (1975), and modifications thereof (see above references). An animal, preferably a mouse is primed by immunization with an immunogen as above to elicit the desired antibody response in the primed animal. B lymphocytes from the lymph nodes, spleens or peripheral blood of a primed, animal are fused with myeloma cells, generally in the presence of a fusion promoting agent such as polyethylene glycol (PEG). Any of a number of murine myeloma cell lines are available for such use: the P3-NS1/1-Ag4-1, P3-x63-k0Ag8.653, Sp2/0-Ag14, or HL1-653 myeloma lines (available from the ATCC, Rockville, Md.). Subsequent steps include growth in selective medium so that unfused parental myeloma cells and donor lymphocyte cells eventually die while only the hybridoma cells survive. These are cloned and grown and their supernatants screened for the presence of antibody of the desired specificity, e.g. by immunoassay techniques using PD-L2 or PD-L1 fusion proteins. Positive clones are subcloned, e.g., by limiting dilution, and the monoclonal antibodies are isolated.

Hybridomas produced according to these methods can be propagated in vitro or in vivo (in ascites fluid) using techniques known in the art (see generally Fink et al., Prog. Clin. Pathol., 9:121-33 (1984)). Generally, the individual cell line is propagated in culture and the culture medium containing high concentrations of a single monoclonal antibody can be harvested by decantation, filtration, or centrifugation.

The antibody may be produced as a single chain antibody or scFv instead of the normal multimeric structure. Single chain antibodies include the hypervariable regions from an Ig of interest and recreate the antigen binding site of the native Ig while being a fraction of the size of the intact Ig (Skerra, A. et al. Science, 240: 1038-1041 (1988); Pluckthun, A. et al. Methods Enzymol. 178: 497-515 (1989); Winter, G. et al. Nature, 349: 293-299 (1991)). In a preferred embodiment, the antibody is produced using conventional molecular biology techniques.

### III. Methods of Manufacture

A. Methods for Producing PD-1 Antagonist Polypeptides and Variants Thereof

Isolated PD-1 antagonist polypeptides, variants thereof, and fusion proteins thereof can be obtained by, for example, chemical synthesis or by recombinant production in a host cell. To recombinantly produce a PD-1 antagonist polypeptide, a nucleic acid containing a nucleotide sequence encoding the polypeptide can be used to transform, transduce, or transfect a bacterial or eukaryotic host cell (e.g., an insect, yeast, or mammalian cell). In general, nucleic acid constructs include a regulatory sequence operably linked to a nucleotide sequence encoding a PD-1 antagonist polypeptide. Regulatory sequences (also referred to herein as expression control sequences) typically do not encode a gene product, but instead affect the expression of the nucleic acid sequences to which they are operably linked.

Useful prokaryotic and eukaryotic systems for expressing and producing polypeptides are well know in the art include, for example, Escherichia coli strains such as BL-21, and cultured mammalian cells such as CHO cells.

In eukaryotic host cells, a number of viral-based expression systems can be utilized to express PD-1 antagonist polypeptides. Viral based expression systems are well known in the art and include, but are not limited to, baculoviral, SV40, retroviral, or vaccinia based viral vectors.

Mammalian cell lines that stably express variant costimulatory polypeptides can be produced using expression vectors with appropriate control elements and a selectable marker. For example, the eukaryotic expression vectors pCR3.1 (Invitrogen Life Technologies) and p91023(B) (see Wong et al. (1985) Science 228:810-815) are suitable for expression of variant costimulatory polypeptides in, for example, Chinese hamster ovary (CHO) cells, COS-1 cells, human embryonic kidney 293 cells, NIH3T3 cells, BHK21 cells, MDCK cells, and human vascular endothelial cells (HUVEC). Following introduction of an expression vector by electroporation, lipofection, calcium phosphate, or calcium chloride co-precipitation, DEAF dextran, or other suitable transfection method, stable cell lines can be selected (e.g., by antibiotic resistance to G418, kanamycin, or hygromycin). The transfected cells can be cultured such that the polypeptide of interest is expressed, and the polypeptide can be recovered from, for example, the cell culture supernatant or from lysed cells. Alternatively, a PD-1 antagonist polypeptide can be produced by (a) ligating amplified sequences into a mammalian expression vector such as pcDNA3 (Invitrogen Life Technologies), and (b) transcribing and translating in vitro using wheat germ extract or rabbit reticulocyte lysate.

PD-1 antagonist polypeptides can be isolated using, for example, chromatographic methods such as DEAE ion exchange, gel filtration, and hydroxylapatite chromatography. For example, a costimulatory polypeptide in a cell culture supernatant or a cytoplasmic extract can be isolated using a protein G column. In some embodiments, variant costimulatory polypeptides can be “engineered” to contain an amino acid sequence that allows the polypeptides to be captured onto an affinity matrix. For example, a tag such as c-myc, hemagglutinin, polyhistidine, or Flag™ (Kodak) can be used to aid polypeptide purification. Such tags can be inserted anywhere within the polypeptide, including at either the carboxyl or amino terminus. Other fusions that can be useful include enzymes that aid in the detection of the polypeptide, such as alkaline phosphatase. Immunoaffinity chromatography also can be used to purify costimulatory polypeptides.

Methods for introducing random mutations to produce variant polypeptides are known in the art. Random peptide display libraries can be used to screen for peptides which interact with a PD-1 receptors or ligands. Techniques for creating and screening such random peptide display libraries are known in the art (Ladner et al., U.S. Pat. No. 5,223,409; Ladner et al., U.S. Pat. No. 4,946,778; Ladner et al., U.S. Pat. No. 5,403,484 and Ladner et al., U.S. Pat. No. 5,571,698) and random peptide display libraries and kits for screening such libraries are available commercially.

B. Methods for Producing Isolated Nucleic Acid Molecules Encoding PD-1 Antagonist Polypeptides

Isolated nucleic acid molecules encoding PD-1 antagonist polypeptides can be produced by standard techniques, including, without limitation, common molecular cloning and chemical nucleic acid synthesis techniques. For example, polymerase chain reaction (PCR) techniques can be used to obtain an isolated nucleic acid encoding a variant costimulatory polypeptide. PCR is a technique in which target nucleic acids are enzymatically amplified. Typically, sequence information from the ends of the region of interest or beyond can be employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize a complementary DNA (cDNA) strand. Ligase chain reaction, strand displacement amplification, self-sustained sequence replication or nucleic acid sequence-based amplification also can be used to obtain isolated nucleic acids. See, for example, Lewis (1992) Genetic Engineering News 12:1; Guatelli et al. (1990) Proc. Natl. Acad. Sci. USA 87:1874-1878; and Weiss (1991) Science 254:1292-1293.

Isolated nucleic acids can be chemically synthesized, either as a single nucleic acid molecule or as a series of oligonucleotides (e.g., using phosphoramidite technology for automated DNA synthesis in the 3′ to 5′ direction). For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase can be used to extend the oligonucleotides, resulting in a single, double-stranded nucleic acid molecule per oligonucleotide pair, which then can be ligated into a vector. Isolated nucleic acids can also obtained by mutagenesis. PD-1 antagonist encoding nucleic acids can be mutated using standard techniques, including oligonucleotide-directed mutagenesis and/or site-directed mutagenesis through PCR. See, Short Protocols in Molecular Biology. Chapter 8, Green Publishing Associates and John Wiley & Sons, edited by Ausubel et al, 1992. Examples of amino acid positions that can be modified include those described herein.

### IV. Formulations

A. PD-1 Antagonist Formulations

### A. PD-1 Antagonist Formulations

Pharmaceutical compositions including PD-1 antagonists are provided. Pharmaceutical compositions containing peptides or polypeptides may be for administration by parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration. The compositions may also be administered using bioerodible inserts and may be delivered directly to an appropriate lymphoid tissue (e.g., spleen, lymph node, or mucosal-associated lymphoid tissue) or directly to an organ or tumor. The compositions can be formulated in dosage forms appropriate for each route of administration. Compositions containing antagonists of PD-1 receptors that are not peptides or polypeptides can additionally be formulated for enteral administration.

As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected. Therapeutically effective amounts of PD-1 antagonist cause an immune response to be activated, enhanced, augmented, or sustained, and/or overcome or alleviate T cell exhaustion and/or T cell anergy, and/or activate monocytes, macrophages, dendritic cells and other antigen presenting cells (“APCs”).

In a preferred embodiment, the PD-1 antagonist is administered in a range of 0.1-20 mg/kg based on extrapolation from tumor modeling and bioavailability. A most preferred range is 5-20 mg of PD-1 antagonist/kg. Generally, for intravenous injection or infusion, dosage may be lower than when administered by an alternative route.

1. Formulations for Parenteral Administration

In a preferred embodiment, the disclosed compositions, including those containing peptides and polypeptides, are administered in an aqueous solution, by parenteral injection. The formulation may also be in the form of a suspension or emulsion. In general, pharmaceutical compositions are provided including effective amounts of a peptide or polypeptide, and optionally include pharmaceutically acceptable diluents, preservatives, solubilizers, emulsifiers, adjuvants and/or carriers. Such compositions include sterile water, buffered saline (e.g., Tris-HCl, acetate, phosphate), pH and ionic strength; and optionally, additives such as detergents and solubilizing agents (e.g., TWEEN® 20, TWEEN 80, Polysorbate 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

2. Controlled Delivery Polymeric Matrices

Compositions containing one or more PD-1 antagonist or nucleic acids encoding the PD-1 antagonist can be administered in controlled release formulations. Controlled release polymeric devices can be made for long term release systemically following implantation of a polymeric device (rod, cylinder, film, disk) or injection (microparticles). The matrix can be in the form of microparticles such as microspheres, where peptides are dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel. The matrix can also be incorporated into or onto a medical device to modulate an immune response, to prevent infection in an immunocompromised patient (such as an elderly person in which a catheter has been inserted or a premature child) or to aid in healing, as in the case of a matrix used to facilitate healing of pressure sores, decubitis ulcers, etc.

Either non-biodegradable or biodegradable matrices can be used for delivery of PD-1 antagonist or nucleic acids encoding them, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5:13-22 (1987); Mathiowitz, et al., Reactive Polymers, 6:275-283 (1987); and Mathiowitz, et al., J. Appl. Polymer Sci., 35:755-774 (1988).

Controlled release oral formulations may be desirable. Antagonists of PD-1 inhibitory signaling can be incorporated into an inert matrix which permits release by either diffusion or leaching mechanisms, e.g., films or gums. Slowly disintegrating matrices may also be incorporated into the formulation. Another form of a controlled release is one in which the drug is enclosed in a semipermeable membrane which allows water to enter and push drug out through a single small opening due to osmotic effects. For oral formulations, the location of release may be the stomach, the small intestine (the duodenum, the jejunem, or the ileum), or the large intestine. Preferably, the release will avoid the deleterious effects of the stomach environment, either by protection of the active agent (or derivative) or by release of the active agent beyond the stomach environment, such as in the intestine. To ensure full gastric resistance an enteric coating (i.e., impermeable to at least pH 5.0) is essential. These coatings may be used as mixed films or as capsules such as those available from Banner Pharmacaps.

The devices can be formulated for local release to treat the area of implantation or injection and typically deliver a dosage that is much less than the dosage for treatment of an entire body. The devices can also be formulated for systemic delivery. These can be implanted or injected subcutaneously.

3. Formulations for Enteral Administration

Antagonists of PD-1 can also be formulated for oral delivery. Oral solid dosage forms are known to those skilled in the art. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the present proteins and derivatives. See, e.g., Remington's Pharmaceutical Sciences, 21st Ed. (2005, Lippincott, Williams & Wilins, Baltimore, Md. 21201) pages 889-964. The compositions may be prepared in liquid form, or may be in dried powder (e.g., lyophilized) form. Liposomal or polymeric encapsulation may be used to formulate the compositions. See also Marshall, K. In: Modern Pharmaceutics Edited by G. S. Banker and C. T. Rhodes Chapter 10, 1979. In general, the formulation will include the active agent and inert ingredients which protect the PD-1 antagonist in the stomach environment, and release of the biologically active material in the intestine.

Liquid dosage forms for oral administration, including pharmaceutically acceptable emulsions, solutions, suspensions, and syrups, may contain other components including inert diluents; adjuvants such as wetting agents, emulsifying and suspending agents; and sweetening, flavoring, and perfuming agents.

B. Vaccines Including PD-1 Receptor Antagonists

Vaccines require strong T cell responses to eliminate cancer cells. PD-1 antagonists described herein can be administered as a component of a vaccine to prevent an inhibitory signal to T cells. Vaccines disclosed herein include antigens, a source of PD-1 antagonist polypeptides and optionally adjuvants and targeting molecules. Sources of PD-1 antagonist polypeptides include any disclosed B7-DC, PD-L1, PD-1, or B7.1 polypeptides, fusion proteins thereof, variants thereof, nucleic acids encoding these polypeptides and fusion proteins, or variants thereof or host cells containing vectors that express PD-1 antagonist polypeptides.

1. Antigens

Antigens can be peptides, proteins, polysaccharides, saccharides, lipids, nucleic acids, or combinations thereof. The antigen can be derived from a transformed cell such as a cancer or leukemic cell and can be a whole cell or immunogenic component thereof. Suitable antigens are known in the art and are available from commercial government and scientific sources. The antigens can be purified or partially purified polypeptides derived from tumors or can be recombinant polypeptides produced by expressing DNA encoding the polypeptide antigen in a heterologous expression system. The antigens can be DNA encoding all or part of an antigenic protein. The DNA may be in the form of vector DNA such as plasmid DNA.

Antigens may be provided as single antigens or may be provided in combination. Antigens may also be provided as complex mixtures of polypeptides or nucleic acids.

The antigen can be a tumor antigen, including a tumor-associated or tumor-specific antigen, such as, but not limited to, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A1l, hsp70-2, KIAAO205, Mart2, Mum-1, 2, and 3, neo-PAP, myosin class I, OS-9, pml-RARα fusion protein, PTPRK, K-ras, N-ras, Triosephosphate isomeras, Bage-1, Gage 3, 4, 5, 6, 7, GnTV, Herv-K-mel, Lage-1, Mage-A1, 2, 3, 4, 6, 10, 12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, 13-Catenin, CDK4, Mum-1, p16, TAGE, PSMA, PSCA, CT7, telomerase, 43-9F, 5T4, 791Tgp72, α-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, and TPS.

2. Adjuvants

Optionally, the vaccines described herein may include adjuvants. The adjuvant can be, but is not limited to, one or more of the following: oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG; mineral-containing compositions (e.g., mineral salts, such as aluminium salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, keyhole limpet hemocyanin, and dinitrophenol).

Adjuvants may also include immunomodulators such as cytokines, interleukins (e.g., IL-1, IL-2, IL-4, IL-5, IL-6, IL-7, IL-12, etc.), interferons (e.g., interferon-.gamma.), macrophage colony stimulating factor, and tumor necrosis factor. In addition to PD-1 antagonists, other co-stimulatory molecules, including other polypeptides of the B7 family, may be administered. Such proteinaceous adjuvants may be provided as the full-length polypeptide or an active fragment thereof, or in the form of DNA, such as plasmid DNA.

### IV. Methods of Use

A. Activation of T Cells

PD-1 antagonists polypeptides and small molecules, variants thereof, fusion proteins thereof, nucleic acids encoding the PD-1 antagonist polypeptides and fusion proteins, or cells expressing the PD-1 antagonist polypeptides and fusions proteins can be used to prevent inactivation and/or prolong activation of T cells (i.e., increase antigen-specific proliferation of T cells, enhance cytokine production by T cells, stimulate differentiation ad effector functions of T cells and/or promote T cell survival) or overcome T cell exhaustion and/or anergy.

Preferred PD-1 antagonists include polypeptides that bind to endogenous PD-L1 or PD-L2 and reduce or inhibit PD-L1 and PD-L2 from interacting with the PD-1 receptor, such as PD-1 or B7-1 polypeptides. By reducing the interaction these ligands with PD-1, the negative signal transmitted by PD-1 is prevented or reduced. In the presence of suboptimal TCR signals, exogenous PD-L2 or PD-L1 polypeptides can stimulate increased proliferation and production of cytokines in vitro. Thus, PD-L2 and PD-L1 appear to also bind to T cell receptors other than PD-1. PD-1 antagonists that bind to and block the PD-1 receptor without transmitting the negative signal through PD-1 are also preferred. Examples of these antagonists include recombinant ligands of PD-1 such as PD-L2 and PD-L1 that do not trigger signal transduction with they bind to PD-1.

Methods for using PD-1 antagonist polypeptides include contacting a T cell with a PD-1 antagonist polypeptide in an amount effective to inhibit or reduce PD-1 signal transduction in the T cell. The contacting can be in vitro, ex vivo, or in vivo (e.g., in a mammal such as a mouse, rat, rabbit, dog, cow, pig, non-human primate, or a human).

The contacting can occur before, during, or after activation of the T cell. Typically, contacting of the T cell with a PD-1 antagonist polypeptide can be at substantially the same time as activation. Activation can be, for example, by exposing the T cell to an antibody that binds to the T cell receptor (TCR) or one of the polypeptides of the CD3 complex that is physically associated with the TCR. Alternatively, a T cell can be exposed to either an alloantigen (e.g., a MHC alloantigen) on, for example, an APC [e.g., an interdigitating dendritic cell (referred to herein as a dendritic cell), a macrophage, a monocyte, or a B cell] or an antigenic peptide produced by processing of a protein antigen by any of the above APC and presented to the T cell by MHC molecules on the surface of the APC. The T cell can be a CD4⁺ T cell or a CD8⁺ T cell.

In some embodiments, the PD-1 antagonist polypeptide can be administered directly to a T cell. Alternatively, an APC such as a macrophage, monocyte, interdigitating dendritic cell (referred to herein as a dendritic cell), or B cell can be transformed, transduced, or transfected with a nucleic acid containing a nucleotide sequence that encodes a PD-1 antagonist polypeptide, and the T cell can be contacted by the transformed, transduced, or transfected APC. The transformed, transduced, or transfected cell can be a cell, or a progeny of a cell that, prior to being transformed, transduced, or transfected, can be obtained from the subject to which it is administered, or from another subject (e.g., another subject of the same species).

The PD-1 antagonist polypeptide can be any PD-1 antagonist polypeptide described herein, including any of the disclosed amino acid alterations, polypeptide fragments, fusion proteins and combinations thereof.

If the activation is in vitro, the PD-1 antagonist polypeptide can be bound to the floor of a relevant culture vessel, or bead or other solid support, e.g. a well of a plastic microtiter plate.

In vitro application of the PD-1 antagonist polypeptide can be useful, for example, in basic scientific studies of immune mechanisms or for production of activated T cells for use in studies of T cell function or, for example, passive immunotherapy. Furthermore, PD-1 antagonist polypeptides can be added to in vitro assays (e.g., T cell proliferation assays) designed to test for immunity to an antigen of interest in a subject from which the T cells were obtained. Addition of PD-1 antagonist polypeptides to such assays would be expected to result in a more potent, and therefore more readily detectable, in vitro response. Moreover, PD-1 antagonist polypeptide, or an APC transformed, transfected, or transduced with a nucleic acid encoding such a polypeptide, can be used: (a) as a positive control in an assay to test for T cell enhancing activity by other molecules; or (b) in screening assays for compounds useful in inhibiting T costimulation (e.g., compounds potentially useful for treating autoimmune diseases or organ graft rejection).

B. Therapeutic Uses of PD-1 Antagonists

1. Treatment of Cancer

The PD-1 antagonists provided herein are generally useful in viva and ex vivo as immune response-stimulating therapeutics. In general, the disclosed antagonist compositions are useful for treating a subject having or being predisposed to any disease or disorder to which the subject's immune system mounts an immune response. The ability of PD-1 antagonists to inhibit or reduce PD-1 signal transaction enables a more robust immune response to be possible. The disclosed compositions are useful to stimulate or enhance immune responses involving T cells.

The disclosed PD-1 antagonists are useful for stimulating or enhancing an immune response in host for treating cancer by administering to subject an amount of a PD-1 antagonist effective to costimulate T cells in the subject. The types of cancer that may be treated with the provided compositions and methods include, but are not limited to, the following: bladder, brain, breast, cervical, colo-rectal, esophageal, kidney, liver, lung, nasopharangeal, pancreatic, prostate, skin, stomach, uterine, ovarian, testicular and hematologic.

Malignant tumors which may be treated are classified herein according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

2. Use of PD-1 Antagonists in Vaccines

The disclosed PD-1 antagonists or nucleic acids encoding the same may be administered alone or in combination with any other suitable treatment. In one embodiment the PD-1 antagonists can be administered in conjunction with, or as a component of, a vaccine composition. Suitable components of vaccine compositions are described above. The disclosed PD-1 antagonists can be administered prior to, concurrently with, or after the administration of a vaccine. In one embodiment the PD-1 antagonist composition is administered at the same time as administration of a vaccine.

The disclosed PD-1 antagonists compositions may be administered in conjunction with prophylactic vaccines, or therapeutic vaccines, which can be used to initiate or enhance a subject's immune response to a pre-existing antigen, such as a tumor antigen in a subject with cancer.

The desired outcome of a prophylactic, therapeutic or de-sensitized immune response may vary according to the disease, according to principles well known in the art. Similarly, immune responses against cancer, allergens or infectious agents may completely treat a disease, may alleviate symptoms, or may be one facet in an overall therapeutic intervention against a disease. For example, the stimulation of an immune response against a cancer may be coupled with surgical, chemotherapeutic, radiologic, hormonal and other immunologic approaches in order to affect treatment.

3. Adjuvant Therapy

The disclosed PD-1 antagonists or nucleic acids encoding the same may be use to overcome tolerance to antigens, and thereby treat cancer. Appropriate targeting of co-signaling pathways can lead to activation of T cells and overcome tolerance to tumor antigens. One embodiment provides administering an effective amount of a PD-1 antagonists or nucleic acids encoding the same to overcome antigen tolerance. Inhibition or reduction of PD-1 negative signaling can also amplify T cell responses and overall immunity following administration of a first therapeutic agent or a response to a poorly immunogenic antigen such as a tumor associated antigen. One embodiment provides passive administration of PD-1 antagonists or nucleic acids encoding the same following primary treatment, vaccination, or killing of the tumor (antibody-mediated, with chemotherapy or radiation or any combination thereof). The PD-1 antagonists are believed to enhance/boost the primary response resulting in a robust and long-lasting protective response to the tumor.

Treatment that is administered in addition to a first therapeutic agent to eradicate tumors is referred to as adjuvant therapy. Adjuvant treatment is given to augment the primary treatment, such as surgery or radiation, to decrease the chance that the cancer will recur. This additional treatment can result in an amplification of the primary response as evidenced by a more potent and/or prolonged response.

There are five main types of adjuvant therapy (note that some of these are also used as primary/monotherapy as well): 1.) Chemotherapy that uses drugs to kill cancer cells, either by preventing them from multiplying or by causing the cells to self-destruct, 2.) Hormone therapy to reduce hormone production and prevent the cancer from growing, 3.) Radiation therapy that uses high-powered rays to kill cancer cells, 4.) Immunotherapy that attempts to influence the body's own immune system to attack and eradicate any remaining cancer cells. Immunotherapy can either stimulate the body's own defenses (cancer vaccines) or supplement them (passive administration of antibodies or immune cells), or 5.) Targeted therapy that targets specific molecules present within cancer cells, leaving normal, healthy cells alone. For example, many cases of breast cancer are caused by tumors that produce too much of a protein called HER2. Trastuzumab (Herceptin) is used as adjuvant therapy that targets HER2 positive tumors.

Typically adjuvant treatments are co-administered or given in conjunction with primary treatments to induce multiple mechanisms and increase the chances of eradicating the tumor. Immunotherapy, and vaccines in particular, offer the unique advantages of inducing a sustained antitumor effect with exquisite specificity and with the ability to circumvent existing immune tolerance. It has been discovered that delaying “adjuvant therapy” maximizes the response and increases the chances of eradicating tumors.

In a preferred embodiment, PD-1 antagonists or nucleic acids encoding the same, as described herein, are administered following administration of a first therapeutic agent such as a cancer therapeutic agent. The timing of the administration of the adjuvant can range from day 0 to day 14 after the primary treatment and can include single or multiple treatments. In certain embodiments, the PD-1 antagonist is administered 1, 2, 3, 4, 5, 6, 7, 8, 9, 10, 11, 12, 13, or 14 days after administration of the primary treatment. The adjuvant is preferably administered systemically to the patient (IV, IM or SQ).

The choice of PD-1 antagonist for use to enhance the immune response may depend on the original mode of primary treatment. For example, the same PD-1 antagonist used in conjunction with chemotherapy may not work well with radiation treatment. Therefore specific combinations of therapeutics and PD-1 antagonist molecules may be required for optimum efficacy. The PD-1 antagonists may be optimized for the type of cancer, for example solid versus liquid tumor for example using affinity maturation.

PD-1 antagonists and nucleic acids encoding the same may be useful in the induction or enhancement of an immune response to tumors. For example cells can be engineered to carry a nucleic acid encoding a PD-1 antagonist as described herein, and then administered to a subject to traverse tumor-specific tolerance in the subject. Notably, ectopic expression of B7-1 in B7 negative murine tumor cells has been shown to induce T-cell mediated specific immunity accompanied by tumor rejection and prolonged protection to tumor challenge in mice. Cell gene therapy treatments utilizing B7-related factors may be modeled on animal experiments (see K. Dunussi-Joannopoulos et al., J. Pediatr. Hematol. Oncol. 19:356-340 (1997); K. Hiroishi et al., Gene Ther. 6:1988-1994 (1999); B. K. Martin et al., J. Immunol. 162:6663-6670 (1999); M. Kuiper et al., Adv. Exp. Med. Biol. 465:381-390 (2000)), or human phase I trial experiments (H. L. Kaufman et al. Hum. Gene Ther. 11:1065-1082 (2000)), which use B7-1 or B7-2 for gene transfer therapy.

Administration is not limited to the treatment of an existing tumor or infectious disease but can also be used to prevent or lower the risk of developing such diseases in an individual, i.e., for prophylactic use. Potential candidates for prophylactic vaccination include individuals with a high risk of developing cancer, i.e., with a personal or familial history of certain types of cancer.

Another embodiment provides a method for increasing the population of tumor infiltrating leukocytes in a subject by administering to the subject an effective amount of PD-1 antagonists or nucleic acids encoding the same to enhance activation of the subject's T cells.

C. Combination Therapies

The disclosed PD-1 antagonist compositions can be administered to a subject in need thereof alone or in combination with one or more additional therapeutic agents or combinations of the recited PD-1 antagonists. The additional therapeutic agents are selected based on the condition, disorder or disease to be treated. For example, PD-1 antagonists can be co-administered with one or more additional agents that function to enhance or promote an immune response.

1. Chemotherapeutic Agents

The PD-1 antagonist can also be combined with one or more additional therapeutic agents. Representative therapeutic agents include, but are not limited to chemotherapeutic agents and pro-apoptotic agents. Representative chemotherapeutic agents include, but are not limited to amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epirubicin, etoposide, fludarabine, fluorouracil, gemcitabine, hydroxycarbamide, idarubicin, ifosfamide, irinotecan, leucovorin, liposomal doxorubicin, liposomal daunorubicin, lomustine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, tegafur-uracil, temozolomide, teniposide, thiotepa, tioguanine, topotecan, treosulfan, vinblastine, vincristine, vindesine, vinorelbine, or a combination thereof. Representative pro-apoptotic agents include, but are not limited to fludarabinetaurosporine, cycloheximide, actinomycin D, lactosylceramide, 15d-PGJ(2) and combinations thereof.

In certain embodiments, more than one PD-1 antagonist can be used in combination to increase or enhance an immune response in a subject.

2. Fusion Proteins that Enhance Immune Responses

In other embodiments, the PD-1 antagonist may be co-administered with compositions containing other B7 family costimulatory molecules that enhance an immune response. The other B7 costimulatory polypeptide may be of any species of origin. In one embodiment, the costimulatory polypeptide is from a mammalian species. In a preferred embodiment, the costimulatory polypeptide is of murine or human origin. In one embodiment, the polypeptide is B7.1. Useful additional human B7 polypeptides have at least about 80, 85, 90, 95 or 100% sequence identity to the B7-2 polypeptide encoded by the nucleic acid having GenBank Accession Number U04343 or; the B7-H5 polypeptide encoded by the nucleic acid having GenBank Accession Number NP_{—}071436. B7-H5 is also disclosed in PCT Publication No. WO 2006/012232.

In a preferred embodiment, the additional B7 family molecules are provided as soluble fusion proteins as described herein. Soluble fusion proteins of B7 molecules that form dimers or multimers and have the ability to crosslink their cognate receptors and thereby function as receptor agonists.

In one embodiment, the first fusion partner is a fragment of a B7 family molecule, including, but not limited to B7-1, B7-2, or B7-H5. As used herein, a fragment of B7 molecule refers to any subset of the polypeptide that is a shorter polypeptide of the full length protein. Useful fragments are those that retain the ability to bind to their natural ligands. A B7 polypeptide that is a fragment of full-length B7 molecule typically has at least 20 percent, 30 percent, 40 percent, 50 percent, 60 percent, 70 percent, 80 percent, 90 percent, 95 percent, 98 percent, 99 percent, 100 percent, or even more than 100 percent of the ability to bind its natural ligand(s) as compared to full-length B7 molecules.

Fragments of 87 polypeptides include soluble fragments. Soluble B7 polypeptide fragments are fragments of B7 polypeptides that may be shed, secreted or otherwise extracted from the producing cells. Soluble fragments of B7 polypeptides include some or all of the extracellular domain of the receptor polypeptide, and lack some or all of the intracellular and/or transmembrane domains. In one embodiment, 87 polypeptide fragments include the entire extracellular domain of the B7 polypeptide. In other embodiments, the soluble fragments of B7 polypeptides include fragments of the extracellular domain that retain B7 biological activity. It will be appreciated that the extracellular domain can include 1, 2, 3, 4, or 5 amino acids from the transmembrane domain. Alternatively, the extracellular domain can have 1, 2, 3, 4, or 5 amino acids removed from the C-terminus, N-terminus, or both.

Generally, the B7 polypeptides or fragments thereof are expressed from nucleic acids that include sequences that encode a signal sequence. The signal sequence is generally cleaved from the immature polypeptide to produce the mature polypeptide lacking the signal sequence. It will be appreciated that the signal sequence of B7 polypeptides can be replaced by the signal sequence of another polypeptide using standard molecule biology techniques to affect the expression levels, secretion, solubility, or other property of the polypeptide. The signal sequence that is used to replace the signal sequence can be any known in the art.

B7 molecule fusion polypeptides include variant polypeptides that are mutated to contain a deletion, substitution, insertion, or rearrangement of one or more amino acids relative to the wild-type polypeptide sequence. Useful variant B7 fusion proteins are those that retain the ability to bind to receptor polypeptides. Variant B7 fusion polypeptides typically have at least 20 percent, 30 percent, 40 percent, 50 percent, 60 percent, 70 percent, 80 percent, 90 percent, 95 percent, 98 percent, 99 percent, 100 percent, or even more than 100 percent of the ability to bind to B7 receptor polypeptides as compared to full-length B7 molecules.

Variant B7-H5 fusion polypeptides can have any combination of amino acid substitutions, deletions or insertions. Variant polypeptides may contain one or more amino acid deletions, substitutions, insertions, or rearrangements within either or all of the first fusion partner, the second polypeptide, and/or the optional linker peptide sequence.

D. Combination Therapies

The PD-1 antagonist compositions can be administered to a subject in need thereof alone or in combination with one or more additional therapeutic agents. The additional therapeutic agents are selected based on the condition, disorder or disease to be treated. For example, aPD-1 antagonist can be co-administered with one or more additional agents that function to enhance or promote an immune response.

E. Adoptive Transfer

Adoptive T-cell therapy is a promising strategy for the treatment of patients with established tumors but is often limited to specific cancers where tumor-infiltrating lymphocytes, the source of T cells for ex vivo culture, can be obtained. One embodiment provides a method for treating cancer by administering an effective amount of an antagonist for PD-1 to inhibit or reduce PD-1 receptor mediated signal transduction in a tumor cell in combination with adoptive T-cell therapy of antigen specific T cells. The adoptive T-cell transfer can be administered to the subject prior to or following administration of the antagonist of PD-1 or added to the cells ex vivo.

Antigen-specific T-cell lines can be generated by in vitro stimulation with antigen followed by nonspecific expansion on CD3/CD28 beads. The ability to expand antigen-specific T cells can be assessed using IFN-gamma and granzyme B enzyme-linked immunosorbent spot. The phenotype of the resultant T-cell lines can be evaluated by flow cytometry, including the presence of FOXP3-expressing CD4(+) T cells. Amplification of antigen-specific T cell populations from Peripheral Blood Mononuclear Cells (PBMCs) is usually performed through repeated in-vitro stimulation with optimal length antigenic peptides in the presence of IL-2. Low doses of IL-2 (between 10 and 50 U/ml) have been used traditionally to avoid the activation/expansion of lymphokine-activated killer cells, as revealed in chromium release assays that were commonly employed to monitor specific T cell expansion. Concentrations of antigenic peptides can be 0.1-10 μM.

1. Tumor-Specific and Tumor-Associated Antigens

Antigens useful for expanding T cells can be obtained from biopsies of tumors from the subject to be treated. The antigens can be biochemically purified from the tumor biopsy. Alternatively, the antigens can be recombinant polypeptides. The antigen expressed by the tumor may be specific to the tumor, or may be expressed at a higher level on the tumor cells as compared to non-tumor cells. Antigenic markers such as serologically defined markers known as tumor associated antigens, which are either uniquely expressed by cancer cells or are present at markedly higher levels (e.g., elevated in a statistically significant manner) in subjects having a malignant condition relative to appropriate controls, are contemplated for use in certain embodiments.

Tumor-associated antigens may include, for example, cellular oncogene-encoded products or aberrantly expressed proto-oncogene-encoded products (e.g., products encoded by the neu, ras, trk, and kit genes), or mutated forms of growth factor receptor or receptor-like cell surface molecules (e.g., surface receptor encoded by the c-erb B gene). Other tumor-associated antigens include molecules that may be directly involved in transformation events, or molecules that may not be directly involved in oncogenic transformation events but are expressed by tumor cells (e.g., carcinoembryonic antigen, CA-125, melanoma associated antigens, etc.) (see, e.g., U.S. Pat. No. 6,699,475; Jager, et al., Int. J. Cancer, 106:817-20 (2003); Kennedy, et al., Int. Rev. Immunol., 22:141-72 (2003); Scanlan, et al. Cancer Immun., 4:1 (2004)).

Genes that encode cellular tumor associated antigens include cellular oncogenes and proto-oncogenes that are aberrantly expressed. In general, cellular oncogenes encode products that are directly relevant to the transformation of the cell, and because of this, these antigens are particularly preferred targets for immunotherapy. An example is the tumorigenic neu gene that encodes a cell surface molecule involved in oncogenic transformation. Other examples include the ras, kit, and trk genes. The products of proto-oncogenes (the normal genes which are mutated to form oncogenes) may be aberrantly expressed (e.g., overexpressed), and this aberrant expression can be related to cellular transformation. Thus, the product encoded by proto-oncogenes can be targeted. Some oncogenes encode growth factor receptor molecules or growth factor receptor-like molecules that are expressed on the tumor cell surface. An example is the cell surface receptor encoded by the c-erbB gene. Other tumor-associated antigens may or may not be directly involved in malignant transformation. These antigens, however, are expressed by certain tumor cells and may therefore provide effective targets. Some examples are carcinoembryonic antigen (CEA), CA 125 (associated with ovarian carcinoma), and melanoma specific antigens.

In ovarian and other carcinomas, for example, tumor associated antigens are detectable in samples of readily obtained biological fluids such as serum or mucosal secretions. One such marker is CA125, a carcinoma associated antigen that is also shed into the bloodstream, where it is detectable in serum (e.g., Bast, et al., N. Eng. J. Med., 309:883 (1983); Lloyd, et al., Int. J. Canc., 71:842 (1997). CA125 levels in serum and other biological fluids have been measured along with levels of other markers, for example, carcinoembryonic antigen (CEA), squamous cell carcinoma antigen (SCC), tissue polypeptide specific antigen (TPS), sialyl TN mucin (STN), and placental alkaline phosphatase (PLAP), in efforts to provide diagnostic and/or prognostic profiles of ovarian and other carcinomas (e.g., Sarandakou, et al., Acta Oncol., 36:755 (1997); Sarandakou, et al., Eur. J. Gynaecol. Oncol., 19:73 (1998); Meier, et al., Anticancer Res., 17(4B):2945 (1997); Kudoh, et al., Gynecol. Obstet. Invest., 47:52 (1999)). Elevated serum CA125 may also accompany neuroblastoma (e.g., Hirokawa, et al., Surg. Today, 28:349 (1998), while elevated CEA and SCC, among others, may accompany colorectal cancer (Gebauer, et al., Anticancer Res., 17(4B):2939 (1997)).

The tumor associated antigen, mesothelin, defined by reactivity with monoclonal antibody K-1, is present on a majority of squamous cell carcinomas including epithelial ovarian, cervical, and esophageal tumors, and on mesotheliomas (Chang, et al., Cancer Res., 52:181 (1992); Chang, et al., Int. J. Cancer, 50:373 (1992); Chang, et al., Int. J. Cancer, 51:548 (1992); Chang, et al., Proc. Natl. Acad. Sci. USA, 93:136 (1996); Chowdhury, et al., Proc. Natl. Acad. Sci. USA, 95:669 (1998)). Using MAb K-1, mesothelin is detectable only as a cell-associated tumor marker and has not been found in soluble form in serum from ovarian cancer patients, or in medium conditioned by OVCAR-3 cells (Chang, et al., Int. J. Cancer, 50:373 (1992)). Structurally related human mesothelin polypeptides, however, also include tumor-associated antigen polypeptides such as the distinct mesothelin related antigen (MRA) polypeptide, which is detectable as a naturally occurring soluble antigen in biological fluids from patients having malignancies.

A tumor antigen may include a cell surface molecule. Tumor antigens of known structure and having a known or described function (see above).

2. Antigens Associated with Tumor Neovasculature

Protein therapeutics can be ineffective in treating tumors because they are inefficient at tumor penetration. Tumor-associated neovasculature provides a readily accessible route through which protein therapeutics can access the tumor. In another embodiment the fusion proteins contain a domain that specifically binds to an antigen that is expressed by neovasculature associated with a tumor.

The antigen may be specific to tumor neovasculature or may be expressed at a higher level in tumor neovasculature when compared to normal vasculature. Exemplary antigens that are over-expressed by tumor-associated neovasculature as compared to normal vasculature include, but are not limited to, VEGF/KDR, Tie2, vascular cell adhesion molecule (VCAM), endoglin and α₅β₃ integrin/vitronectin. Other antigens that are over-expressed by tumor-associated neovasculature as compared to normal vasculature are known to those of skill in the art and are suitable for targeting by the disclosed fusion proteins.

### EXAMPLES

The present invention may be further understood by reference to the following non-limiting examples.

### Example 1

### B7-DC Binding to PD-1

PD-1 binding activity of human B7-DC-Ig was assessed by ELISA. 96-well ELISA plates were coated with 100 μL 0.75 ug/mL recombinant human PD-1/Fc (R&D Systems) diluted in BupH Carbonate/Bicarbonate pH 9.4 buffer (Pierce) for 2 hours and then blocked with BSA solution (Jackson ImmunoResearch) for 90-120 minutes. Serially diluted human B7-DC-Ig as well as human IgG1 isotype control were allowed to bind for 90 minutes. Bound B7-DC-Ig was detected using 100 uL of 0.5 ug/mL biotin conjugated anti-human B7-DC clone MIH18 (eBioscience) followed by 1:1000 diluted HRP-Streptavidin (BD Bioscience) and TMB substrate (BioFX). Absorbance at 450 nm was read using a plate reader (Molecular Devices) and data were analyzed in SoftMax using a 4-parameter logistic fit.

PD-1 binding activity of murine B7-DC-Ig was assessed by ELISA. 96-well ELISA plates were coated with 100 μL 0.75 ug/mL recombinant mouse PD-1/Fc (R&D Systems) diluted in BupH Carbonate/Bicarbonate pH 9.4 buffer (Pierce) for 2 hours and then blocked with BSA solution (Candor-Bioscience) for 90 minutes. Serially diluted murine B7-DC-Ig (wild type, as well as D111S and K113S mutants that were selected for reduced binding to PD-1) as well as murine IgG2a isotype control were allowed to bind for 90 minutes. Bound B7-DC-Ig was detected using 100 uL of 0.25 ug/mL biotin conjugated anti-mouse B7-DC clone 112 (eBioscience) followed by 1:2000 diluted HRP-Streptavidin (BD Bioscience) and TMB substrate (BioFX). Absorbance at 450 nm was read using a plate reader (Molecular Devices) and data were analyzed in SoftMax using a 4-parameter logistic fit.

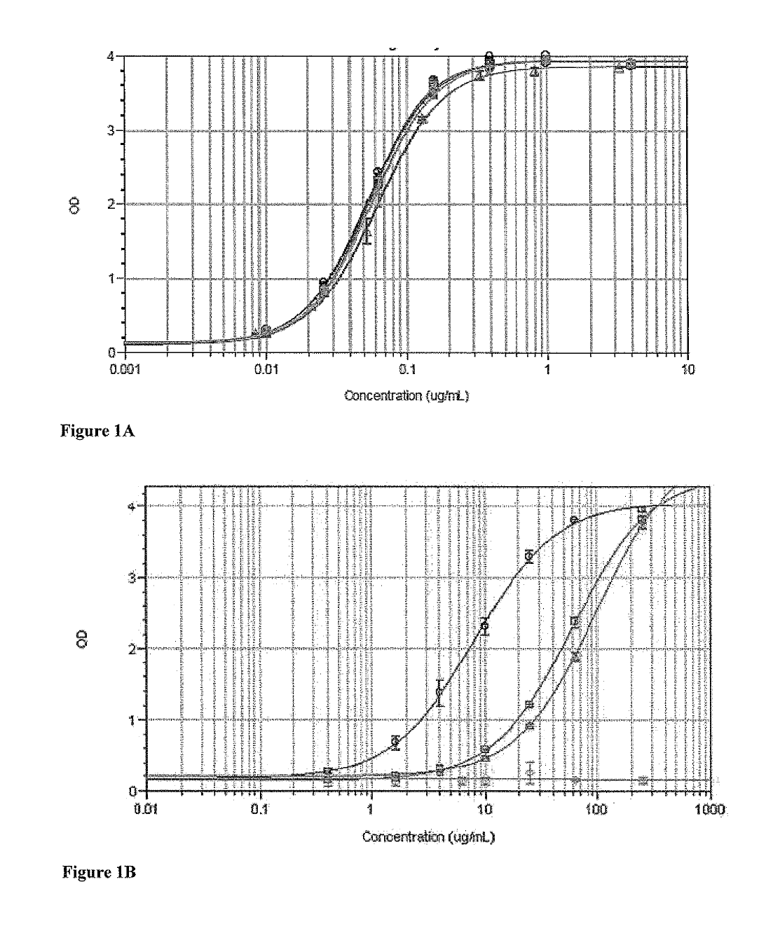

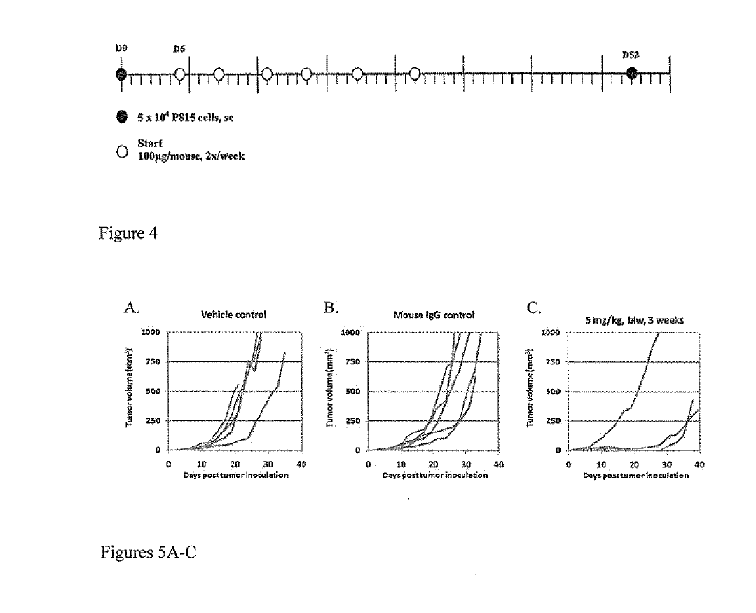

FIGS. 1A and 1B show line graphs of OD450 versus amount of B7-DC-Ig (ug/ml) in a PD-1 binding ELISA. FIG. 4A 1A shows binding of four different lots of human B7-DC-Ig. FIG. 4B 1B shows binding of wild type murine B7-DC-Ig (circle), the DS mutant (B7-DC-Ig with the D111S substitution; triangle) and KS mutant (B7-DC-Ig with the K113S substitution; square), and murine IgG2a isotype control (diamond).

### Example 2

### B7-DC Binding to PD-1 Expressing CHO Cells

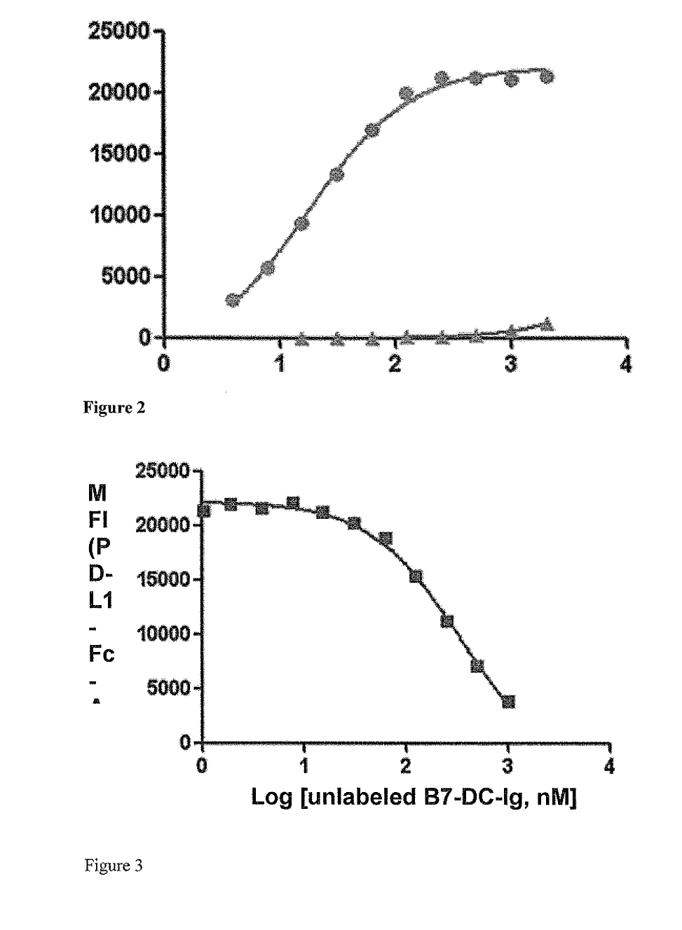

B7-DC-Ig was first conjugated with allophycocyanin (APC) and then incubated at various concentrations with a CHO cell line constitutively expressing PD-1 or parent CHO cells that do not express PD-1. Binding was analyzed by flow cytometry. FIG. 2 shows the median fluorescence intensity (MFI) of B7-DC-Ig-APC (y-axis) as a function of the concentration of probe (x-axis). B7-DC-Ig-APC binds to CHO.PD-1 cells (solid circle) but not untransfected CHO cells (gray triangle).

### Example 3

### B7-DC-Ig Competes with B7-H1 for Binding to PD-1

B7-H1-Ig was first conjugated with allophycocyanin (APC). Unlabeled B7-DC-Ig at various concentrations was first incubated with a CHO cell line constitutively expressing PD-1 before adding B7-H1-Ig-APC to the probe and cell mixture. FIG. 3 shows the median fluorescence intensity (MFI) of B7-H1-Ig-APC (y-axis) as a function of the concentration of unlabeled B7-DC-Ig competitor (x-axis) added. As the concentration of unlabeled B7-DC-Ig is increased the amount of B7-H1-Ig-APC bound to CHO cells decreases, demonstrating that B7-DC-Ig competes with B7-H1 for binding to PD-1.

### Example 4

### P815 Mastocytoma Model

The in vivo activity of murine B7-DC-Ig was tested in the P815 mastocytoma tumor model. P815 mastocytoma cells were derived from DBA/2 mice after methylcholanthrene (MCA) treatment. Injection of 5×10⁴ cells SC can result in mortality approximately 35 days post tumor inoculation.

DBA/2 mice (6-10 weeks of age, females) were first challenged with 5×10⁴ live P815 cells injected SC in the flank. Six days later, the mice were treated with murine B7-DC-Ig via IP injection. The dosing regimen, shown in FIG. 4, was 100 ug of murine B7-DC-Ig per injection (approximately 5 mg/kg), 2 times per week, up to 6 doses. Control groups were treated with vehicle only or with murine IgG. Tumor size was measured with digital calipers every 2-3 days. Mice were euthanized and defined as dead when their tumor size reached or exceeded 1000 mm³, according to protocols approved by the Institutional Animal Care and Use Committee (IACUC) of the American Red Cross (ARC; the site of Amplimmune's vivarium). Surviving tumor free mice were re-challenged with P815 tumor cells on Day 52.

As shown in Table 1 and FIG. 4, all of the mice treated with vehicle or control mouse IgG required euthanasia by Day 38 because their tumor volumes reached the IACUC limit. Four of 5 murine B7-DC-Ig treated mice responded to treatment: tumor was eradicated in two mice and two additional mice showed delayed tumor growth during murine B7-DC-Ig treatment.

TABLE 1
P815 tumor model results.

| | | # Tumor | # Tumor | # Tumor |
|---|---|---|---|---|
| Group | Treatment | free | <500 mm³ | ≧500 mm³ |
| A | Vehicle control | 0 | 0 | 5 |
| B | Mouse IgG control | 0 | 0 | 5 |
| C | Murine B7-DC-Ig | 2 | 2 | 1 |
| | (5 mg/kg IP biw 3 weeks | | | |
| | starting Day 6) | | | |

FIGS. 5A-C show tumor eradication in mice using murine B7-DC-Ig. The tumor-free mice were then re-challenged with 5×10⁴ P815 cells administered to the flank opposite the primary inoculation site on Day 52. The mice remained tumor free through 74 days after the primary inoculation, while all naïve mice challenged with P815 cells developed tumors. This suggests that mice inoculated with P815 cells and treated with murine B7-DC-Ig developed long-term immunity against P815 mastocytoma.

### Rationale of the CTX+B7-DC-Ig Regimen

Murine B7-DC-Ig alone is effective in the P815 model, which is considered relatively immunogenic, but shows minimal activity against more aggressive, less immunogenic tumor types. We expect that it will also be difficult to promote an effective anti-tumor immune response in human cancer patients.

To improve the activity of B7-DC-Ig and its murine analog, CTX was incorporated in the treatment regimen based on studies demonstrating that a low dose of CTX can safely and effectively augment the activity of cancer immunotherapies. Doses of 100-300 mg/m² in human or 20-200 mg/kg in mouse are typically used. These doses are sub-therapeutic and do not have direct anti-tumor activity.

In cancer patients and in murine syngeneic and genetic models of cancer, low doses of CTX lead to selective depletion of Treg. Treg are relatively abundant in the tumor microenvironment and play a major role in suppressing anti-tumor immune responses. Administration of CTX prior to treatment with an antigenic stimulus, vaccine, or cytokine, promotes a more functional anti-tumor immune response leading to enhanced tumor eradication. A number of clinical trials of low-dose CTX administered as a single agent or in combination with cancer vaccines or cytokines were extremely well tolerated and showed evidence of immune enhancement as well as clinical efficacy.

Based on the extensive experience and safety using low-dose CTX, its characterization as an immunopotentiator, as well as data generated at Amplimmune, B7-DC-Ig should be administered in a standard regimen consisting of CTX administration followed by B7-DC-Ig administration. A dose of 100 mg/kg was used in animal studies. CTX is delivered 24 hours before B7-DC-Ig or murine B7-DC-Ig treatment is initiated. Alternate dosing regimens such as metronomic CTX can be used.

### Example 6

### CT26 Tumor Model

Mouse colorectal tumor cell line, CT26, was obtained from ATCC. A master cell bank at Passage 4 was generated following ATCC guidelines. Cells were tested and confirmed no mycoplasma and other pathogen contamination.

One vial of tumor cells was thawed from the cryopreserved stocks and grown for two passages prior to inoculation.

CT26 cells were split at 1:5 dilution with 30 mL complete medium (RPMI+10% FBS, 2 mM L-Glu, and 1×P/S) for two days culture or at 1:10 dilution with 30 ml complete medium for 3 days culture.

CT26 cells were harvested by aspirating medium, rinsing the flask with 5 mL PBS, adding 5 mL trypsin, incubating at 37° C. for 2 min, and then neutralizing with 10 mL complete medium. After centrifuge at 600×g (˜1000 rpm) for 5 min, media was aspirated and the cell pellet was resuspended by pipetting with 10 ml plain RPMI. This wash step was repeated for three times.

Cell number and viability of the inoculated cells were analyzed by trypan blue dye staining with proper dilution (e.g. 1:5 dilution, 10 μL cells+40 μL trypan blue) and confirmed by NOVA cell count during the last wash step. Cell viability generally was greater than 95% for inoculation.

CT26 cells were diluted to 6.7×105 cells/mL for initial inoculation with plain RPMI and stored on ice. Typically each mouse was inoculated with 150 □L (1×105 cells).

On Day 9, all the tumor-bearing mice were first grouped into a rat cage and randomly divided the mice to experimental groups. CTX solution was reconstituted by 1×PBS to 4 mg/mL. Mice were intraperitoneally (IP) injected with 0.5 mL of CTX solution resulting in 2 mg for a 20 gram mouse, i.e. 100 mg/kg.

On Day 10, mice were IP injected with 0.5 mL of B7-DC-Ig (0.2 mg/mL) resulting in 0.1 mg for a 20 gram mouse, i.e. 5 mg/kg. The same dose was given 2 time a week for 4 weeks, total 8 doses. Tumor growth were monitored by measuring the tumor twice weekly, starting on the day when giving B7-DC-Ig via a digital caliper. Tumor volume was calculated as following:

Tumor volume=π(Dshort)2×(Dlong)/6=˜0.52×(Dshort)2×(Dlong)

Mice were euthanized and taken off the study if the tumor volume reached 2000 mm3 or if there were skin ulcers and infections at the tumor inoculation site.

### Example 5

### Combination of Cyclophosphamide and B7-DC-Ig can Eradicate Established Tumors

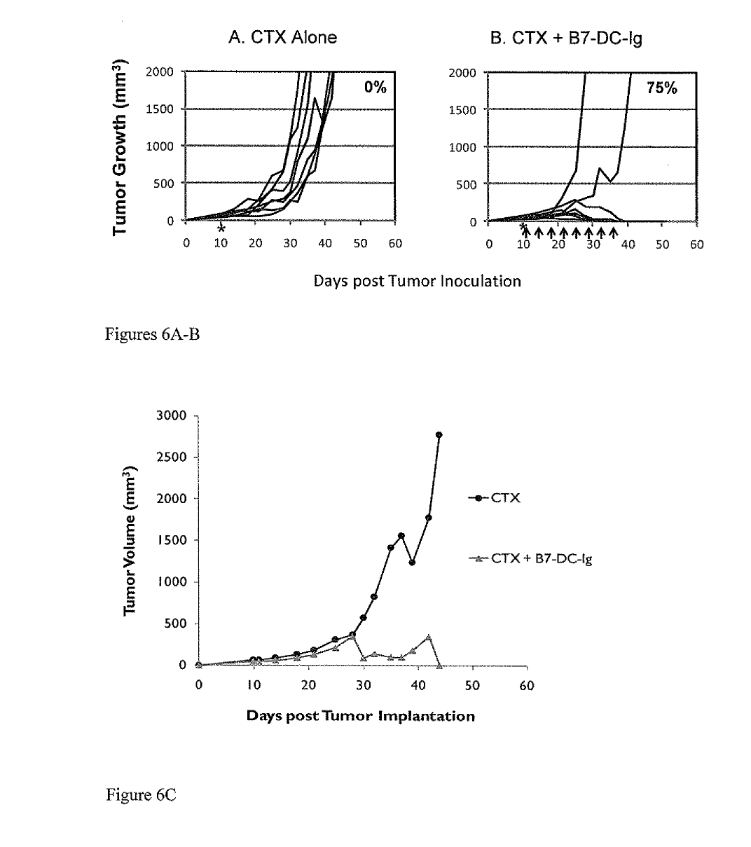

Balb/C mice at age of 9 to 11 weeks were implanted subcutaneously with 1.0×105 CT26 colorectal tumor cells as described above. On day 10 post tumor implantation, mice received 100 mg/kg of cyclophosphamide. B7-DC-Ig treatment started 1 day later, on day 11. Mice were treated with 100 ug of B7-DC-Ig, 2 doses per week, for 4 weeks and total 8 doses. 75% of the mice that received the CTX+B7-DC-Ig treatment regimen eradicated the established tumors by Day 44, whereas all mice in the control CTX alone group died as a result of tumor growth or were euthanized because tumors exceeded the sizes approved by IACUC (results shown in FIG. 6). These results demonstrate the effectiveness of the treatment regimen on established tumors and not mere prophylaxis.

### Example 6

### Combination of Cyclophosphamide and B7-DC-Ig can Eradicate Established Tumors and Protect Against Tumor Re-Challenge

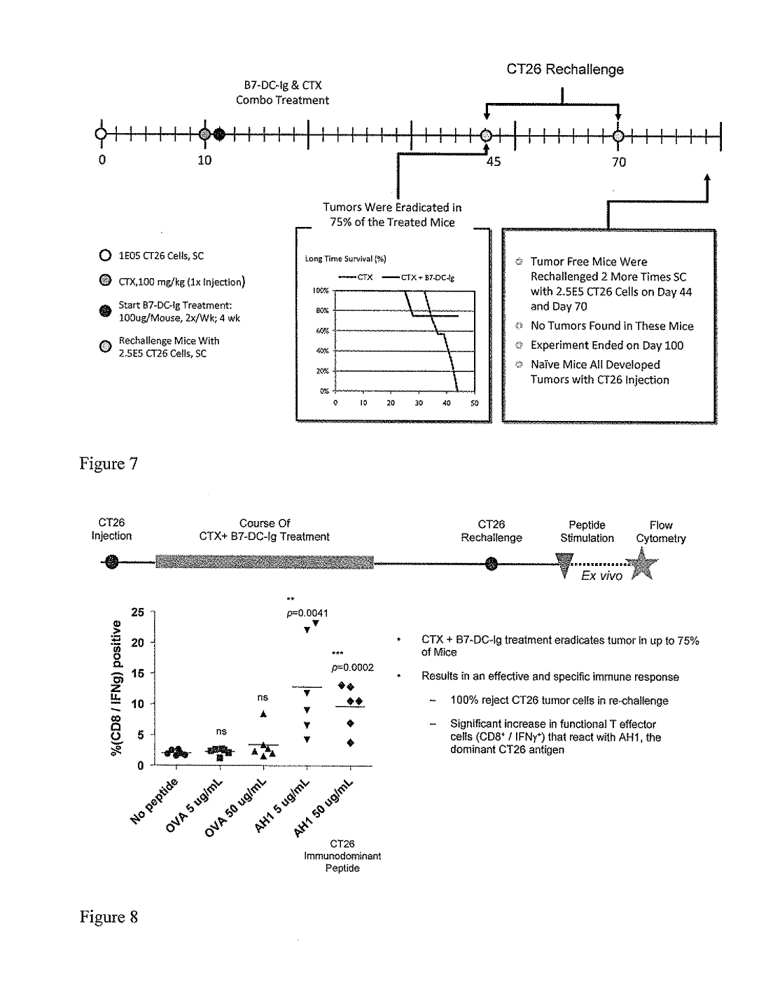

Mice eradicated established CT26 colorectal tumors from the above described experiment were rechallenged with 1×10⁵ CT26 cells on Day 44 and Day 70. No tumors grew out from the rechallenge suggesting they had developed long term anti-tumor immunity from the cyclophosphamide and B7-DC-Ig combination treatment. All mice in the vehicle control group developed tumors (results shown in FIG. 7). These results show the effectiveness of the treatment regimen on established tumors and that the cyclophosphamide and B7-DCIg combination treatment resulted in memory responses to tumor antigens.

### Example 7

### Combination of Cyclophosphamide and B7-DC-Ig can Generate Tumor Specific, Memory Cytotoxic T Lymphocytes

Mice eradicated established CT26 colorectal tumors from the above described experiment were rechallenged with 2.5×105 CT26 cells on Day 44. Seven days later, mouse spleens were isolated. Mouse splenocytes were pulsed with 5 or 50 ug/mL of ovalbumin (OVA) or AH1 peptides for 6 hours in the presence of a Golgi blocker (BD BioScience). Memory T effector cells were analyzed by assessing CD8+/IFNγ+ T cells. Results in FIG. 8 show that there were significant amount of CT26 specific T effector cells in the CT26 tumor-eradicated mice.

Unless defined otherwise, all technical and scientific terms used herein have the same meanings as commonly understood by one of skill in the art to which the disclosed invention belongs.

Those skilled in the art will recognize, or be able to ascertain using no more than routine experimentation, many equivalents to the specific embodiments of the invention described herein. Such equivalents are intended to be encompassed by the following claims.

We claim:

1. A method of modulating an immune response comprising administering an effective amount a PD-1 antagonist to induce, augment, or enhance an immune response against a tumor, wherein the dose of the molecule, the timing of administration of the molecule and/or the affinity of the molecule allows for intermittent access of a ligand to the PD-1 receptor.

2. The method of claim 1 wherein the PD-1 antagonist inhibits or reduces binding of endogenous PD-L1 to PD-1.

3. The method of claim 1 wherein the PD-1 antagonist inhibits or reduces binding of endogenous PD-L2 to PD-1.

4. The method of claim 1 wherein the PD-1 antagonist binds to PD-1.

5. The method of claim 1 wherein the PD-1 antagonist is selected from the group consisting of PD-1, PD-L1, PD-L2, B7.1, and fragments thereof.

6. The method of claim 1 wherein the molecule binds to PD-1 or a ligand thereof for three months or less after in vivo administration.

7. The method of claim 1 wherein more than one PD-1 antagonist is administered.

8. The method of claim 1, wherein the tumor is from a cancer selected from the group consisting of: bladder, brain, breast, cervical, colo-rectal, esophageal, kidney, liver, lung, nasopharangeal, pancreatic, prostate, skin, stomach, uterine, ovarian, testicular and hematologic.

9. The method of claim 1 further comprising administering a tumor antigen in combination with the PD-1 antagonist to enhance an immune response against the tumor.

10. The method of claim 1, wherein the PD-1 antagonist is a fusion protein of a PD-1 ligand.

11. The method of claim 10, wherein the fusion protein comprises the extracellular domain of PD-L2 or a fragment thereof capable of binding to PD-1.

12. The method of claim 11 wherein the fusion protein has an amino acid sequence according to SEQ ID NO:57.

13. The method of claim 1, further comprising administering with the PD-1 antagonist an additional active agent selected from the group consisting of immunomodulators, agents that deplete or inhibit the function of Tregs, and costimulatory molecules.

14. The method of claim 17, wherein the additional active agent is an agent that depletes or inhibits the function of CD4+CD25+ Tregs.

15. The method of claim 17, wherein the agent that depletes or inhibits the function of CD4+CD25+ Tregs is cyclophosphamide.

16. The method of claim 1 for enhancing antigen presenting cell function comprising contacting APCs with a PD-1 antagonist in an amount effective to inhibit, reduce, or block PD-1 signal transduction in the APCs or enhance clearance of diseased.

17. A composition comprising an effective amount of a PD-1 receptor antagonist to bind to a ligand of a PD-1 receptor in vivo and reduce or inhibit PD-1 receptor signal transduction.

18. The composition of claim 17 wherein the PD-1 antagonist comprises a B7-DC polypeptide or fragment thereof that binds B7-H1 polypeptide and inhibits or reduces binding of the B7-H1 polypeptide to the PD-1 receptor.

19. The composition of claim 18 wherein the fragment comprises the extracellular domain of B7-DC or fragment thereof that binds B7-H1 or the extracellular domain of B7-H1 or fragment thereof that binds B7-DC.

20. The composition of claim 17 wherein the PD-1 antagonist comprises a fusion protein.

21. The composition of claim 20 wherein the fusion proteins binds the PD-1 receptor without triggering signal transduction through the PD-1 receptor.

22. The composition of claim 17 wherein the PD-1 receptor antagonist comprises a B7-H1 polypeptide that binds to B7-DC polypeptide and inhibits or reduces binding of the B7-DC polypeptide to PD-1 receptors.

23. The composition of claim 22 wherein the PD-1 receptor antagonist comprises a fusion protein.

24. A composition comprising an effective amount of a polypeptide to bind PD-1 in vivo without triggering signal transduction through PD-1.

25. The composition of claim 24 wherein the polypeptide comprises a B7-DC or B7-H1 polypeptide modified so that it binds to PD-1 without triggering signal transduction.

26. The composition of claim 24 wherein the polypeptide comprises a variant extracellular domain of B7-DC or B7-H1 modified so that the polypeptide binds to PD-1 without triggering signal transduction through PD-1.

27. A fusion polypeptide comprising:
a) a first fusion partner, and
b) a second fusion partner,
wherein the first fusion partner comprises a variant extracellular domain or fragment thereof of a ligand of PD-1 modified to bind PD-1 without triggering signal transduction through PD-1 and wherein the first fusion partner is fused directly to the second fusion partner, or optionally, is fused to a linker sequence that is fused to the second fusion partner.

28. The fusion polypeptide of claim 27 wherein the second fusion partner comprises one or more domains of an Ig heavy chain constant region.

29. The fusion polypeptide of claim 28 wherein the second polypeptide comprises an amino acid sequence corresponding to the hinge, C_{H}2 and C_{H}3 regions of a human immunoglobulin Cγ1 chain.

30. The fusion polypeptide of claim 27, wherein the first polypeptide comprises the extracellular domain of B7-DC or B7-H1 modified to bind PD-1 without triggering signal transduction through PD-1.
